(19) United States
(12) Patent Application Publication
(10) Pub. No.: US 2022/0025037 A1
(43) Pub. Date: Jan. 27, 2022

(54) ANTIBODY VARIABLE DOMAINS TARGETING DLL3, AND USE THEREOF

(71) Applicant: Dragonfly Therapeutics, Inc., Waltham, MA (US)

(72) Inventors: Hemanta Baruah, Euless, TX (US); Ann F. Cheung, Lincoln, MA (US); Daniel Fallon, Winchester, MA (US); Asya Grinberg, Lexington, MA (US); William Haney, Wayland, MA (US); Steven O'Neil, Wayland, MA (US)

(21) Appl. No.: 17/045,015

(22) PCT Filed: Apr. 3, 2019
(86) PCT No.: PCT/US19/25566

§ 371 Date: Oct. 2, 2020

Related U.S. Application Data
(60) Provisional application No. 62/651,945, filed on Apr. 3, 2018.

(51) Int. Cl. C07K 16/28; C12N 15/86

(52) U.S. Cl. CPC C07K 16/28; C12N 15/86; C07K 2317/24; C07K 2317/31; C07K 2317/77; C07K 2317/92; C07K 2317/94; C07K 2317/33

(57) ABSTRACT

Disclosed are proteins with antibody heavy chain and light chain variable domains that can be paired to form an antigen-binding site targeting DLL3 (Delta-like 3) on a cell, pharmaceutical compositions comprising such proteins, and therapeutic methods using such proteins and pharmaceutical compositions, including for the treatment of cancer.

### CROSS-REFERENCE TO RELATED APPLICATIONS

This application claims the benefit of and priority to U.S. Provisional Patent Application No. 62/651,945, filed Apr. 3, 2018, the disclosure of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Apr. 2, 2019, is named DFY_052WO_SL_ST25.txt and is 61,328 bytes in size.

### FIELD OF THE INVENTION

The invention provides proteins with antibody heavy chain and light chain variable domains that can be paired to form an antigen-binding site targeting Delta like canonical Notch ligand 3 (DLL3) on a cell, pharmaceutical compositions comprising such proteins, and therapeutic methods using such proteins and pharmaceutical compositions, including for the treatment of cancer.

### BACKGROUND

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects. Other types of cancers also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens and to certain immune cells have been described in the literature. See, e.g., WO 2016/134371 and WO 2015/095412.

DLL3 belongs to the delta protein ligand family, and acts as a ligand in the notch signaling pathway. DLL3 is characterized by a DSL domain, EGF repeats and a transmembrane domain. DLL3 has been associated with a variety of neuroendocrine cancers. It is expressed on the surface of tumor cells in about 85% of patients with small-cell lung cancer and large-cell neuroendocrine cancer, but not in healthy tissues. It is also implicated in glioblastoma, Ewing's Sarcoma and other cancers with neuroendocrine phenotype. DLL3 binds to Notch receptors and promotes the proliferation and inhibits the apoptosis of cancer cells.

### SUMMARY OF THE INVENTION

In one aspect, the invention provides an antigen-binding site that binds one or more epitopes on the extracellular domain (ECD) of human DLL3, including one or more of the N-terminus, DSL, EGF1, EGF2, EGF3, EGF4, EGF5, and EGF6 domain of the ECD of DLL3. In some embodiments, an antigen-binding site described herein binds to human DLL3 ECD with high affinity, and with low or little cross-reactivity to human DLL1 and DLL4.

In certain embodiments, the DLL3 antigen-binding site includes an antibody heavy chain variable domain of amino acid sequence at least 90% identical (e.g., 90%, 91%, 92%, 93%, 94%, 95%, 96%, 97%, 98%, 99%, and 100%) to the amino acid sequence of SEQ ID NO:33. In some embodiments, the heavy chain variable domain incorporates amino acid sequences of SEQ ID NO:40 as the first complementarity-determining region 1 (“CDR1”), SEQ ID NO:41 as the second CDR (“CDR2”), and SEQ ID NO:50 as the third CDR (“CDR3”) of SEQ ID NO:33. In some embodiments, the heavy chain variable domain incorporates amino acid sequences of SEQ ID NO:40 as CDR1, SEQ ID NO:41 as CDR2, and SEQ ID NO:47 as CDR3 of SEQ ID NO:33. In some embodiments, the heavy chain variable domain incorporates amino acid sequences of SEQ ID NO:40 as CDR1, SEQ ID NO:41 as CDR2, and SEQ ID NO:46 as CDR3 of SEQ ID NO:33. In some embodiments, the heavy chain variable domain incorporates amino acid sequences of SEQ ID NO:40 as CDR1, SEQ ID NO:41 as CDR2, and SEQ ID NO:49 as CDR3 of SEQ ID NO:33. In some embodiments, the heavy chain variable domain incorporates amino acid sequences of SEQ ID NO:40 as CDR1, SEQ ID NO:41 as CDR2, and SEQ ID NO:48 as CDR3 of SEQ ID NO:33. In some embodiments, the heavy chain variable domain incorporates amino acid sequences of SEQ ID NO:40 as CDR1, SEQ ID NO:41 as CDR2, and SEQ ID NO:42 as CDR3 of SEQ ID NO:33. In some embodiments, the antibody heavy chain variable domain that includes an amino acid sequence at least 90% identical to the amino acid sequence of SEQ ID NO:33 is combined with a light chain variable domain to form an antigen-binding site capable of binding to DLL3. For example, an antibody heavy chain variable domain at least 90% identical to the amino acid sequence of SEQ ID NO:33 can be paired with an antibody light chain variable domain at least 90% identical to the amino acid sequence of SEQ ID NO:34. In certain embodiments, an antibody heavy chain variable domain at least 90% identical to the amino acid sequence of SEQ ID NO:33 can be paired with an antibody light chain variable domain at least 90% identical to the amino acid sequence of SEQ ID NO:34, which incorporates amino acid sequences of SEQ ID NO:43 as CDR1, SEQ ID NO:44 as CDR2, and SEQ ID NO:45 as CDR3 of SEQ ID NO:34.

In certain embodiments, the DLL3 antigen-binding site includes an antibody heavy chain variable domain of amino acid sequence at least 90% identical (e.g., 90%, 91%, 92%, 93%, 94%, 95%, 96%, 97%, 98%, 99%, and 100%) to the amino acid sequence of SEQ ID NO:1. In some embodiments, the heavy chain variable domain incorporates amino acid sequences of SEQ ID NO:9 as CDR1, SEQ ID NO:10 as CDR2, and SEQ ID NO:11 as CDR3 of SEQ ID NO:1. In some embodiments, the antibody heavy chain variable domain that includes an amino acid sequence at least 90% identical to the amino acid sequence of SEQ ID NO:1 is combined with a light chain variable domain to form an antigen-binding site capable of binding to DLL3. For example, an antibody heavy chain variable domain at least 90% identical to the amino acid sequence of SEQ ID NO:1 can be paired with an antibody light chain variable domain at least 90% identical to the amino acid sequence of SEQ ID NO:2. In certain embodiments, an antibody heavy chain variable domain at least 90% identical to the amino acid sequence of SEQ ID NO:1 can be paired with an antibody light chain variable domain at least 90% identical to the amino acid sequence of SEQ ID NO:2, which incorporates amino acid sequences of SEQ ID NO:12 as CDR1, SEQ ID NO:13 as CDR2, and SEQ ID NO:14 as CDR3 of SEQ ID NO:2.

In certain embodiments, the DLL3 antigen-binding site includes an antibody heavy chain variable domain of amino acid sequence at least 90% (e.g., 90%, 91%, 92%, 93%, 94%, 95%, 96%, 97%, 98%, 99%, and 100%) identical to the amino acid sequence SEQ ID NO:3. In some embodiments, the heavy chain variable domain incorporates amino acid sequences of SEQ ID NO:15 as CDR1, SEQ ID NO:16 as CDR2, and SEQ ID NO:17 as CDR3 of SEQ ID NO:3. In some embodiments, the antibody heavy chain variable domain that includes an amino acid sequence at least 90% identical to the amino acid sequence of SEQ ID NO:3 is combined with a light chain variable domain to form an antigen-binding site capable of binding to DLL3. For example, an antibody heavy chain variable domain at least 90% identical to the amino acid sequence of SEQ ID NO:3 can be paired with an antibody light chain variable domain at least 90% identical to the amino acid sequence of SEQ ID NO:4. In certain embodiments, an antibody heavy chain variable domain at least 90% identical to the amino acid sequence of SEQ ID NO:3 can be paired with an antibody light chain variable domain at least 90% identical to the amino acid sequence of SEQ ID NO:4, which incorporates amino acid sequences of SEQ ID NO:18 as CDR1, SEQ ID NO:19 as CDR2, and SEQ ID NO:20 as CDR3 of SEQ ID NO:4.

In certain embodiments, the DLL3 antigen-binding site includes an antibody heavy chain variable domain of amino acid sequence at least 90% (e.g., 90%, 91%, 92%, 93%, 94%, 95%, 96%, 97%, 98%, 99%, and 100%) identical to the amino acid sequence of SEQ ID NO:5. In some embodiments, the heavy chain variable domain incorporates amino acid sequences of SEQ ID NO:21 as CDR1, SEQ ID NO:22 as CDR2, and SEQ ID NO:23 as CDR3 of SEQ ID NO:5. In some embodiments, the antibody heavy chain variable domain that includes an amino acid sequence at least 90% identical to the amino acid sequence of SEQ ID NO:5 is combined with a light chain variable domain to form an antigen-binding site capable of binding to DLL3. For example, an antibody heavy chain variable domain at least 90% identical to the amino acid sequence of SEQ ID NO:5 can be paired with an antibody light chain variable domain at least 90% identical to the amino acid sequence of SEQ ID NO:6. In certain embodiments, an antibody heavy chain variable domain at least 90% identical to the amino acid sequence of SEQ ID NO:5 can be paired with an antibody light chain variable domain at least 90% identical to the amino acid sequence of SEQ ID NO:6, which incorporates amino acid sequences of SEQ ID NO:24 as CDR1, SEQ ID NO:25 as CDR2, and SEQ ID NO:26 as CDR3 of SEQ ID NO:6.

In certain embodiments, the DLL3 antigen-binding site includes an antibody heavy chain variable domain of amino acid sequence at least 90% (e.g., 90%, 91%, 92%, 93%, 94%, 95%, 96%, 97%, 98%, 99%, and 100%) identical to the amino acid sequence of SEQ ID NO:7. In some embodiments, the heavy chain variable domain incorporates amino acid sequences of SEQ ID NO:27 as CDR1, SEQ ID NO:28 as CDR2, and SEQ ID NO:29 as CDR3 of SEQ ID NO:7. In some embodiments, the antibody heavy chain variable domain that includes an amino acid sequence at least 90% identical to the amino acid sequence of SEQ ID NO:7 is combined with a light chain variable domain to form an antigen-binding site capable of binding to DLL3. For example, an antibody heavy chain variable domain at least 90% identical to the amino acid sequence of SEQ ID NO:7 can be paired with an antibody light chain variable domain at least 90% identical to the amino acid sequence of SEQ ID NO:8. In certain embodiments, an antibody heavy chain variable domain at least 90% identical to the amino acid sequence of SEQ ID NO:7 can be paired with an antibody light chain variable domain at least 90% identical to the amino acid sequence of SEQ ID NO:8, which incorporates amino acid sequences SEQ ID NO:30 as CDR1, SEQ ID NO:31 as CDR2, and SEQ ID NO:32 as CDR3 of SEQ ID NO:8.

An antibody heavy chain variable domain of SEQ ID NO: 33, 35, 36, 37, 38, 39, 1, 3, 5, or 7 can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to an antibody constant region, such as a human antibody constant region, a human IgG1 constant region, a human IgG2 constant region, a human IgG3 constant region, or a human IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to a human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, the DLL3 antigen-binding site includes an immunoglobulin heavy chain variable region which comprises the amino acid sequence of SEQ ID NO:33, and the immunoglobulin light chain variable region which comprises the amino acid sequence of SEQ ID NO:34.

In certain embodiments, the DLL3 antigen-binding site includes an immunoglobulin heavy chain variable region which comprises the amino acid sequence of SEQ ID NO:1, and the immunoglobulin light chain variable region which comprises the amino acid sequence of SEQ ID NO:2.

In certain embodiments, the DLL3 antigen-binding site includes an immunoglobulin heavy chain variable region which comprises the amino acid sequence of SEQ ID NO:3, and the immunoglobulin light chain variable region which comprises the amino acid sequence of SEQ ID NO:4.

In certain embodiments, the DLL3 antigen-binding site includes an immunoglobulin heavy chain variable region which comprises the amino acid sequence of SEQ ID NO:5, and the immunoglobulin light chain variable region which comprises the amino acid sequence of SEQ ID NO:6.

In certain embodiments, the DLL3 antigen-binding site includes an immunoglobulin heavy chain variable region which comprises the amino acid sequence of SEQ ID NO:7, and the immunoglobulin light chain variable region which comprises the amino acid sequence of SEQ ID NO:8.

In certain embodiments, the present invention provides a protein that includes one of the DLL3 antigen-binding sites as described above and a second antigen-binding site same or different from the DLL3 antigen-binding site. In certain embodiments, the protein is an antibody.

In certain embodiments, the protein binds to DLL3-expressing cells, which can include but are not limited to small cell lung cancer, large cell neuroendocrine carcinoma, glioblastoma, Ewing's sarcoma, and cancers with neuroendocrine phenotype.

In certain embodiments, the protein has a K_{D} of 10 nM or lower for binding to the ECD of human DLL3, as measured by surface plasmon resonance (SPR).

In another aspect, the invention provides one or more isolated nucleic acids that includes a sequence encoding any of the DLL3 immunoglobulin heavy chain variable domains and light chain variable domains described herein. The invention provides one or more expression vectors that express any of the DLL3 immunoglobulin heavy chain variable domains and light chain variable domains described herein. Similarly the invention provides host cells comprising one or more of the foregoing expression vectors and/or isolated nucleic acids.

Formulations including any of the proteins that include a DLL3-binding domain described herein and methods of enhancing tumor cell death using these proteins and/or formulations are also provided.

In another aspect, the invention provides a method of treating a cancer, for example, a DLL3-associated cancer, in a subject. The method comprises administering to the subject an effective amount of a protein containing any DLL3-binding domain described herein. Exemplary cancers include, for example, small cell lung cancer, large cell neuroendocrine carcinoma, glioblastoma, Ewing's sarcoma, and cancers with neuroendocrine phenotype.

In another aspect, the invention provides a method of inhibiting cancer growth, for example, the growth of a DLL3-associated cancer, in a subject. The method comprises exposing the subject to an effective amount of an antibody comprising any DLL3-binding domain described herein. Exemplary cancers include, for example, small cell lung cancer, large cell neuroendocrine carcinoma, glioblastoma, Ewing's sarcoma, and cancers with neuroendocrine phenotype.

In another aspect, the invention provides an isolated nucleic acid encoding a chimeric antigen receptor (CAR), wherein the nucleic acid comprises a nucleic acid sequence that encodes a DLL3-binding scFv comprising a sequence at least 90%, 95%, or 99% identical to an amino acid sequence selected from SEQ ID NO:51, SEQ ID NO:52, SEQ ID NO:55, SEQ ID NO:56, SEQ ID NO:59, SEQ ID NO:60, SEQ ID NO:63, SEQ ID NO:64, SEQ ID NO:67, and SEQ ID NO:68; a nucleic acid sequence encoding a transmembrane domain; and a nucleic acid sequence encoding an intracellular signaling domain.

In one aspect, the invention provides a chimeric antigen receptor (CAR), wherein the CAR comprises a DLL3-binding scFv comprising amino acid sequence at least 90%, 95%, or 99% identical to an amino acid sequence selected from SEQ ID NO:51, SEQ ID NO:52, SEQ ID NO:55, SEQ ID NO:56, SEQ ID NO:59, SEQ ID NO:60, SEQ ID NO:63, SEQ ID NO:64, SEQ ID NO:67, and SEQ ID NO:68; a transmembrane domain; and an intracellular signaling domain.

In another aspect, the invention provides an immune effector cell comprising the nucleic acid encoding a DLL3-binding scFv comprising a sequence at least 90%, 95%, or 99% identical to an amino acid sequence selected from SEQ ID NO:51, SEQ ID NO:52, SEQ ID NO:55, SEQ ID NO:56, SEQ ID NO:59, SEQ ID NO:60, SEQ ID NO:63, SEQ ID NO:64, SEQ ID NO:67, and SEQ ID NO:68; a nucleic acid sequence encoding a transmembrane domain; and a nucleic acid sequence encoding an intracellular signaling domain.

In another aspect, the invention provides a DLL3/CD3-directed bispecific T-cell engager comprising a protein comprising a sequence at least 90%, 95%, or 99% identical to an amino acid sequence selected from SEQ ID NO:51, SEQ ID NO:52, SEQ ID NO:55, SEQ ID NO:56, SEQ ID NO:59, SEQ ID NO:60, SEQ ID NO:63, SEQ ID NO:64, SEQ ID NO:67, and SEQ ID NO:68.

In another aspect, the invention provides an antibody-drug conjugate comprising a protein comprising a sequence at least 90%, 95%, or 99% identical to an amino acid sequence selected from SEQ ID NO:51, SEQ ID NO:52, SEQ ID NO:55, SEQ ID NO:56, SEQ ID NO:59, SEQ ID NO:60, SEQ ID NO:63, SEQ ID NO:64, SEQ ID NO:67, and SEQ ID NO:68.

In another aspect, the invention provides an immunocytokine comprising a sequence at least 90%, 95%, or 99% identical to an amino acid sequence selected from SEQ ID NO:51, SEQ ID NO:52, SEQ ID NO:55, SEQ ID NO:56, SEQ ID NO:59, SEQ ID NO:60, SEQ ID NO:63, SEQ ID NO:64, SEQ ID NO:67, and SEQ ID NO:68, connected to a cytokine.

The present invention further provides a method of enhancing cancer cell death, the method comprising exposing a DLL3-expressing tumor to an immune effector cell, a DLL3/CD3-directed bispecific T-cell engager, an antibody-drug conjugate, or an immunocytokine of the present invention.

The present invention further provides a method of treating a DLL3-expressing cancer, the method comprising administering to a subject in need thereof an immune effector cell, a DLL3/CD3-directed bispecific T-cell engager, an antibody-drug conjugate, or an immunocytokine of the present invention.

These and other aspects and advantages of the invention are illustrated by the following figures, detailed description and claims.

### DESCRIPTION OF THE DRAWINGS

The invention can be more completely understood with reference to the following drawings.

FIG. 1 shows binding kinetics of murine anti-DLL3 antibodies to the ECD of DLL3 obtained by SPR analysis at 37° C. Antibodies demonstrate range of affinities from <0.011 up to 8.44 nM. Stemcentrx antibody was used as a control.

FIG. 2A are line graphs showing binding kinetics of a murine anti-DLL3 antibody that includes the 5E7 clone to the different domains of DLL3 obtained by SPR analysis at 37° C. FIG. 2B is an illustration of different domains of DLL3, including N-terminus (N-term), DSL domain, EGF domains, and C-terminus (C). “PM” indicates plasma membrane.

FIG. 3A is a time-response curve showing the epitope binning of an anti-DLL3 antibody that includes the 9E6 clone with the Stemcentrx anti-DLL3 antibody measured by SPR analysis at 25° C. FIG. 3B is a time-response curve showing the epitope binning of an anti-DLL3 antibody that includes the 2F7 clone with the Stemcentrx anti-DLL3 antibody measured by SPR analysis at 25° C. FIG. 3C is a time-response curve showing the epitope binning of an anti-DLL3 antibody that includes the 5E7 clone with the Stemcentrx anti-DLL3 antibody measured by SPR analysis at 25° C. Anti-DLL3 antibodies were captured in a uniform orientation on the anti-mouse Fc Biacore chip, followed by injection of DLL3 ECD, followed by injection of the Stemcentrx antibody.

FIG. 4 is a bar graph showing melting temperatures of different anti-DLL3 antibodies by differential scanning fluorimetry. All antibodies demonstrate melting temperatures above 70° C.

FIG. 5 are line graphs showing binding of the anti-DLL3 antibodies to human DLL3 in a dose-dependent manner. Antibody for DLL3 from R&D System (MAB4215) was used as the positive control.

FIGS. 6A-6B are line graphs showing limited cross-reactive binding of anti-DLL3 antibodies to recombinant DLL1 and DLL4. FIG. 6A shows binding of the anti-DLL3 antibodies to human DLL1, and DLL1 antibody (BioLegend—MHD1-314) was used as the positive control. FIG. 6B shows binding of anti-DLL3 antibodies to human DLL4, and DLL4 antibody (Biolegend—MHD4-46) was used as the positive control.

FIG. 7A is a flow cytometry histogram profile showing the binding of the anti-DLL3 antibodies (2 μg/mL) to DLL3 expressed on NCI-H82 cells. FIG. 7B are line graphs showing a dose-response binding profile of the anti-DLL3 antibodies to DLL3 on NCI-H82 cells.

FIG. 8A are line graphs showing the extent of antibody internalization on SHP-77 cells expressing DLL3 after 1-3 hours of incubation of the antibodies with the cells. FIG. 8B are line graphs showing the extent of antibody internalization on DMS-79 cells expressing DLL3 after 1-3 hours of incubation of the antibodies with the cells.

FIG. 9 is a representation of a protein that contains a DLL3-binding site (left arm), a second antigen-binding site (right arm), and an Fc domain.

FIG. 10 is a representation of a protein that includes a DLL3-binding site and a second antigen-binding site, either one of which can be in a scFv format, and an Fc domain.

FIG. 11 is a representation of a DLL3-binding protein in the Triomab form, which is a trifunctional, bispecific antibody that maintains an IgG-like shape. This chimera consists of two half antibodies, each with one light and one heavy chain, that originate from two parental antibodies. Triomab form may be a heterodimeric construct containing ½ of rat antibody and ½ of mouse antibody.

FIG. 12 is a representation of a DLL3-binding protein in the KiH Common Light Chain (LC) form, which involves the knobs-into-holes (KIHs) technology. KiH is a heterodimer containing 2 Fabs binding to target 1 and 2, and an Fc stabilized by heterodimerization mutations. TriNKET in the KiH format may be a heterodimeric construct with 2 fabs binding to target 1 and target 2, containing two different heavy chains and a common light chain that pairs with both heavy chains.

FIG. 13 is a representation of a DLL3-binding protein in the dual-variable domain immunoglobulin (DVD-Ig™) form, which combines the target binding domains of two monoclonal antibodies via flexible naturally occurring linkers, and yields a tetravalent IgG-like molecule. DVD-Ig™ is a homodimeric construct where variable domain targeting antigen 2 is fused to the N-terminus of variable domain of Fab targeting antigen 1 Construct contains normal Fc.

FIG. 14 is a representation of a DLL3-binding protein in the Orthogonal Fab interface (Ortho-Fab) form, which is a heterodimeric construct that contains 2 Fabs binding to target1 and target 2 fused to Fc. LC-HC pairing is ensured by orthogonal interface. Heterodimerization is ensured by mutations in the Fc.

FIG. 15 is a representation of a DLL3-binding protein in the 2-in-1 Ig format.

FIG. 16 is a representation of a DLL3-binding protein in the ES form, which is a heterodimeric construct containing two different Fabs binding to target 1 and target 2 fused to the Fc. Heterodimerization is ensured by electrostatic steering mutations in the Fc.

FIG. 17 is a representation of a DLL3-binding protein in the Fab Arm Exchange form: antibodies that exchange Fab arms by swapping a heavy chain and attached light chain (half-molecule) with a heavy-light chain pair from another molecule, resulting in bispecific antibodies. Fab Arm Exchange form (cFae) is a heterodimer containing 2 Fabs binding to target 1 and 2, and an Fc stabilized by heterodimerization mutations.

FIG. 18 is a representation of a DLL3-binding protein in the SEED Body form, which is a heterodimer containing 2 Fabs binding to target 1 and 2, and an Fc stabilized by heterodimerization mutations.

FIG. 19 is a representation of a DLL3-binding protein in the LuZ-Y form, in which a leucine zipper is used to induce heterodimerization of two different HCs. The LuZ-Y form is a heterodimer containing two different scFabs binding to target 1 and 2, fused to Fc. Heterodimerization is ensured through leucine zipper motifs fused to C-terminus of Fc.

FIG. 20 is a representation of a DLL3-binding protein in the Cov-X-Body form.

FIGS. 21A-21B are representations of a DLL3-binding protein in the κλ-Body forms, which are heterodimeric constructs with two different Fabs fused to Fc stabilized by heterodimerization mutations: FabI targeting antigen 1 contains kappa LC, while second Fab targeting antigen 2 contains lambda LC. FIG. 21A is an exemplary representation of one form of a κλ-Body; FIG. 21B is an exemplary representation of another κλ-Body.

FIG. 22 is a representation of a DLL3-binding protein in an Oasc-Fab heterodimeric construct that includes Fab binding to target 1 and scFab binding to target 2 fused to Fc. Heterodimerization is ensured by mutations in the Fc.

FIG. 23 is a representation of a DLL3-binding protein in a DuetMab, which is a heterodimeric construct containing two different Fabs binding to antigens 1 and 2, and Fc stabilized by heterodimerization mutations. Fab 1 and 2 contain differential S-S bridges that ensure correct light chain (LC) and heavy chain (HC) pairing.

FIG. 24 is a representation of a DLL3-binding protein in a CrossmAb, which is a heterodimeric construct with two different Fabs binding to targets 1 and 2 fused to Fc stabilized by heterodimerization. CL and CH1 domains and VH and VL domains are switched, e.g., CH1 is fused in-line with VL, while CL is fused in-line with VH.

FIG. 25 is a representation of a DLL3-binding protein in a Fit-Ig, which is a homodimeric construct where Fab binding to antigen 2 is fused to the N-terminus of HC of Fab that binds to antigen 1. The construct contains wild-type Fc.

FIG. 26 is a series of sensograms generated from a Biacore analysis of DLL3-His binding to murine and humanized variants of the 5E7 antibody.

FIG. 27 is a graph showing the binding of humanized variants of the 5E7 antibody to RPMI-8226 cells transduced to express DLL3, compared to binding of a chimeric protein of murine 5E7 variable regions and human IgG1/Igx constant regions to the same cells.

### DETAILED DESCRIPTION

The invention provides an antigen-binding site that binds one or more epitopes on the extracellular domain (ECD) of human DLL3, including one or more of the N-terminus, DSL, EGF1, EGF2, EGF3, EGF4, EGF5, and EGF6 of the ECD of DLL3. In some embodiments, the antigen-binding site binds to human DLL3 ECD with high affinity, and with little cross-reactivity to human DLL1 and DLL4.

The invention provides proteins that include the DLL3-binding site described herein and can bind DLL3 on a cancer cell. The invention provides pharmaceutical compositions comprising such proteins, and therapeutic methods using such proteins and pharmaceutical compositions for the treatment of cancer. Various aspects of the invention are set forth in the sections below; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide. All the amino acid positions in heavy or light chain variable regions disclosed herein are numbered according to Kabat numbering.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Various features and aspects of the invention are discussed in more detail below.

### I. DLL3-Binding Sites

In certain embodiments, the present invention provides a DLL3 antigen-binding site that includes an antibody heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:39, and an antibody light chain variable domain that includes an amino acid sequence at least 90% (e.g., 91%, 92%, 93%, 94%, 95%, 96%, 97%, 98%, 99%, or 100%) identical to SEQ ID NO:34. In certain embodiments, the DLL3 antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:39, includes a CDR1 sequence represented by the amino acid sequence of SEQ ID NO:40, a CDR2 sequence represented by the amino acid sequence of SEQ ID NO:41, and a CDR3 sequence represented by the amino acid sequence of SEQ ID NO:50. In certain embodiments, the DLL3 antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:34, includes a CDR1 sequence represented by the amino acid sequence of SEQ ID NO:43, a CDR2 sequence represented by the amino acid sequence of SEQ ID NO:44, and a CDR3 sequence represented by the amino acid sequence of SEQ ID NO:45. In certain embodiments, the antibody heavy chain variable domain and the antibody light chain variable domain are humanized (e.g., derived from human germline VH1-3 and human germline VK1-39, respectively). In certain embodiments, the antibody heavy chain variable domain is derived from human germline VH1-3 and comprises mutations at positions 44, 71, and 76 relative to human germline VH1-3. In certain embodiments, the antibody heavy chain variable domain is derived from human germline VH1-3 and comprises amino acids G, A, and N at positions 44, 71, and 76, respectively. In certain embodiments, the antibody light chain variable domain is derived from human germline VK1-39 and comprises mutations at positions 2, 36, and 42 relative to human germline VK1-39. In certain embodiments, the antibody heavy chain variable domain is derived from human germline VK1-39 and comprises amino acids V, L, and Q at positions 2, 36, and 42, respectively.

In certain embodiments, the present invention provides a DLL3 antigen-binding site that includes an antibody heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:33, and an antibody light chain variable domain that includes an amino acid sequence at least 90% (e.g., 91%, 92%, 93%, 94%, 95%, 96%, 97%, 98%, 99%, or 100%) identical to SEQ ID NO:34. In certain embodiments, the DLL3 antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:33, includes a CDR1 sequence represented by the amino acid sequence of SEQ ID NO:40, a CDR2 sequence represented by the amino acid sequence of SEQ ID NO:41, and a CDR3 sequence represented by the amino acid sequence of SEQ ID NO:42. In certain embodiments, the DLL3 antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:34, includes a CDR1 sequence represented by the amino acid sequence of SEQ ID NO:43, a CDR2 sequence represented by the amino acid sequence of SEQ ID NO:44, and a CDR3 sequence represented by the amino acid sequence of SEQ ID NO:45. In certain embodiments, the antibody heavy chain variable domain and the antibody light chain variable domain are humanized (e.g., derived from human germline VH1-3 and human germline VK1-39, respectively). In certain embodiments, the antibody heavy chain variable domain is derived from human germline VH1-3 and comprises mutations at positions 44, 71, and 76 relative to human germline VH1-3. In certain embodiments, the antibody heavy chain variable domain is derived from human germline VH1-3 and comprises amino acids G, A, and N at positions 44, 71, and 76, respectively. In certain embodiments, the antibody light chain variable domain is derived from human germline VK1-39 and comprises mutations at positions 2, 36, and 42 relative to human germline VK1-39. In certain embodiments, the antibody heavy chain variable domain is derived from human germline VK1-39 and comprises amino acids V, L, and Q at positions 2, 36, and 42, respectively.

In certain embodiments, the present invention provides a DLL3 antigen-binding site that includes an antibody heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:35, and an antibody light chain variable domain that includes an amino acid sequence at least 90% (e.g., 91%, 92%, 93%, 94%, 95%, 96%, 97%, 98%, 99%, or 100%) identical to SEQ ID NO:34. In certain embodiments, the DLL3 antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:35, includes a CDR1 sequence represented by the amino acid sequence of SEQ ID NO:40, a CDR2 sequence represented by the amino acid sequence of SEQ ID NO:41, and a CDR3 sequence represented by the amino acid sequence of SEQ ID NO:46. In certain embodiments, the DLL3 antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:34, includes a CDR1 sequence represented by the amino acid sequence of SEQ ID NO:43, a CDR2 sequence represented by the amino acid sequence of SEQ ID NO:44, and a CDR3 sequence represented by the amino acid sequence of SEQ ID NO:45. In certain embodiments, the antibody heavy chain variable domain and the antibody light chain variable domain are humanized (e.g., derived from human germline VH1-3 and human germline VK1-39, respectively). In certain embodiments, the antibody heavy chain variable domain is derived from human germline VH1-3 and comprises mutations at positions 44, 71, and 76 relative to human germline VH1-3. In certain embodiments, the antibody heavy chain variable domain is derived from human germline VH1-3 and comprises amino acids G, A, and N at positions 44, 71, and 76, respectively. In certain embodiments, the antibody light chain variable domain is derived from human germline VK1-39 and comprises mutations at positions 2, 36, and 42 relative to human germline VK1-39. In certain embodiments, the antibody heavy chain variable domain is derived from human germline VK1-39 and comprises amino acids V, L, and Q at positions 2, 36, and 42, respectively.

In certain embodiments, the present invention provides a DLL3 antigen-binding site that includes an antibody heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:36, and an antibody light chain variable domain that includes an amino acid sequence at least 90% (e.g., 91%, 92%, 93%, 94%, 95%, 96%, 97%, 98%, 99%, or 100%) identical to SEQ ID NO:34. In certain embodiments, the DLL3 antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:36, includes a CDR1 sequence represented by the amino acid sequence of SEQ ID NO:40, a CDR2 sequence represented by the amino acid sequence of SEQ ID NO:41, and a CDR3 sequence represented by the amino acid sequence of SEQ ID NO:47. In certain embodiments, the DLL3 antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:34, includes a CDR1 sequence represented by the amino acid sequence of SEQ ID NO:43, a CDR2 sequence represented by the amino acid sequence of SEQ ID NO:44, and a CDR3 sequence represented by the amino acid sequence of SEQ ID NO:45. In certain embodiments, the antibody heavy chain variable domain and the antibody light chain variable domain are humanized (e.g., derived from human germline VH1-3 and human germline VK1-39, respectively). In certain embodiments, the antibody heavy chain variable domain is derived from human germline VH1-3 and comprises mutations at positions 44, 71, and 76 relative to human germline VH1-3. In certain embodiments, the antibody heavy chain variable domain is derived from human germline VH1-3 and comprises amino acids G, A, and N at positions 44, 71, and 76, respectively. In certain embodiments, the antibody light chain variable domain is derived from human germline VK1-39 and comprises mutations at positions 2, 36, and 42 relative to human germline VK1-39. In certain embodiments, the antibody heavy chain variable domain is derived from human germline VK1-39 and comprises amino acids V, L, and Q at positions 2, 36, and 42, respectively.

In certain embodiments, the present invention provides a DLL3 antigen-binding site that includes an antibody heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:37, and an antibody light chain variable domain that includes an amino acid sequence at least 90% (e.g., 91%, 92%, 93%, 94%, 95%, 96%, 97%, 98%, 99%, or 100%) identical to SEQ ID NO:34. In certain embodiments, the DLL3 antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:37, includes a CDR1 sequence represented by the amino acid sequence of SEQ ID NO:40, a CDR2 sequence represented by the amino acid sequence of SEQ ID NO:41, and a CDR3 sequence represented by the amino acid sequence of SEQ ID NO:48. In certain embodiments, the DLL3 antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:34, includes a CDR1 sequence represented by the amino acid sequence of SEQ ID NO:43, a CDR2 sequence represented by the amino acid sequence of SEQ ID NO:44, and a CDR3 sequence represented by the amino acid sequence of SEQ ID NO:45. In certain embodiments, the antibody heavy chain variable domain and the antibody light chain variable domain are humanized (e.g., derived from human germline VH1-3 and human germline VK1-39, respectively). In certain embodiments, the antibody heavy chain variable domain is derived from human germline VH1-3 and comprises mutations at positions 44, 71, and 76 relative to human germline VH1-3. In certain embodiments, the antibody heavy chain variable domain is derived from human germline VH1-3 and comprises amino acids G, A, and N at positions 44, 71, and 76, respectively. In certain embodiments, the antibody light chain variable domain is derived from human germline VK1-39 and comprises mutations at positions 2, 36, and 42 relative to human germline VK1-39. In certain embodiments, the antibody heavy chain variable domain is derived from human germline VK1-39 and comprises amino acids V, L, and Q at positions 2, 36, and 42, respectively.

In certain embodiments, the present invention provides a DLL3 antigen-binding site that includes an antibody heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:38, and an antibody light chain variable domain that includes an amino acid sequence at least 90% (e.g., 91%, 92%, 93%, 94%, 95%, 96%, 97%, 98%, 99%, or 100%) identical to SEQ ID NO:34. In certain embodiments, the DLL3 antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:38, includes a CDR1 sequence represented by the amino acid sequence of SEQ ID NO:40, a CDR2 sequence represented by the amino acid sequence of SEQ ID NO:41, and a CDR3 sequence represented by the amino acid sequence of SEQ ID NO:49. In certain embodiments, the DLL3 antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:34, includes a CDR1 sequence represented by the amino acid sequence of SEQ ID NO:43, a CDR2 sequence represented by the amino acid sequence of SEQ ID NO:44, and a CDR3 sequence represented by the amino acid sequence of SEQ ID NO:45. In certain embodiments, the antibody heavy chain variable domain and the antibody light chain variable domain are humanized (e.g., derived from human germline VH1-3 and human germline VK1-39, respectively). In certain embodiments, the antibody heavy chain variable domain is derived from human germline VH1-3 and comprises mutations at positions 44, 71, and 76 relative to human germline VH1-3. In certain embodiments, the antibody heavy chain variable domain is derived from human germline VH1-3 and comprises amino acids G, A, and N at positions 44, 71, and 76, respectively. In certain embodiments, the antibody light chain variable domain is derived from human germline VK1-39 and comprises mutations at positions 2, 36, and 42 relative to human germline VK1-39. In certain embodiments, the antibody heavy chain variable domain is derived from human germline VK1-39 and comprises amino acids V, L, and Q at positions 2, 36, and 42, respectively.

In certain embodiments, the present invention provides a DLL3 antigen-binding site that includes an antibody heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:1, and an antibody light chain variable domain that includes an amino acid sequence at least 90% (e.g., 91%, 92%, 93%, 94%, 95%, 96%, 97%, 98%, 99%, or 100%) identical to SEQ ID NO:2. In certain embodiments, the DLL3 antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:1, includes a CDR1 sequence represented by the amino acid sequence of SEQ ID NO:9, a CDR2 sequence represented by the amino acid sequence of SEQ ID NO:10, and a CDR3 sequence represented by the amino acid sequence of SEQ ID NO:11. In certain embodiments, the DLL3 antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:2, includes a CDR1 sequence represented by the amino acid sequence of SEQ ID NO:12, a CDR2 sequence represented by the amino acid sequence of SEQ ID NO:13, and a CDR3 sequence represented by the amino acid sequence of SEQ ID NO:14.

In certain embodiments, the present invention provides a DLL3 antigen-binding site that includes an antibody heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:3, and an antibody light chain variable domain that includes an amino acid sequence at least 90% (e.g., 91%, 92%, 93%, 94%, 95%, 96%, 97%, 98%, 99%, or 100%) identical to SEQ ID NO:4. In certain embodiments, the DLL3 antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:3, includes a CDR1 sequence represented by the amino acid sequence of SEQ ID NO:15, a CDR2 sequence represented by the amino acid sequence of SEQ ID NO:16, and a CDR3 sequence represented by the amino acid sequence of SEQ ID NO:17. In certain embodiments, the DLL3 antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:4, includes a CDR1 sequence represented by the amino acid sequence of SEQ ID NO:18, a CDR2 sequence represented by the amino acid sequence of SEQ ID NO:19, and a CDR3 sequence represented by the amino acid sequence of SEQ ID NO:20.

In certain embodiments, the present invention provides a DLL3 antigen-binding site that includes an antibody heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:5, and an antibody light chain variable domain that includes an amino acid sequence at least 90% (e.g., 91%, 92%, 93%, 94%, 95%, 96%, 97%, 98%, 99%, or 100%) identical to SEQ ID NO:6. In certain embodiments, the DLL3 antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:5, includes a CDR1 sequence represented by the amino acid sequence of SEQ ID NO:21, a CDR2 sequence represented by the amino acid sequence of SEQ ID NO:22, and a CDR3 sequence represented by the amino acid sequence of SEQ ID NO:23. In certain embodiments, the DLL3 antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:6, includes a CDR1 sequence represented by the amino acid sequence of SEQ ID NO:24, a CDR2 sequence represented by the amino acid sequence of SEQ ID NO:25, and a CDR3 sequence represented by the amino acid sequence of SEQ ID NO:26.

In certain embodiments, the present invention provides a DLL3 antigen-binding site that includes an antibody heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:7, and an antibody light chain variable domain that includes an amino acid sequence at least 90% (e.g., 91%, 92%, 93%, 94%, 95%, 96%, 97%, 98%, 99%, or 100%) identical to SEQ ID NO:8. In certain embodiments, the DLL3 antigen-binding site that includes an antibody heavy chain variable domain with an amino acid sequence at least 90% (e.g., 91%, 92%, 93%, 94%, 95%, 96%, 97%, 98%, 99%, or 100%) identical to the amino acid sequence of SEQ ID NO:7, includes a CDR1 sequence represented by the amino acid sequence of SEQ ID NO:27, a CDR2 sequence represented by the amino acid sequence of SEQ ID NO:28, and a CDR3 sequence represented by the amino acid sequence of SEQ ID NO:29. In certain embodiments, the DLL3 antigen-binding site that includes an antibody light chain variable domain with an amino acid sequence at least 90% (e.g., 91%, 92%, 93%, 94%, 95%, 96%, 97%, 98%, 99%, or 100%) identical to the amino acid sequence of SEQ ID NO:8, includes a CDR1 sequence represented by the amino acid sequence of SEQ ID NO:30, a CDR2 sequence represented by the amino acid sequence of SEQ ID NO:31, and a CDR3 sequence represented by the amino acid sequence of SEQ ID NO:32.

In each of the foregoing embodiments, it is contemplated herein that immunoglobulin heavy chain variable region sequences and/or light chain variable region sequences that pair to bind DLL3 may contain amino acid alterations (e.g., at least 1, 2, 3, 4, 5, 6, 7, 8, 9 or 10 amino acid substitutions, deletions, or additions) in the framework regions of the heavy and/or light chain variable regions without affecting their ability to bind to DLL3 significantly.

Table 1 lists peptide sequences of heavy chain variable domains and light chain variable domains that, in combination, can bind to DLL3. The DLL3-binding domains can vary in their binding affinities to DLL3. The CDR sequences provided in Table 1 are determined under Kabat. Table 1 also lists scFv forms of the DLL3-binding heavy and light chain variable domains. The exemplary nucleic acid sequences listed in Table 1 are predicted possible nucleic acid sequences that the listed corresponding peptide sequences originated from, and were generated using EMBL-EBI's Protein Sequence Back-translation program.

TABLE 1

| | VH | VL |
|---|---|---|
| 2F7 | QVQLQQSGAELMKPGASVKLSCKATGYTFTGYWI | NIMMTQSPSSLAVSAGEKVTMSCKSSQSVLYSSNQK |
| | DWIKQRPGHGLEWVGEILPGSDNINYNEKFRGKA | NYLAWYQQKPGQSPRLLIYWASTRASGVPDRFTGSG |
| | TFTADTSSNTAYQILSSLTTEDSAIYFCARCGTG | SGTDFTLTITNIQPEDLAVYYCHQFLSSTWTFGGGT |
| | PWFTYWGQGTLVTVSA [SEQ ID NO: 1] | KLEIK [SEQ ID NO: 2] |
| | CDR1: GYWID [SEQ ID NO: 9] | CDR1: KSSQSVLYSSNQKNYLA |
| | CDR2: EILPGSDNINYNEKFRG | [SEQ ID NO: 12] |
| | [SEQ ID NO: 10] | CDR2: WASTRAS [SEQ ID NO: 13] |
| | CDR3: CGTGPWFTY [SEQ ID NO: 11] | CDR3: HQFLSSTWT [SEQ ID NO: 14] |
| 9E6 or 10F5 | QLQLVQSGPELMRPGETVKISCKASGYTFTTYGM | DIVMTQSPSSLSVSAGEKVTMSCKSSQSLLNSGNQK |
| | NWVKQAPGKGLKWVGWINTYSGVPTYADDFKGRF | NYLAWYQQKPGQPPKLLIYGASTRESGVPDRFTGSG |
| | AFSLESSASTAFLQINNLKDEDTATYFCARFGNY | SGTDFTLTISSVQAEDLAVYYCQNDHIYPYTFGGGT |
| | GFDCWGQGTTLTVSS [SEQ ID NO: 3] | KLEIK [SEQ ID NO: 4] |
| | CDR1: TYGMN [SEQ ID NO: 15] | CDR1 KSSQSLLNSGNQKNYLA |
| | CDR2: WINTYSGVPTYADDFKG | [SEQ ID NO: 18] |
| | [SEQ ID NO: 16] | CDR2: GASTRES [SEQ ID NO: 19] |
| | CDR3: FGNYGFDC [SEQ ID NO: 17] | CDR3: QNDHIYPYT [SEQ ID NO: 20] |
| 5E7 | EVQLQQSGAELVRPGASVKLSCTASGFNIKDDYI | DVLMTQTPLTLSVPIGQPASISCKSSQSLLHSNGKT |
| | HWVKQWPEQGLEWIGWIDSENGDTEYASKFQGKA | YLNWLLQRPGQSPKLLIYLVSKLESGVPDRFSGSGS |
| | TMTADTSSNTAYLQLSGLTSEDTAVYYCTTSSYY | GTDFTLKISRVEAEDLGVYYCLQTTHLYTFGGGTKL |
| | SYDLFVYWGQGTLVTVSA [SEQ ID NO: 5] | EIK [SEQ ID NO: 6] |
| | CDR1: DDYIH [SEQ ID NO: 21] | CDR1:KSSQSLLHSNGKTYLN |
| | CDR2: WIDSENGDTEYASKFQG | [SEQ ID NO: 24] |
| | [SEQ ID NO: 22] | CDR2: LVSKLES [SEQ ID NO: 25] |
| | CDR3: SSYYSYDLFVY [SEQ ID NO: 23] | CDR3: LQTTHLYT [SEQ ID NO: 26] |
| 2H6 | QIQLVQSGPELKKPGETVKISCKASGYTFTTYGV | DIVMTQSPSSLSVSAGEKVTMSCKSSQSLVNSGNQK |
| | NWVKQAPGKGLKWMGWINTYSGVPTYADDFKGRF | NYLAWYQQKPGQPPKLLISGASTRESGVPDRFTGSG |
| | AFSLETIATTAYLQINNLKNEDTATYFCARFGNY | SGTDFTLTISSVQAEDLAVYYCQNDHNYPTYFGGGT |
| | GFDYWGQGTTLTVSS [SEQ ID NO: 7] | KLEIK [SEQ ID NO: 8] |
| | CDR1: TYGVN [SEQ ID NO: 27] | CDR1: KSSQSLVNSGNQKNYLA |
| | CDR2: WINTYSGVPTYADDFKG | [SEQ ID NO: 30] |
| | [SEQ ID NO: 28] | CDRR2: GASTRES [SEQ ID NO: 31] |
| | CDR3: FGNYGFDY [SEQ ID NO: 29] | CDR3: QNDHNYPYT [SEQ ID NO: 32} |
| h5E7 | QVQLVQSGAEVKKPGASVKVSCKASGFNIKDDYI | DVQMTQSPSSLSASVGDRVTITCKSSQSLLHSNGKT |
| | HWVRQAPGQGLEWMGWIDSENGDTEYASKFQGRV | YLNWLQQKPGQAPKLLLYLVSKLESGVPSRFSGSGS |
| | TITADTSANTAYMELSSLRSEDTAVYYCATSSYY | GTDYTLTISSLQPEDFATYYCLQTTHLYTFGQGTKL |
| | SYDLFVYWGQGTLVTVSS [SEQ ID NO: 33] | EIK [SEQ ID NO: 34] |
| | CDR1: DDYIH [SEQ ID NO: 40] | CDR1: KSSQSLLHSNGKTYLN |
| | CDR2: WIDSENGDTEYASKFQG | [SEQ ID NO: 43] |
| | [SEQ ID NO: 41] | CDR2: LVSKLES [SEQ ID NO: 44] |
| | CDR3: SSYYSYDLFVY [SEQ ID NO: 42] | CDR3: LQTTHLYT[SEQ ID NO: 45] |
| h5E7-YD-C6 | QVQLVQSGAEVKKPGASVKVSCKASGFNIKDDYI | DVQMTQSPSSLSASVGDRVTITCKSSQSLLHSNGKT |
| | HWVRQAPGQGLEWMGWIDSENGDTEYASKFQGRV | YLNWLQQKPGQAPKLLLYLVSKLESGVPSRFSGSGS |
| | TITADTSANTAYMELSSLRSEDTAVYYCATSEYY | GTDYTLTISSLQPEDFATYYCLQTTHLYTFGQGTKL |
| | SYDLFVYWGQGTLVTVSS [SEQ ID NO: 35] | EIK [SEQ ID NO: 34] |
| | CDR1: DDYIH [SEQ ID NO: 40] | CDR1: KSSQSLLHSNGKTYLN |
| | CDR2: WIDSENGDTEYASKFQG | [SEQ ID NO: 43] |
| | [SEQ ID NO: 41] | CDR2: LVSKLES [SEQ ID NO: 44] |
| | CDR3: SEYYSYDLFVY [SEQ ID NO: 46] | CDR3: LQTTHLYT [SEQ ID NO: 45] |
| h5E7-YD-F3 | QVQLVQSGAEVKKPGASVKVSCKASGFNIKDDYI | DVQMTQSPSSLSASVGDRVTITCKSSQSLLHSNGKT |
| | HWVRQAPGQGLEWMGWIDSENGDTEYASKFQGRV | YLNWLQQKPGQAPKLLLYLVSKLESGVPSRFSGSGS |
| | TITADTSANTAYMELSSLRSEDTAVYYCATSSYW | GTDYTLTISSLQPEDFATYYCLQTTHLYTFGQGTKL |
| | SYDLLVYWGQGTLVTVSS [SEQ ID NO: 36] | EIK [SEQ ID NO: 34] |
| | CDR1: DDYIH [SEQ ID NO: 40] | CDR1: KSSQSLLHSNGKTYLN |
| | CDR2: WIDSENGDTEYASKFQG | [SEQ ID NO: 43] |
| | [SEQ ID NO: 41] | CDR2: LVSKLES [SEQ ID NO: 44] |
| | CDR3: SSYWSYDLLVY [SEQ ID NO: 47] | CDR3: LQTTHLYT [SEQ ID NO: 45] |
| h5E7-YD-A6 | QVQLVQSGAEVKKPGASVKVSCKASGFNIKDDYI | DVQMTQSPSSLSASVGDRVTITCKSSQSLLHSNGKT |
| | HWVRQAPGQGLEWMGWIDSENGDTEYASKFQGRV | YLNWLQQKPGQAPKLLLYLVSKLESGVPSRFSGSGS |
| | TITADTSANTAYMELSSLRSEDTAVYYCATSSYW | GTDYTLTISSLQPEDFATYYCLQTTHLYTFGQGTKL |
| | SYDLFVYWGQGTLVTVSS [SEQ ID NO: 37] | EIK [SEQ ID NO: 34] |
| | CDR1: DDYIH [SEQ ID NO: 40] | CDR1: KSSQSLLHSNGKTYLN |
| | CDR2: WIDSENGDTEYASKFQG | [SEQ ID NO: 43] |
| | [SEQ ID NO: 41] | CDR2: LVSKLES [SEQ ID NO: 44] |
| | CDR3: SSYWSYDLFVY [SEQ ID NO: 48] | CDR3: LQTTHLYT [SEQ ID NO: 45] |
| h5E7-YD-B5 | QVQLVQSGAEVKKPGASVKVSCKASGFNIKDDYI | DVQMTQSPSSLSASVGDRVTITCKSSQSLLHSNGKT |
| | HWVRQAPGQGLEWMGWIDSENGDTEYASKFQGRV | YLNWLQQKPGQAPKLLLYLVSKLESGVPSRFSGSGS |
| | TITADTSANTAYMELSSLRSEDTAVYYCATSTYW | GTDYTLTISSLQPEDFATYYCLQTTHLYTFGQGTKL |
| | SYDLFVYWGQGTLVTVSS [SEQ ID NO: 38] | EIK [SEQ ID NO: 34] |
| | CDR1: DDYIH [SEQ ID NO: 40] | CDR1: KSSQSLLHSNGKTYLN |
| | CDR2: WIDSENGDTEYASKFQG | [SEQ ID NO: 43] |
| | [SEQ ID NO: 41] | CDR2: LVSKLES [SEQ ID NO: 44] |
| | CDR3: STYWSYDLFVY [SEQ ID NO: 49] | CDR3: LQTTHLYT [SEQ ID NO: 45] |
| h5E7 | QVQLVQSGAEVKKPGASVKVSCKASGFNIKDDYI | DVQMTQSPSSLSASVGDRVTITCKSSQSLLHSNGKT |
| variants | HWVRQAPGQGLEWMGWIDSENGDTEYASKFQGRV | YLNWLQQKPGQAPKLLLYLVSKLESGVPSRFSGSGS |
| consensus | TITADTSANTAYMELSSLRSEDTACYYCATSX₁ | GTDYTLTISSLQPEDFATYYCLQTTHLYTFGQGTKL |
| | YX₂SYDLX₃VYWGQGTLVTVSS, wherein | EIK [SEQ ID NO: 34] |
| | X₁ is S, T, or E; | CDR1: KSSQSLLHSNGKTYLN |
| | X₂ is Y or W; and | [SEQ ID NO: 43] |
| | X₃ is F or L [SEQ ID NO: 39] | CDR2: LVSKLES [SEQ ID NO: 44] |
| | CDR1: DDYIH [SEQ ID NO: 40] | CDR3: LQTTHLYT [SEQ ID NO: 45] |
| | CDR2: WIDSENGDTEYASKFQG | |
| | [SEQ ID NO: 41] | |
| | CDR3: SX₁YX₂SYDLX₃VY, | |
| | wherein | |
| | X₁ is S, T, or E; | |
| | X₂ is Y or W; and | |
| | X₃ is F or L [SEQ ID NO: 50] | |

| | |
|---|---|
| scFv of | QVQLVQSGAEVKKPGASVKVSCKASGFNIKDDYIHWVRQAPGQCLEWMGWIDSENGDTEYASKFQGRVTIT |
| h5E7 | ADTSANTAYMELSSLRSEDTAVYYCATSSYYSYDLFVYWGQGTLVTVSSGGGGSGGGGSGGGGSGGGGSDV |
| | QMTQSPSSLSASVGDRVTITCKSSQSLLHSNGKTYLNWLQQKPGQAPKLLLYLVSKLESGVPSRFSGSGSG |
| | TDYTLTISSLQPEDFATYYCLQTTHLYTFGCGTKLEIK [SEQ ID NO: 51] |
| | DVQMTQSPSSLSASVGDRVTITCKSSQSLLHSNGKTYLNWLQQKPGQAPKLLLYLVSKLESGVPSRFSGSG |
| | SGTDYTLTISSLQPEDFATYYCLQTTHLYTFGCGTKLEIKGGGGSGGGGSGGGGSGGGGSQVQLVQSGAEV |
| | KKPGASVKVSCKASGFNIKDDYIHWVRQAPGQCLEWMGWIDSENGDTEYASKFQGRVTITADTSANTAYME |
| | LSSLRSEDTAVYYCATSSYYSYDLFVYWGQGTLVTVSS [SEQ ID NO: 52] |
| Exemplary | CAGGTGCAGCTGGTGCAGAGCGGCGCCGAGGTGAAGAAGCCCGGCGCCAGCGTGAAGGTGAGCTGCAAGGC |
| nucleotide | CAGCGGCTTCAACATCAAGGACGACTACATCCACTGGGTGAGGCAGGCCCCCGGCCAGTGCCTGGAGTGGA |
| sequence of | TGGGCTGGATCGACAGCGAGAACGGCGACACCGAGTACGCCAGCAAGTTCCAGGGCAGGGTGACCATCACC |
| h5E7 scFv | GCCGACACCAGCGCCAACACCGCCTACATGGAGCTGAGCAGCCTGAGGAGCGAGGACACCGCCGTGTACTA |
| | CTGCGCCACCAGCAGCTACTACAGCTACGACCTGTTCGTGTACTGGGGCCAGGGCACCCTGGTGACCGTGA |
| | GCAGCGGCGGCGGCGGCAGCGGCGGCGGCGGCAGCGGCGGCGGCGGCAGCGGCGGCGGCGGCAGCGACGTG |
| | CAGATGACCCAGAGCCCCAGCAGCCTGAGCGCCAGCGTGGGCGACAGGGTGACCATCACCTGCAAGAGCAG |
| | CCAGAGCCTGCTGCACAGCAACGGCAAGACCTACCTGAACTGGCTGCAGCAGAAGCCCGGCCAGGCCCCCA |
| | AGCTGCTGCTGTACCTGGTGAGCAAGCTGGAGAGCGGCGTGCCCAGCAGGTTCAGCGGCAGCGGCAGCGGC |
| | ACCGACTACACCCTGACCATCAGCAGCCTGCAGCCCGAGGACTTCGCCACCTACTACTGCCTGCAGACCAC |
| | CCACCTGTACACCTTCGGCTGCGGCACCAAGCTGGAGATCAAG [SEQ ID NO: 53] |
| | GACGTGCAGATGACCCAGAGCCCCAGCAGCCTGAGCGCCAGCGTGGGCGACAGGGTGACCATCACCTGCAA |
| | GAGCAGCCAGAGCCTGCTGCACAGCAACGGCAAGACCTACCTGAACTGGCTGCAGCAGAAGCCCGGCCAGG |
| | CCCCCAAGCTGCTGCTGTACCTGGTGAGCAAGCTGGAGAGCGGCGTGCCCAGCAGGTTCAGCGGCAGCGGC |
| | AGCGGCACCGACTACACCCTGACCATCAGCAGCCTGCAGCCCGAGGACTTCGCCACCTACTACTGCCTGCA |
| | GACCACCCACCTGTACACCTTCGGCTGCGGCACCAAGCTGGAGATCAAGGGCGGCGGCGGCAGCGGCGGCG |
| | GCGGCAGCGGCGGCGGCGGCAGCGGCGGCGGCGGCAGCCAGGTGCAGCTGGTGCAGAGCGGCGCCGAGGTG |
| | AAGAAGCCCGGCGCCAGCGTGAAGGTGAGCTGCAAGGCCAGCGGCTTCAACATCAAGGACGACTACATCCA |
| | CTGGGTGAGGCAGGCCCCCGGCCAGTGCCTGGAGTGGATGGGCTGGATCGACAGCGAGAACGGCGACACCG |
| | AGTACGCCAGCAAGTTCCAGGGCAGGGTGACCATCACCGCCGACACCAGCGCCAACACCGCCTACATGGAG |
| | CTGAGCAGCCTGAGGAGCGAGGACACCGCCGTGTACTACTGCGCCACCAGCAGCTACTACAGCTACGACCT |
| | GTTCGTGTACTGGGGCCAGGGCACCCTGGTGACCGTGAGCAGC [SEQ ID NO: 54] |
| scFv of | QVQLVQSGAEVKKPGASVKVSCKASGFNIKDDYIHWVRQAPGQCLEWMGWIDSENGDTEYASKFQGRVTIT |
| h5E7-YD-C6 | ADTSANTAYMELSSLRSEDTAVYYCATSEYYSYDLFVYWGQGTLVTVSSGGGGSGGGGSGGGGSGGGGSDV |
| | QMTQSPSSLSASVGDRVTITCKSSQSLLHSNGKTYLNWLQQKPGQAPKLLLYLVSKLESGVPSRFSGSGSG |
| | TDYTLTISSLQPEDFATYYCLQTTHLYTFGCGTKLEIK [SEQ ID NO: 55] |
| | DVQMTQSPSSLSASVGDRVTITCKSSQSLLHSNGKTYLNWYLQQKPGQAPKLLLYLVSKLESGVPSRFSGS |
| | GSGTDYTLTISSLQPEDFATYYCLQTTHLYTFGCGTKLEIKGGGGSGGGGSGGGGSGGGGSQVQLVQSGAE |
| | VKKPGASVKVSCKASGFNIKDDYIHWVRQAPGQCLEWMGWIDSENGDTEYASKFQGRVTITADTSANTAYM |
| | ELSSLRSEDTAVYYCATSEYYSYDLFVYWGQGTLVTVSS [SEQ ID NO: 56] |
| Exemplary | CAGGTGCAGCTGGTGCAGAGCGGCGCCGAGGTGAAGAAGCCCGGCGCCAGCGTGAAGGTGAGCTGCAAGGC |
| nucleotide | CAGCGGCTTCAACATCAAGGACGACTACATCCACTGGGTGAGGCAGGCCCCCGGCCAGTGCCTGGAGTGGA |
| sequence of | TGGGCTGGATCGACAGCGAGAACGGCGACACCGAGTACGCCAGCAAGTTCCAGGGCAGGGTGACCATCACC |
| h5E7-YD-C6 | GCCGACACCAGCGCCAACACCGCCTACATGGAGCTGAGCAGCCTGAGGAGCGAGGACACCGCCGTGTACTA |
| scFv | CTGCGCCACCAGCGAGTACTACAGCTACGACCTGTTCGTGTACTGGGGCCAGGGCACCCTGGTGACCGTGA |
| | GCAGCGGCGGCGGCGGCAGCGGCGGCGGCGGCAGCGGCGGCGGCGGCAGCGGCGGCGGCGGCAGCGACGTG |
| | CAGATGACCCAGAGCCCCAGCAGCCTGAGCGCCAGCGTGGGCGACAGGGTGACCATCACCTGCAAGAGCAG |
| | CCAGAGCCTGCTGCACAGCAACGGCAAGACCTACCTGAACTGGCTGCAGCAGAAGCCCGGCCAGGCCCCCA |
| | AGCTGCTGCTGTACCTGGTGAGCAAGCTGGAGAGCGGCGTGCCCAGCAGGTTCAGCGGCAGCGGCAGCGGC |
| | ACCGACTACACCCTGACCATCAGCAGCCTGCAGCCCGAGGACTTCGCCACCTACTACTGCCTGCAGACCAC |
| | CCACCTGTACACCTTCGGCTGCGGCACCAAGCTGGAGATCAAG [SEQ ID NO: 57] |
| | GACGTGCAGATGACCCAGAGCCCCAGCAGCCTGAGCGCCAGCGTGGGCGACAGGGTGACCATCACCTGCAA |
| | GAGCAGCCAGAGCCTGCTGCACAGCAACGGCAAGACCTACCTGAACTGGCTGCAGCAGAAGCCCGGCCAGG |
| | CCCCCAAGCTGCTGCTGTACCTGGTGAGCAAGCTGGAGAGCGGCGTGCCCAGCAGGTTCAGCGGCAGCGGC |
| | AGCGGCACCGACTACACCCTGACCATCAGCAGCCTGCAGCCCGAGGACTTCGCCACCTACTACTGCCTGCA |
| | GACCACCCACCTGTACACCTTCGGCTGCGGCACCAAGCTGGAGATCAAGGGCGGCGGCGGCAGCGGCGGCG |
| | GCGGCAGCGGCGGCGGCGGCAGCGGCGGCGGCGGCAGCCAGGTGCAGCTGGTGCAGAGCGGCGCCGAGGTG |
| | AAGAAGCCCGGCGCCAGCGTGAAGGTGAGCTGCAAGGCCAGCGGCTTCAACATCAAGGACGACTACATCCA |
| | CTGGGTGAGGCAGGCCCCCGGCCAGTGCCTGGAGTGGATGGGCTGGATCGACAGCGAGAACGGCGACACCG |
| | AGTACGCCAGCAAGTTCCAGGGCAGGGTGACCATCACCGCCGACACCAGCGCCAACACCGCCTACATGGAG |
| | CTGAGCAGCCTGAGGAGCGAGGACACCGCCGTGTACTACTGCGCCACCAGCGAGTACTACAGCTACGACCT |
| | GTTCGTGTACTGGGGCCAGGGCACCCTGGTGACCGTGAGCAGC [SEQ ID NO: 58] |
| svFv of | QVQLVQSGAEVKKPGASVKVSCKASGFNIKDDYIHWVRQAPGQCLEWMGWIDSENGDTEYASKFQGRVTIT |
| h5E7-YD-F3 | ADTSANTAYMELSSLRSEDTAVYYCATSSYWSYDLLVYWGQGTLVTVSSGGGGSGGGGSGGGGSGGGGSDV |
| | QMTQSPSSLSASVGDRVTITCKSSQSLLHSNGKTYLNWLQQKPGQAPKLLLYLVSKLESGVPSRFSGSGSG |
| | TDYTLTISSLQPEDFATYYCLQTTHLYTFGCGTKLEIK [SEQ ID NO: 59] |
| | DVQMTQSPSSLSASVGDRVTITCKSSQSLLHSNGKTYLNWLQQKPGQAPKLLLYLVSKLESGVPSRFSGSG |
| | SGTDYTLTISSLQPEDFATYYCLQTTHLYTFGCGTKLEIKGGGGSGGGGSGGGGSGGGGSQVQLVQSGAEV |
| | KKPGASVKVSCKASGFNIKDDYIHWVRQAPGQCLEWMGWIDSENGDTEYASKFQGRVTITADTSANTAYME |
| | LSSLRSEDTAVYYCATSSYWSYDLLVYWGQGTLVTVSS [SEQ ID NO: 60] |
| Exemplary | CAGGTGCAGCTGGTGCAGAGCGGCGCCGAGGTGAAGAAGCCCGGCGCCAGCGTGAAGGTGAGCTGCAAGGC |
| nucleotide | CAGCGGCTTCAACATCAAGGACGACTACATCCACTGGGTGAGGCAGGCCCCCGGCCAGTGCCTGGAGTGGA |
| sequence of | TGGGCTGGATCGACAGCGAGAACGGCGACACCGAGTACGCCAGCAAGTTCCAGGGCAGGGTGACCATCACC |
| h5E7-YD-F3 | GCCGACACCAGCGCCAACACCGCCTACATGGAGCTGAGCAGCCTGAGGAGCGAGGACACCGCCGTGTACTA |
| scFv | CTGCGCCACCAGCAGCTACTGGAGCTACGACCTGCTGGTGTACTGGGGCCAGGGCACCCTGGTGACCGTGA |
| | GCAGCGGCGGCGGCGGCAGCGGCGGCGGCGGCAGCGGCGGCGGCGGCAGCGGCGGCGGCGGCAGCGACGTG |
| | CAGATGACCCAGAGCCCCAGCAGCCTGAGCGCCAGCGTGGGCGACAGGGTGACCATCACCTGCAAGAGCAG |
| | CCAGAGCCTGCTGCACAGCAACGGCAAGACCTACCTGAACTGGCTGCAGCAGAAGCCCGGCCAGGCCCCCA |
| | AGCTGCTGCTGTACCTGGTGAGCAAGCTGGAGAGCGGCGTGCCCAGCAGGTTCAGCGGCAGCGGCAGCGGC |
| | ACCGACTACACCCTGACCATCAGCAGCCTGCAGCCCGAGGACTTCGCCACCTACTACTGCCTGCAGACCAC |
| | CCACCTGTACACCTTCGGCTGCGGCACCAAGCTGGAGATCAAG [SEQ ID NO: 61] |
| | GACGTGCAGATGACCCAGAGCCCCAGCAGCCTGAGCGCCAGCGTGGGCGACAGGGTGACCATCACCTGCAA |
| | GAGCAGCCAGAGCCTGCTGCACAGCAACGGCAAGACCTACCTGAACTGGCTGCAGCAGAAGCCCGGCCAGG |
| | CCCCCAAGCTGCTGCTGTACCTGGTGAGCAAGCTGGAGAGCGGCGTGCCCAGCAGGTTCAGCGGCAGCGGC |
| | AGCGGCACCGACTACACCCTGACCATCAGCAGCCTGCAGCCCGAGGACTTCGCCACCTACTACTGCCTGCA |
| | GACCACCCACCTGTACACCTTCGGCTGCGGCACCAAGCTGGAGATCAAGGGCGGCGGCGGCAGCGGCGGCG |
| | GCGGCAGCGGCGGCGGCGGCAGCGGCGGCGGCGGCAGCCAGGTGCAGCTGGTGCAGAGCGGCGCCGAGGTG |
| | AAGAAGCCCGGCGCCAGCGTGAAGGTGAGCTGCAAGGCCAGCGGCTTCAACATCAAGGACGACTACATCCA |
| | CTGGGTGAGGCAGGCCCCCGGCCAGTGCCTGGAGTGGATGGGCTGGATCGACAGCGAGAACGGCGACACCG |
| | AGTACGCCAGCAAGTTCCAGGGCAGGGTGACCATCACCGCCGACACCAGCGCCAACACCGCCTACATGGAG |
| | CTGAGCAGCCTGAGGAGCGAGGACACCGCCGTGTACTACTGCGCCACCAGCAGCTACTGGAGCTACGACCT |
| | GCTGGTGTACTGGGGCCAGGGCACCCTGGTGACCGTGAGCAGC [SEQ ID NO: 62] |
| scFv of | QVLQVQSGAEVKKPGASVKVSCKASGFNIKDDYIHWVRQAPGQCLEWMGWIDSENGDTEYASKFQGRVTIT |
| h5E7-YD-A6 | ADTSANTAYMELSSLRSEDTAVYYCATSSYWSYDLFVYWGQGTLVTVSSGGGGSGGGGSGGGGSGGGGSDV |
| | QMTQSPSSLSASVGDRVTITCKSSQSLLHSNGKTYLNWLQQKPGQAPKLLLYLVSKLESGVPSRFSGSGSG |
| | TDYTLTISSLQPEDFATYYCLQTTHLYTFGCGTKLEIK [SEQ ID NO: 63] |
| | DVQMTQSPSSLSASVGDRVTITCKSSQSLLHSNGKTYLNWLQQKPGQAPKLLLYLVSKLESGVPSRFSGSG |
| | SGTDYTLTISSLQPEDFATYYCLQTTHLYTFGCGTKLEIKGGGGSGGGGSGGGGSGGGGSQVQLVQSGAEV |
| | KKPGASVKVSCKASGFNIKDDYIHWVRQAPGQCLEWMGWIDSENGDTEYASKFQGRVTITADTSANTAYME |
| | LSSLRSEDTAVYYCATSSYWSYDLFVYWGQGTLVTVSS [SEQ ID NO: 64] |
| Exemplary | CAGGTGCAGCTGGTGCAGAGCGGCGCCGAGGTGAAGAAGCCCGGCGCCAGCGTGAAGGTGAGCTGCAAGGC |
| nucleotide | CAGCGGCTTCAACATCAAGGACGACTACATCCACTGGGTGAGGCAGGCCCCCGGCCAGTGCCTGGAGTGGA |
| sequence of | TGGGCTGGATCGACAGCGAGAACGGCGACACCGAGTACGCCAGCAAGTTCCAGGGCAGGGTGACCATCACC |
| h5E7-YD-A6 | GCCGACACCAGCGCCAACACCGCCTACATGGAGCTGAGCAGCCTGAGGAGCGAGGACACCGCCGTGTACTA |
| scFv | CTGCGCCACCAGCAGCTACTGGAGCTACGACCTGTTCGTGTACTGGGGCCAGGGCACCCTGGTGACCGTGA |
| | GCAGCGGCGGCGGCGGCAGCGGCGGCGGCGGCAGCGGCGGCGGCGGCAGCGGCGGCGGCGGCAGCGACGTG |
| | CAGATGACCCAGAGCCCCAGCAGCCTGAGCGCCAGCGTGGGCGACAGGGTGACCATCACCTGCAAGAGCAG |
| | CCAGAGCCTGCTGCACAGCAACGGCAAGACCTACCTGAACTGGCTGCAGCAGAAGCCCGGCCAGGCCCCCA |
| | AGCTGCTGCTGTACCTGGTGAGCAAGCTGGAGAGCGGCGTGCCCAGCAGGTTCAGCGGCAGCGGCAGCGGC |
| | ACCGACTACACCCTGACCATCAGCAGCCTGCAGCCCGAGGACTTCGCCACCTACTGACTGCCTGCAGACCA |
| | CCCACCTGTACACCTTCGGCTGCGGCACCAAGCTGGAGATCAAG [SEQ ID NO: 65] |
| | GACTGTGCAGATGACCCAGAGCCCCAGCAGCCTGAGCGCCAGCGTGGGCGACAGGGTGACCATCACCTGCA |
| | AGAGCAGCCAGAGCCTGCTGCACAGCAACGGCAAGACCTACCTGAACTGGCTGCAGCAGAAGCCCGGCCAG |
| | GCCCCCAAGCTGCTGCTGTACCTGGTGAGCAAGCTGGAGAGCGGCGTGCCCAGCAGGTTCAGCGGCAGCGG |
| | CAGCGGCACCGACTACACCCTGACCATCAGCAGCCTGCAGCCCGAGGACTTCGCCACCTACTACTGCCTGC |
| | AGACCACCCACCTGTACACCTTCGGCTGCGGCACCAAGCTGGAGATCAAGGGCGGCGGCGGCAGCGGCGGC |
| | GGCGGCAGCGGCGGCGGCGGCAGCGGCGGCGGCGGCAGCCAGGTGCAGCTGGTGCAGAGCGGCGCCGAGGT |
| | GAAGAAGCCCGGCGCCAGCGTGAAGGTGAGCTGCAAGGCCAGCGGCTTCAACATCAAGGACGACTACATCC |
| | ACTGGGTGAGGCAGGCCCCCGGCCAGTGCCTGGAGTGGATGGGCTGGATCGACAGCGAGAACGGCGACACC |
| | GAGTACGCCAGCAAGTTCCAGGGCAGGGTGACCATCACCGCCGACACCAGCGCCAACACCGCCTACATGGA |
| | GCTGAGCAGCCTGAGGAGCGAGGACACCGCCGTGTACTACTGCGCCACCAGCAGCTACTGGAGCTACGACC |
| | TGTTCGTGTACTGGGGCCAGGGCACCCTGGTGACCGTGAGCAGC [SEQ ID NO: 66] |
| scFv of | QVQLVQSGAEVKKPGASVKVSCKASGFNIKDDYIHWVRQAPGQCLEWMGWIDSENGDTEYAKSFQGRVTIT |
| h5E7-YD-B5 | ADTSANTAYMELSSLRSEDTAVYYCATSTYWSYDLFVYWGQGTLVTVSSGGGGSGGGGSGGGGSGGGGSDV |
| | QMTQSPSSLSASVGDRVTITCKSSQSLLHSNGKTYLNWLQQKPGQAPKLLLYLVSKLESGVPSRFSGSGSG |
| | TDYTLTISSLQPEDFATYYCLQTTHLYTFGCGTKLEIK [SEQ ID NO: 67] |
| | DVQMTQSPSSLSASVGDRVTITCKSSQSLLHSNGKTYLNWLQQKPGQAPKLLLYLVSKLESGVPSRFSGSG |
| | SGTDYTLTISSLQPEDFATYYCLQTTHLYTFGCGTKLEIKGGGGSGGGGSGGGGSGGGGSQVQLVQSGAEV |
| | KKPGASVKVSCKASGFNIKDDYIHWVRQAPGQCLEWMGWIDSENGDTEYASKFQGRVTITADTSANTAYME |
| | LSSLRSEDTAVYYCATSTYWSYDLFVYWGQGTLVTVSS [SEQ ID NO: 68] |
| Exemplary | CAGGTGCAGCTGGTGCAGAGCGGCGCCGAGGTGAAGAAGCCCGGCGCCAGCGTGAAGGTGAGCTGCAAGGC |
| nucleotide | CAGCGGCTTCAACATCAAGGACGACTACATCCACTGGGTGAGGCAGGCCCCCGGCCAGTGCCTGGAGTGGA |
| sequence of | TGGGCTGGATCGACAGCGAGAACGGCGACACCGAGTACGCCAGCAAGTTCCAGGGCAGGGTGACCATCACC |
| h5E7-YD-B5 | GCCGACACCAGCGCCAACACCGCCTACATGGAGCTGAGCAGCCTGAGGAGCGAGGACACCGCCGTGTACTA |
| scFv | CTGCGCCACCAGCACCTACTGGAGCTACGACCTGTTCGTGTACTGGGGCCAGGGCACCCTGGTGACCGTGA |
| | GCAGCGGCGGCGGCGGCAGCGGCGGCGGCGGCAGCGGCGGCGGCGGCAGCGGCGGCGGCGGCAGCGACGTG |
| | CAGATGACCCAGAGCCCCAGCAGCCTGAGCGCCAGCGTGGGCGACAGGGTGACCATCACCTGCAAGAGCAG |
| | CCAGAGCCTGCTGCACAGCAACGGCAAGACCTACCTGAACTGGCTGCAGCAGAAGCCCGGCCAGGCCCCCA |
| | AGCTGCTGCTGTACCTGGTGAGCAAGCTGGAGAGCGGCGTGCCCAGCAGGTTCAGCGGCAGCGGCAGCGGC |
| | ACCGACTACACCCTGACCATCAGCAGCCTGCAGCCCGAGGACTTCGCCACCTACTACTGCCTGCAGACCAC |
| | CCACCTGTACACCTTCGGCTGCGGCACCAAGCTGGAGATCAAG [SEQ ID NO: 69] |
| | GACGTGCAGATGACCCAGAGCCCCAGCAGCCTGAGCGCCAGCGTGGGCGACAGGGTGACCATCACCTGCAA |
| | GAGCAGCCAGAGCCTGCTGCACAGCAACGGCAAGACCTACCTGAACTGGCTGCAGCAGAAGCCCGGCCAGG |
| | CCCCCAAGCTGCTGCTGTACCTGGTGAGCAAGCTGGAGAGCGGCGTGCCCAGCAGGTTCAGCGGCAGCGGC |
| | AGCGGCACCGACTACACCCTGACCATCAGCAGCCTGCAGCCCGAGGACTTCGCCACCTACTACTGCCTGCA |
| | GACCACCCACCTGTACACCTTCGGCTGCGGCACCAAGCTGGAGATCAAGGGCGGCGGCGGCAGCGGCGGCG |
| | GCGGCAGCGGCGGCGGCGGCAGCGGCGGCGGCGGCAGCCAGGTGCAGCTGGTGCAGAGCGGCGCCGAGGTG |
| | AAGAAGCCCGGCGCCAGCGTGAAGGTGAGCTGCAAGGCCAGCGGCTTCAACATCAAGGACGACTACATCCA |
| | CTGGGTGAGGCAGGCCCCCGGCCAGTGCCTGGAGTGGATGGGCTGGATCGACAGCGAGAACGGCGACACCG |
| | AGTACGCCAGCAAGTTCCAGGGCAGGGTGACCATCACCGCCGACACCAGCGCCAACACCGCCTACATGGAG |
| | CTGAGCAGCCTGAGGAGCGAGGACACCGCCGTGTACTACTGCGCCACCAGCACCTACTGGAGCTACGACCT |
| | GTTCGTGTACTGGGGCCAGGGCACCCTGGTGACCGTGAGCAGC [SEQ ID NO: 70] |

An Antigen-Binding Site that Binds an Epitope on the Extracellular Domain of Human DLL3

The invention provides a DLL3 antigen-binding site that binds an epitope on the ECD of human DLL3. In some embodiments, the DLL3 antigen-binding site binds to an epitope which includes the N-terminus of the ECD. In some embodiments, the DLL3 antigen-binding site binds to an epitope which includes the DSL of the ECD. In some embodiments, the DLL3 antigen-binding site binds to an epitope which includes the EGF1 of the ECD. In some embodiments, the DLL3 antigen-binding site binds to an epitope which includes the EGF2 of the ECD. In some embodiments, the DLL3 antigen-binding site binds to an epitope which includes the EGF3 of the ECD. In some embodiments, the DLL3 antigen-binding site binds to an epitope which includes the EGF4 of the ECD. In some embodiments, the DLL3 antigen-binding site binds to an epitope which includes the EGF5 of the ECD. In some embodiments, the DLL3 antigen-binding site binds to an epitope which includes the EGF6 of the ECD.

The invention also provides a DLL3 antigen-binding site that binds the ECD of human DLL3 with high affinity, and with little cross-reactivity to human DLL1 and DLL4. In certain embodiments, the DLL3 antigen-binding site binds DLL3 with a K_{D} of 0.001 nM-10 nM, e.g., 0.001 nM-9 nM, 0.001 nM-8 nM, 0.001 nM-7 nM, 0.001 nM-6 nM, 0.001 nM-5 nM, 0.001 nM-4 nM, 0.001 nM-3 nM, 0.001 nM-2 nM, 0.001 nM-1 nM, 0.001 nM-0.9 nM, 0.001 nM-0.8 nM, 0.001 nM-0.7 nM, 0.001 nM-0.6 nM, 0.001 nM-0.5 nM, 0.001 nM-0.4 nM, 0.001 nM-0.3 nM, 0.001 nM-0.2 nM, 0.001 nM-0.1 nM, 0.05 nM-10 nM, 0.1 nM-10 nM, 0.2 nM-10 nM, 0.3 nM-10 nM, 0.4 nM-10 nM, 0.5 nM-10 nM, 1 nM-10 nM, 2 nM-10 nM, 3 nM-10 nM, 4 nM-10 nM, 5 nM-10 nM, 6 nM-10 nM, 7 nM-10 nM, 8 nM-10 nM, or 9 nM-10 nM, as measured using surface plasmon resonance. In some embodiments, the DLL3 antigen-binding site binds DLL3 with a K_{D} of <0.011 nM, about 0.203 nM, about 0.669 nM, about 0.184 nM, about 1.12 nM, about 1.92 nM, about 5.11 nM, about 6.1 nM, or about 8.44 nM, as measured using surface plasmon resonance.

In another aspect, the invention provides an antibody which both incorporates one of the DLL3 antigen-binding sites described herein and displays high thermostability. In some embodiments, the melting temperature of the antibody is equal to or above 70° C., e.g., 70° C., 71° C., 72° C. 73° C., 74° C., 75° C., 76° C., 77° C., 78° C., 79° C., 80° C., 81° C., 82° C., 83° C., or 84° C.

In some embodiments, the DLL3 antigen-binding site described herein includes a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:39. The heavy chain variable domain can include amino acid sequences of SEQ ID NO:40 as CDR1, SEQ ID NO:41 as CDR2, and SEQ ID NO:50 as CDR3 of SEQ ID NO:39. In some embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:39 is combined with a light chain variable domain to form an antigen-binding site capable of binding to DLL3. For example, an antibody heavy chain variable domain at least 90% (e.g., 91%, 92%, 93%, 94%, 95%, 96%, 97%, 98%, 99%, or 100%) identical to the amino acid sequence of SEQ ID NO:39 can be paired with an antibody light chain variable domain at least 90% (e.g., 91%, 92%, 93%, 94%, 95%, 96%, 97%, 98%, 99%, or 100%) identical to the amino acid sequence of SEQ ID NO:34. The antibody light chain variable domain at least 90% (e.g., 91%, 92%, 93%, 94%, 95%, 96%, 97%, 98%, 99%, or 100%) identical to the amino acid sequence of SEQ ID NO:34 can include amino acid sequences SEQ ID NO:43 as CDR1, SEQ ID NO:44 as CDR2, and SEQ ID NO:45 as CDR3 of SEQ ID NO:34.

In some embodiments, the DLL3 antigen-binding site described herein includes a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:33. The heavy chain variable domain can include amino acid sequences of SEQ ID NO:40 as CDR1, SEQ ID NO:41 as CDR2, and SEQ ID NO:42 as CDR3 of SEQ ID NO:33. In some embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:33 is combined with a light chain variable domain to form an antigen-binding site capable of binding to DLL3. For example, an antibody heavy chain variable domain at least 90% (e.g., 91%, 92%, 93%, 94%, 95%, 96%, 97%, 98%, 99%, or 100%) identical to the amino acid sequence of SEQ ID NO:33 can be paired with an antibody light chain variable domain at least 90% (e.g., 91%, 92%, 93%, 94%, 95%, 96%, 97%, 98%, 99%, or 100%) identical to the amino acid sequence of SEQ ID NO:34. The antibody light chain variable domain at least 90% (e.g., 91%, 92%, 93%, 94%, 95%, 96%, 97%, 98%, 99%, or 100%) identical to the amino acid sequence of SEQ ID NO:34 can include amino acid sequences SEQ ID NO:43 as CDR1, SEQ ID NO:44 as CDR2, and SEQ ID NO:45 as CDR3 of SEQ ID NO:34.

In some embodiments, the DLL3 antigen-binding site described herein includes a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:35. The heavy chain variable domain can include amino acid sequences of SEQ ID NO:40 as CDR1, SEQ ID NO:41 as CDR2, and SEQ ID NO:46 as CDR3 of SEQ ID NO:35. In some embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:35 is combined with a light chain variable domain to form an antigen-binding site capable of binding to DLL3. For example, an antibody heavy chain variable domain at least 90% (e.g., 91%, 92%, 93%, 94%, 95%, 96%, 97%, 98%, 99%, or 100%) identical to the amino acid sequence of SEQ ID NO:35 can be paired with an antibody light chain variable domain at least 90% (e.g., 91%, 92%, 93%, 94%, 95%, 96%, 97%, 98%, 99%, or 100%) identical to the amino acid sequence of SEQ ID NO:34. The antibody light chain variable domain at least 90% (e.g., 91%, 92%, 93%, 94%, 95%, 96%, 97%, 98%, 99%, or 100%) identical to the amino acid sequence of SEQ ID NO:34 can include amino acid sequences SEQ ID NO:43 as CDR1, SEQ ID NO:44 as CDR2, and SEQ ID NO:45 as CDR3 of SEQ ID NO:34.

In some embodiments, the DLL3 antigen-binding site described herein includes a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:36. The heavy chain variable domain can include amino acid sequences of SEQ ID NO:40 as CDR1, SEQ ID NO:41 as CDR2, and SEQ ID NO:47 as CDR3 of SEQ ID NO:36. In some embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:36 is combined with a light chain variable domain to form an antigen-binding site capable of binding to DLL3. For example, an antibody heavy chain variable domain at least 90% (e.g., 91%, 92%, 93%, 94%, 95%, 96%, 97%, 98%, 99%, or 100%) identical to the amino acid sequence of SEQ ID NO:36 can be paired with an antibody light chain variable domain at least 90% (e.g., 91%, 92%, 93%, 94%, 95%, 96%, 97%, 98%, 99%, or 100%) identical to the amino acid sequence of SEQ ID NO:34. The antibody light chain variable domain at least 90% (e.g., 91%, 92%, 93%, 94%, 95%, 96%, 97%, 98%, 99%, or 100%) identical to the amino acid sequence of SEQ ID NO:34 can include amino acid sequences SEQ ID NO:43 as CDR1, SEQ ID NO:44 as CDR2, and SEQ ID NO:45 as CDR3 of SEQ ID NO:34.

In some embodiments, the DLL3 antigen-binding site described herein includes a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:37. The heavy chain variable domain can include amino acid sequences of SEQ ID NO:40 as CDR1, SEQ ID NO:41 as CDR2, and SEQ ID NO:48 as CDR3 of SEQ ID NO:37. In some embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:37 is combined with a light chain variable domain to form an antigen-binding site capable of binding to DLL3. For example, an antibody heavy chain variable domain at least 90% (e.g., 91%, 92%, 93%, 94%, 95%, 96%, 97%, 98%, 99%, or 100%) identical to the amino acid sequence of SEQ ID NO:37 can be paired with an antibody light chain variable domain at least 90% (e.g., 91%, 92%, 93%, 94%, 95%, 96%, 97%, 98%, 99%, or 100%) identical to the amino acid sequence of SEQ ID NO:34. The antibody light chain variable domain at least 90% (e.g., 91%, 92%, 93%, 94%, 95%, 96%, 97%, 98%, 99%, or 100%) identical to the amino acid sequence of SEQ ID NO:34 can include amino acid sequences SEQ ID NO:43 as CDR1, SEQ ID NO:44 as CDR2, and SEQ ID NO:45 as CDR3 of SEQ ID NO:34.

In some embodiments, the DLL3 antigen-binding site described herein includes a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:38. The heavy chain variable domain can include amino acid sequences of SEQ ID NO:40 as CDR1, SEQ ID NO:41 as CDR2, and SEQ ID NO:49 as CDR3 of SEQ ID NO:38. In some embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:38 is combined with a light chain variable domain to form an antigen-binding site capable of binding to DLL3. For example, an antibody heavy chain variable domain at least 90% (e.g., 91%, 92%, 93%, 94%, 95%, 96%, 97%, 98%, 99%, or 100%) identical to the amino acid sequence of SEQ ID NO:38 can be paired with an antibody light chain variable domain at least 90% (e.g., 91%, 92%, 93%, 94%, 95%, 96%, 97%, 98%, 99%, or 100%) identical to the amino acid sequence of SEQ ID NO:34. The antibody light chain variable domain at least 90% (e.g., 91%, 92%, 93%, 94%, 95%, 96%, 97%, 98%, 99%, or 100%) identical to the amino acid sequence of SEQ ID NO:34 can include amino acid sequences SEQ ID NO:43 as CDR1, SEQ ID NO:44 as CDR2, and SEQ ID NO:45 as CDR3 of SEQ ID NO:34.

In some embodiments, the DLL3 antigen-binding site described herein includes a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:1. The heavy chain variable domain can include amino acid sequences of SEQ ID NO:9 as CDR1, SEQ ID NO:10 as CDR2, and SEQ ID NO:11 as CDR3 of SEQ ID NO:1. In some embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:1 is combined with a light chain variable domain to form an antigen-binding site capable of binding to DLL3. For example, an antibody heavy chain variable domain at least 90% (e.g., 91%, 92%, 93%, 94%, 95%, 96%, 97%, 98%, 99%, or 100%) identical to the amino acid sequence of SEQ ID NO:1 can be paired with an antibody light chain variable domain at least 90% (e.g., 91%, 92%, 93%, 94%, 95%, 96%, 97%, 98%, 99%, or 100%) identical to the amino acid sequence of SEQ ID NO:2. The antibody light chain variable domain at least 90% (e.g., 91%, 92%, 93%, 94%, 95%, 96%, 97%, 98%, 99%, or 100%) identical to the amino acid sequence of SEQ ID NO:2 can include amino acid sequences SEQ ID NO:12 as CDR1, SEQ ID NO:13 as CDR2, and SEQ ID NO:14 as CDR3 of SEQ ID NO:2.

In some embodiments, the DLL3 antigen-binding site described herein includes a heavy chain variable domain including an amino acid sequence at least 90% (e.g., 91%, 92%, 93%, 94%, 95%, 96%, 97%, 98%, 99%, or 100%) identical to the amino acid sequence of SEQ ID NO:3. The heavy chain variable domain can include amino acid sequences of SEQ ID NO:15 as CDR1, SEQ ID NO:16 as CDR2, and SEQ ID NO:17 as CDR3 of SEQ ID NO:3. In some embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:3 is combined with a light chain variable domain to form an antigen-binding site capable of binding to DLL3. For example, an antibody heavy chain variable domain at least 90% (e.g., 91%, 92%, 93%, 94%, 95%, 96%, 97%, 98%, 99%, or 100%) identical to the amino acid sequence of SEQ ID NO:3 can be paired with an antibody light chain variable domain at least 90% (e.g., 91%, 92%, 93%, 94%, 95%, 96%, 97%, 98%, 99%, or 100%) identical to the amino acid sequence of SEQ ID NO:4. The antibody light chain variable domain at least 90% (e.g., 91%, 92%, 93%, 94%, 95%, 96%, 97%, 98%, 99%, or 100%) identical to the amino acid sequence of SEQ ID NO:2 can include amino acid sequences SEQ ID NO:18 as CDR1, SEQ ID NO:19 as CDR2, and SEQ ID NO:20 as CDR3 of SEQ ID NO:4.

In some embodiments, the DLL3 antigen-binding site described herein includes a heavy chain variable domain including an amino acid sequence at least 90% (e.g., 91%, 92%, 93%, 94%, 95%, 96%, 97%, 98%, 99%, or 100%) identical to the amino acid sequence of SEQ ID NO:5. The heavy chain variable domain can include amino acid sequences of SEQ ID NO:21 as CDR1, SEQ ID NO:22 CDR2, and SEQ ID NO:23 CDR3 of SEQ ID NO:5. In some embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:5 is combined with a light chain variable domain to form an antigen-binding site capable of binding to DLL3. For example, an antibody heavy chain variable domain at least 90% (e.g., 91%, 92%, 93%, 94%, 95%, 96%, 97%, 98%, 99%, or 100%) identical to the amino acid sequence of SEQ ID NO:5 can be paired with an antibody light chain variable domain at least 90% (e.g., 91%, 92%, 93%, 94%, 95%, 96%, 97%, 98%, 99%, or 100%) identical to the amino acid sequence of SEQ ID NO:6. The antibody light chain variable domain at least 90% (e.g., 91%, 92%, 93%, 94%, 95%, 96%, 97%, 98%, 99%, or 100%) identical to the amino acid sequence of SEQ ID NO:6 can include amino acid sequences SEQ ID NO:24 as CDR1, SEQ ID NO:25 as CDR2, and SEQ ID NO:26 as CDR3 of SEQ ID NO:6.

In some embodiments, the DLL3 antigen-binding site described herein includes a heavy chain variable domain including an amino acid sequence at least 90% (e.g., 91%, 92%, 93%, 94%, 95%, 96%, 97%, 98%, 99%, or 100%) identical to the amino acid sequence of SEQ ID NO:7. The heavy chain variable domain can include amino acid sequences of SEQ ID NO:27 as CDR1, SEQ ID NO:28 as CDR2, and SEQ ID NO:29 as CDR3 of SEQ ID NO:7. In some embodiments, the antibody heavy chain variable domain which includes an amino acid sequence at least 90% (e.g., 91%, 92%, 93%, 94%, 95%, 96%, 97%, 98%, 99%, or 100%) identical to the amino acid sequence of SEQ ID NO:7 is combined with a light chain variable domain to form an antigen-binding site capable of binding to DLL3. For example, an antibody heavy chain variable domain at least 90% (e.g., 91%, 92%, 93%, 94%, 95%, 96%, 97%, 98%, 99%, or 100%) identical to the amino acid sequence of SEQ ID NO:7 can be paired with an antibody light chain variable domain at least 90% (e.g., 91%, 92%, 93%, 94%, 95%, 96%, 97%, 98%, 99%, or 100%) identical to the amino acid sequence of SEQ ID NO:8. The antibody light chain variable domain at least 90% (e.g., 91%, 92%, 93%, 94%, 95%, 96%, 97%, 98%, 99%, or 100%) identical to the amino acid sequence of SEQ ID NO:8 can include amino acid sequences SEQ ID NO:30 as CDR1, SEQ ID NO:31 as CDR2, and SEQ ID NO:32 as CDR3 of SEQ ID NO:8.

In some embodiments, the DLL3 antigen-binding sites described herein bind EGF4, but not the N-terminus, EGF5 or EGF6. These DLL3 antigen-binding sites can include a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:5, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:6. In some embodiments, the DLL3 antigen-binding sites that include a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:5 and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:6, can incorporate amino acid sequences of SEQ ID NO:21 as CDR1, SEQ ID NO:22 as CDR2, and SEQ ID NO:23 as CDR3 of SEQ ID NO:5, and amino acid sequences SEQ ID NO:24 as CDR1, SEQ ID NO:25 as CDR2, and SEQ ID NO:26 as CDR3 of SEQ ID NO:6.

### A Heavy Chain Including the DLL3 Antigen-Binding Sites

Each of the antibody heavy chain variable domains having amino acid sequences 90% identical to SEQ ID NOs: 1, 3, 5, or 7 described herein can optionally be coupled to an amino acid sequence at least 90% identical to an antibody constant region, such as an IgG constant region including hinge, CH2 and CH3 domains with or without a CH1 domain. In some embodiments, the amino acid sequence of the constant region is at least 90% identical to a human antibody constant region, such as an human IgG1 constant region, IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

### II. Multi-Specific Binding Proteins

In some embodiments, the present invention provides a protein that includes a human DLL3 antigen-binding site incorporating a heavy chain variable domain having an amino acid sequence at least 90% identical to the amino acid sequence of SEQ ID NO: 1, 3, 5, or 7, wherein the protein further includes a second antigen-binding site. In some embodiments, the second antigen-binding site is the same as the first DLL3 antigen-binding site. In some embodiments, the second antigen-binding site includes a heavy chain variable domain having an amino acid sequence at least 90% identical to the amino acid sequence of SEQ ID NO: 1, 3, 5, or 7. In some embodiments, the second antigen-binding site binds to an antigen different from DLL3.

In some embodiments, the protein bind to DLL3-expressing cancer cells, which can include but are not limited to small cell lung cancer, large cell neuroendocrine carcinoma, glioblastoma, Ewing's sarcoma, and cancers with neuroendocrine phenotype.

The protein can take various formats. For example, one format includes a first immunoglobulin heavy chain, a first immunoglobulin light chain, a second immunoglobulin heavy chain and a second immunoglobulin light chain. The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and a first light chain constant domain. The first immunoglobulin light chain, together with the first immunoglobulin heavy chain, forms an antigen-binding site that binds DLL3. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and a second light chain constant domain. The second immunoglobulin light chain, together with the second immunoglobulin heavy chain, forms the second antigen-binding site (FIG. 9).

Another exemplary format involves a first immunoglobulin heavy chain, a second immunoglobulin heavy chain and an immunoglobulin light chain (FIG. 10). The first immunoglobulin heavy chain includes a first Fc (hinge-CH2-CH3) domain fused via either a linker or an antibody hinge to a single-chain variable fragment (scFv) composed of a heavy variable domain and light chain variable domain which pair and bind DLL3. The second immunoglobulin heavy chain includes a second Fc (hinge-CH2-CH3) domain, a second heavy chain variable domain and optionally a CH1 heavy chain domain. The immunoglobulin light chain includes a light chain variable domain and a light chain constant domain. The second immunoglobulin heavy chain pairs with the immunoglobulin light chain and binds the second antigen (FIG. 10).

One or more additional binding motifs may be fused to the C-terminus of the constant region CH3 domain, optionally via a linker sequence. In certain embodiments, the additional antigen-binding site could be a single-chain or disulfide-stabilized variable region (scFv) or could form a tetravalent or trivalent molecule.

In some embodiments, the protein is in the Triomab form, which is a trifunctional, bispecific antibody that maintains an IgG-like shape. This chimera consists of two half antibodies, each with one light and one heavy chain, that originate from two parental antibodies.

In some embodiments, the protein is the KiH Common Light Chain (LC) form, which involves the knobs-into-holes (KIHs) technology. The KIH involves engineering C_{H}3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structured-guided phage library screening (Atwell S, Ridgway J B, Wells J A, Carter P., Stable heterodimers from remodeling the domain interface of a homodimer using a phage display library, J. Mol. Biol. (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc variant with improved affinity for FcγRs. Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the protein is in the dual-variable domain immunoglobulin (DVD-Ig™) form, which combines the target binding domains of two monoclonal antibodies via flexible naturally occurring linkers, and yields a tetravalent IgG-like molecule.

In some embodiments, the protein is in the Orthogonal Fab interface (Ortho-Fab) form. In the ortho-Fab IgG approach (Lewis S M, Wu X, Pustilnik A, Sereno A, Huang F, Rick H L, et al., Generation of bispecific IgG antibodies by structure-based design of an orthogonal Fab interface. Nat. Biotechnol. (2014) 32(2):191-8), structure-based regional design introduces complementary mutations at the LC and HC_{VH-CH1} interface in only one Fab, without any changes being made to the other Fab.

In some embodiments, the protein is in the 2-in-1 Ig format. In some embodiments, the multi-specific binding protein is in the ES form, which is a heterodimeric construct containing two different Fabs binding to targets 1 and target 2 fused to the Fc. Heterodimerization is ensured by electrostatic steering mutations in the Fc.

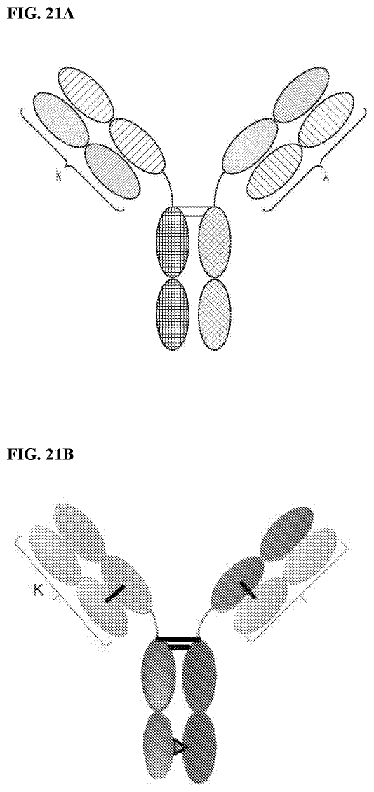

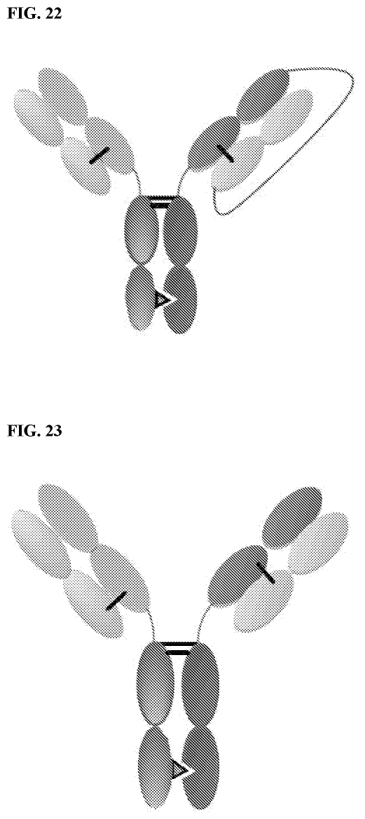

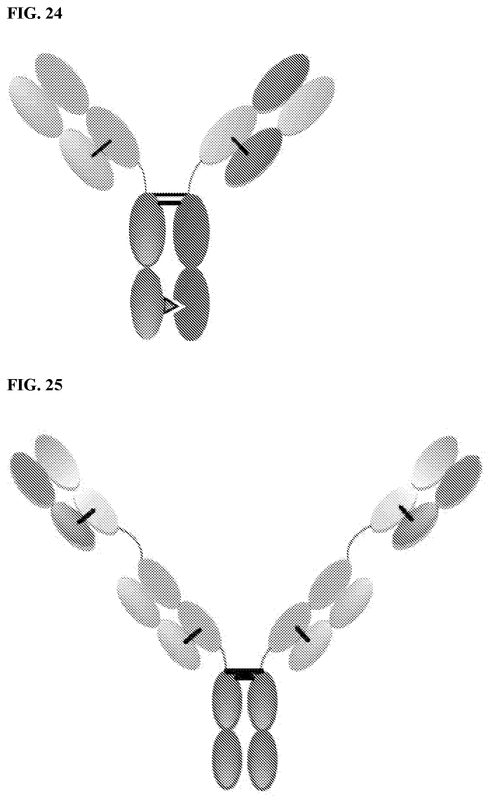

In some embodiments, the protein is in the κλ-Body form, which is a heterodimeric construct with two different Fabs fused to Fc stabilized by heterodimerization mutations: Fab1 targeting antigen 1 contains kappa LC, while second Fab targeting antigen 2 contains lambda LC. FIG. 21A is an exemplary representation of one form of a κλ-Body; FIG. 21B is an exemplary representation of another κλ-Body.

In some embodiments, the protein is in Fab Arm Exchange form (antibodies that exchange Fab arms by swapping a heavy chain and attached light chain (half-molecule) with a heavy-light chain pair from another molecule, which results in bispecific antibodies).

In some embodiments, the protein is in the SEED Body form. The strand-exchange engineered domain (SEED) platform was designed to generate asymmetric and bispecific antibody-like molecules, a capability that expands therapeutic applications of natural antibodies. This protein engineered platform is based on exchanging structurally related sequences of immunoglobulin within the conserved CH3 domains. The SEED design allows efficient generation of AG/GA heterodimers, while disfavoring homodimerization of AG and GA SEED CH3 domains. (Muda M. et al., Protein Eng. Des. Sel. (2011, 24(5):447-54)).

In some embodiments, the protein is in the LuZ-Y form, in which a leucine zipper is used to induce heterodimerization of two different HCs. (Wranik, B J. et al., J. Biol. Chem. (2012), 287:43331-9).

In some embodiments, the protein is in the Cov-X-Body form. In bispecific CovX-Bodies, two different peptides are joined together using a branched azetidinone linker and fused to the scaffold antibody under mild conditions in a site-specific manner. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V R et al., PNAS (2010), 107(52); 22611-22616).

In some embodiments, the protein is in an Oasc-Fab heterodimeric form that includes Fab binding to target 1, and scFab binding to target 2 fused to Fc. Heterodimerization is ensured by mutations in the Fc.

In some embodiments, the protein is in a DuetMab form, which is a heterodimeric construct containing two different Fabs binding to antigens 1 and 2, and Fc stabilized by heterodimerization mutations. Fab 1 and 2 contain differential S-S bridges that ensure correct LC and HC pairing.

In some embodiments, the protein is in a CrossmAb form, which is a heterodimeric construct with two different Fabs binding to targets 1 and 2, fused to Fc stabilized by heterodimerization. CL and CH1 domains and VH and VL domains are switched, e.g., CH1 is fused in-line with VL, while CL is fused in-line with VH.

In some embodiments, the protein is in a Fit-Ig form, which is a homodimeric construct where Fab binding to antigen 2 is fused to the N-terminus of HC of Fab that binds to antigen 1. The construct contains wild-type Fc.

In some embodiments, the protein described herein is an antibody, and has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In some embodiments, the amino acid sequence of the constant region is at least 90% identical to a human antibody constant region, such as an human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. In some embodiments, the antibody constant region chosen from an IgG constant region includes hinge, CH2 and CH3 domains with or without a CH1 domain. The antibody may have a light chain constant region chosen from, e.g., the (e.g., human) light chain constant regions of kappa or lambda.

The antibody constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments, the antibody has effector function and can fix complement. In some embodiments, the antibody does not recruit effector cells or fix complement. In some other embodiments, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In some embodiments, the antibody has a heavy chain constant region related to human IgG1 constant region, and mutations that can be incorporated into the CH1 of a human IgG1 constant region may be at amino acid V125, F126, P127, T135, T139, A140, F170, P171, and/or V173. In some embodiments, the antibody has a light chain constant region related to CK human IgG1 constant region, and mutations that can be incorporated into the CK of a human IgG1 constant region may be at amino acid E123, F116, S176, V163, S174, and/or T164.

In some embodiments, the antibody constant region includes an amino acid sequence at least 90% identical to human IgG1 constant region. In certain embodiments, the amino acid sequence of one polypeptide chain of the antibody constant region differs from the amino acid sequence of an IgG1 constant region at one or more positions selected from the group consisting of Q347, Y349, L351, S354, E356, E357, K360, Q362, S364, T366, L368, K370, K392, T394, D399, S400, D401, F405, Y407, K409, T411 and K439; and the amino acid sequence of the other polypeptide chain of the antibody constant region differs from the amino acid sequence of an IgG1 constant region at one or more positions selected from the group consisting of Q347, Y349, L351, S354, E356, E357, S364, T366, L368, K370, N390, K392, T394, D399, D401, F405, Y407, K409, T411 and K439.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In some embodiments, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

Alternatively, amino acid substitutions of the antibody constant region based on human IgG1 could be selected from the following sets of substitutions shown in Table 2.

TABLE 2

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions of the antibody constant region based on human IgG1 could be selected from the following sets of substitutions shown in Table 3.

TABLE 3

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions of the antibody constant region based on human IgG1 could be selected from the following sets of substitutions shown in Table 4.

TABLE 4

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, amino acid substitutions of the antibody constant region based on human IgG1 could be selected from the following sets of substitutions shown in Table 5.

TABLE 5

| First Polypeptide | Second Polypeptide |
|---|---|
| L351Y, D399R, D399K, S400K, | T366V, T366I, T366L, T366M, |
| S400R, Y407A, Y407I, Y407V | N390D, N390E, K392L, K392M, |
| | K392V, K392F K392D, K392E, |
| | K409F, K409W, T411D and T411E |

Alternatively, at least one amino acid substitution of the antibody constant region based on human IgG1 could be selected from the following sets of substitutions shown in Table 6, where the position(s) indicated in the First Polypeptide column is replaced by any known negatively-charged amino acid, and the position(s) indicated in the Second Polypeptide Column is replaced by any known positively-charged amino acid.

TABLE 6

| First Polypeptide | Second Polypeptide |
|---|---|
| K392, K370, K409, or K439 | D399, E356, or E357 |

Alternatively, at least one amino acid substitution of the antibody constant region based on human IgG1 could be selected from the following sets of substitutions shown in Table 7, where the position(s) indicated in the First Polypeptide column is replaced by any known positively-charged amino acid, and the position(s) indicated in the Second Polypeptide Column is replaced by any known negatively-charged amino acid.

TABLE 7

| First Polypeptide | Second Polypeptide |
|---|---|
| D399, E356, or E357 | K409, K439, K370, or K392 |

Alternatively, amino acid substitutions of the antibody constant region based on human IgG1 could be selected from the following sets of substitutions shown in Table 8.

TABLE 8

| First Polypeptide | Second Polypeptide |
|---|---|
| T350V, L351Y, F405A, and | T350V, T366L, K392L, and |
| Y407V | T394W |

Alternatively, or in addition, the structural stability of heterodimeric heavy chains within the antibody can be increased by introducing S354C on either of the first or second polypeptide chain, and Y349C on the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of T366, L368 and Y407.

In some embodiments, the amino acid sequence of one polypeptide chain of the antibody constant region differs from the amino acid sequence of an IgG1 constant region at one or more positions selected from the group consisting of T366, L368 and Y407, and wherein the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions selected from the group consisting of E357, K360, Q362, S364, L368, K370, T394, D401, F405, and T411 and wherein the amino acid sequence of the other polypeptide chain of the antibody constant region differs from the amino acid sequence of an IgG1 constant region at one or more positions selected from the group consisting of Y349, E357, S364, L368, K370, T394, D401, F405 and T411.

In some embodiments, the amino acid sequence of one polypeptide chain of the antibody constant region differs from the amino acid sequence of an IgG1 constant region at one or more positions selected from the group consisting of Y349, E357, S364, L368, K370, T394, D401, F405 and T411 and wherein the amino acid sequence of the other polypeptide chain of the antibody constant region differs from the amino acid sequence of an IgG1 constant region at one or more positions selected from the group consisting of E357, K360, Q362, S364, L368, K370, T394, D401, F405, and T411.

In some embodiments, the amino acid sequence of one polypeptide chain of the antibody constant region differs from the amino acid sequence of an IgG1 constant region at one or more positions selected from the group consisting of L351, D399, S400 and Y407 and wherein the amino acid sequence of the other polypeptide chain of the antibody constant region differs from the amino acid sequence of an IgG1 constant region at one or more positions selected from the group consisting of T366, N390, K392, K409 and T411.

In some embodiments, the amino acid sequence of one polypeptide chain of the antibody constant region differs from the amino acid sequence of an IgG1 constant region at one or more positions selected from the group consisting of T366, N390, K392, K409 and T411 and wherein the amino acid sequence of the other polypeptide chain of the antibody constant region differs from the amino acid sequence of an IgG1 constant region at one or more positions selected from the group consisting of L351, D399, S400 and Y407.

In some embodiments, the amino acid sequence of one polypeptide chain of the antibody constant region differs from the amino acid sequence of an IgG1 constant region at one or more positions selected from the group consisting of Q347, Y349, K360, and K409, and wherein the amino acid sequence of the other polypeptide chain of the antibody constant region differs from the amino acid sequence of an IgG1 constant region at one or more positions selected from the group consisting of Q347, E357, D399 and F405.

In some embodiments, the amino acid sequence of one polypeptide chain of the antibody constant region differs from the amino acid sequence of an IgG1 constant region at one or more positions selected from the group consisting of Q347, E357, D399 and F405, and wherein the amino acid sequence of the other polypeptide chain of the antibody constant region differs from the amino acid sequence of an IgG1 constant region at one or more positions selected from the group consisting of Y349, K360, Q347 and K409.

In some embodiments, the amino acid sequence of one polypeptide chain of the antibody constant region differs from the amino acid sequence of an IgG1 constant region at one or more positions selected from the group consisting of K370, K392, K409 and K439, and wherein the amino acid sequence of the other polypeptide chain of the antibody constant region differs from the amino acid sequence of an IgG1 constant region at one or more positions selected from the group consisting of D356, E357 and D399.

In some embodiments, the amino acid sequence of one polypeptide chain of the antibody constant region differs from the amino acid sequence of an IgG1 constant region at one or more positions selected from the group consisting of D356, E357 and D399, and wherein the amino acid sequence of the other polypeptide chain of the antibody constant region differs from the amino acid sequence of an IgG1 constant region at one or more positions selected from the group consisting of K370, K392, K409 and K439.

In some embodiments, the amino acid sequence of one polypeptide chain of the antibody constant region differs from the amino acid sequence of an IgG1 constant region at one or more positions selected from the group consisting of L351, E356, T366 and D399, and wherein the amino acid sequence of the other polypeptide chain of the antibody constant region differs from the amino acid sequence of an IgG1 constant region at one or more positions selected from the group consisting of Y349, L351, L368, K392 and K409.

In some embodiments, the amino acid sequence of one polypeptide chain of the antibody constant region differs from the amino acid sequence of an IgG1 constant region at one or more positions selected from the group consisting of Y349, L351, L368, K392 and K409, and wherein the amino acid sequence of the other polypeptide chain of the antibody constant region differs from the amino acid sequence of an IgG1 constant region at one or more positions selected from the group consisting of L351, E356, T366 and D399.

In some embodiments, the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

In some embodiments, the amino acid sequence of one polypeptide chain of the antibody constant region differs from the amino acid sequence of an IgG1 constant region by a Y349C substitution and wherein the amino acid sequence of the other polypeptide chain of the antibody constant region differs from the amino acid sequence of an IgG1 constant region by an S354C substitution.

In some embodiments, the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by Q347R, D399V and F405T substitutions.

In some embodiments, the amino acid sequence of one polypeptide chain of the antibody constant region differs from the amino acid sequence of an IgG1 constant region by Q347R, D399V and F405T substitutions and wherein the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitutions and wherein the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

The antibodies described herein can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the first immunoglobulin light chain can be cloned into a third expression vector; a fourth nucleic acid sequence encoding the second immunoglobulin light chain can be cloned into a fourth expression vector; the first, second, third and fourth expression vectors can be stably transfected together into host cells to produce the multimeric proteins.

To achieve the highest yield of antibodies, different ratios of the first, second, third and fourth expression vectors can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of antibodies. Antibodies can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

Competition assays for determining whether an antibody binds to the same epitope as, or competes for binding with a disclosed antibody, e.g., an antibody that includes a DLL3 antigen-binding site described above, are known in the art. Exemplary competition assays include immunoassays (e.g., ELISA assays, RIA assays), surface plasmon resonance (e.g., BIAcore analysis), bio-layer interferometry, and flow cytometry.

Typically, a competition assay involves the use of an antigen (e.g., a human DLL3 protein or fragment thereof) bound to a solid surface or expressed on a cell surface, a test DLL3-binding antibody and a reference antibody. The reference antibody is labeled and the test antibody is unlabeled. Competitive inhibition is measured by determining the amount of labeled reference antibody bound to the solid surface or cells in the presence of the test antibody. Usually the test antibody is present in excess (e.g., lx, 5×, 10×, 20× or 100×). Antibodies identified by competition assay (e.g., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated antibodies contain one or more somatic mutations. In these cases, non-human antibodies can be modified to a human germline sequence to optimize the antibody (e.g., by a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

A Protein Comprising an Antigen-Binding Site that Competes with the DLL3-Binding Sites Described Herein

In one aspect, the present invention provides a protein that includes an antigen-binding site that competes with the DLL3 antigen-binding sites described herein to bind to human DLL3.

In some embodiments, the present invention provides a protein that includes an antigen-binding site that competes for binding to human DLL3 with an antibody that includes an antibody heavy chain variable region having the amino acid sequence of SEQ ID NO:33 and an antibody light chain variable region having the amino acid sequence of SEQ ID NO:34.

In some embodiments, the present invention provides a protein that includes an antigen-binding site that competes for binding to human DLL3 with an antibody that includes an antibody heavy chain variable region having the amino acid sequence of SEQ ID NO:1 and an antibody light chain variable region having the amino acid sequence of SEQ ID NO:2.

In some embodiments, the present invention provides a protein that includes an antigen-binding site that competes for binding to human DLL3 with an antibody that includes an antibody heavy chain variable region having the amino acid sequence of SEQ ID NO:3 and an antibody light chain variable region having the amino acid sequence of SEQ ID NO:4.

In some embodiments, the present invention provides a protein that includes an antigen-binding site that competes for binding to human DLL3 with an antibody that includes an antibody heavy chain variable region having the amino acid sequence of SEQ ID NO:5 and an antibody light chain variable region having the amino acid sequence of SEQ ID NO:6.

In some embodiments, the present invention provides a protein that includes an antigen-binding site that competes for binding to human DLL3 with an antibody that includes an antibody heavy chain variable region having the amino acid sequence of SEQ ID NO:7 and an antibody light chain variable region having the amino acid sequence of SEQ ID NO:8.

In some embodiments, the protein that competes with the DLL3-binding antibody to bind to DLL3 described above includes an antigen-binding site having a heavy chain variable domain having an amino acid sequence at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:33 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:34. In some embodiments, the protein that competes with the DLL3-binding antibody to bind to DLL3 described above includes an antigen-binding site having a heavy chain variable domain having an amino acid sequence at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:1 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:2. In some embodiments, the protein that competes with the DLL3-binding antibody to bind to DLL3 described above includes an antigen-binding site having a heavy chain variable domain having an amino acid sequence at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:3 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:4. In some embodiments, the protein that competes with the DLL3-binding antibody to bind to DLL3 described above includes an antigen-binding site having a heavy chain variable domain having an amino acid sequence at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:5 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:6. In some embodiments, the protein that competes with the DLL3-binding antibody to bind to DLL3 described above includes an antigen-binding site having a heavy chain variable domain having an amino acid sequence at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:7 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:8. In some embodiments, the protein that competes with DLL3-binding antibody to bind to DLL3 described above includes an antigen-binding site which includes a heavy chain variable domain having an amino acid sequence at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:9 and a light chain variable domain having an amino acid sequence at least at least 50% (e.g., 50%, 60%, 70%, 80%, 90%, 91%, 92%, 93%, 94%, 95%, 96%, 97%, 98%, 99%, or 100%) identical to the amino acid sequence of SEQ ID NO:10.

### CAR T Cells, DLL3/CD3-Directed Bispecific T-Cell Engagers, Immunocytokines, Antibody-Drug Conjugates, and Immunotoxins

Another aspect of the present invention provides a molecule or complex comprising an antigen-binding site that binds DLL3 as disclosed herein. Exemplary molecules or complexes include but are not limited to chimeric antigen receptors (CARs), T-cell engagers (e.g., DLL3/CD3-directed bispecific T-cell engagers), immunocytokines, antibody-drug conjugates, and immunotoxins.

Any antigen-binding site that binds DLL3 as disclosed herein can be used, including but not limited to the antigen-binding site that binds DLL3 as disclosed in Section I. Antigen-Binding Site. In certain embodiments, the amino acid sequence(s) of the antigen-binding site that binds DLL3 are provided in Table 1. In certain embodiments, the antigen-binding site that binds DLL3 is an scFv. In certain embodiments, the scFv comprises an amino acid sequence at least 90% (e.g., 90%, 91%, 92%, 93%, 94%, 95%, 96%, 97%, 98%, 99%, or 100%) identical to an amino acid sequence selected from SEQ ID NO:51, SEQ ID NO:52, SEQ ID NO:55, SEQ ID NO:56, SEQ ID NO:59, SEQ ID NO:60, SEQ ID NO:63, SEQ ID NO:64, SEQ ID NO:67, and SEQ ID NO:68. In certain embodiments, the scFv comprises an amino acid sequence selected from SEQ ID NO:51, SEQ ID NO:52, SEQ ID NO:55, SEQ ID NO:56, SEQ ID NO:59, SEQ ID NO:60, SEQ ID NO:63, SEQ ID NO:64, SEQ ID NO:67, and SEQ ID NO:68.

### Chimeric Antigen Receptors (CARs)

In certain embodiments, the present invention provides a DLL3-targeting CAR comprising an antigen-binding site that binds DLL3 as disclosed herein (see, e.g., Table 1). The DLL3-targeting CAR can comprise an Fab fragment or an scFv.

The term “chimeric antigen receptor” or alternatively a “CAR” refers to a recombinant polypeptide construct comprising at least an extracellular antigen binding domain, a transmembrane domain and an intracellular signaling domain comprising a functional signaling domain derived from a stimulatory molecule (also referred to herein as a “primary signaling domain”).

Accordingly, in certain embodiments, the CAR comprises an extracellular antigen-binding site that binds DLL3 as disclosed herein, a transmembrane domain, and an intracellular signaling domain comprising a primary signaling domain. In certain embodiments, the CAR further comprises one or more functional signaling domains derived from at least one costimulatory molecule (also referred to as a “costimulatory signaling domain”).

In one embodiment, the CAR comprises a chimeric fusion protein comprising a DLL3-binding domain (e.g., DLL3-binding scFv domain) comprising CDR1, CDR2, and CDR3 of a heavy chain variable domain and CDR1, CDR2, and CDR3 of a light chain variable domain listed in Table 1 as an extracellular antigen binding domain, a transmembrane domain, and an intracellular signaling domain comprising a primary signaling domain. In one embodiment, the CAR comprises a chimeric fusion protein comprising a DLL3-binding domain (e.g., DLL3-binding scFv domain) comprising CDR1, CDR2, and CDR3 of a heavy chain variable domain and CDR1, CDR2, and CDR3 of a light chain variable domain listed in Table 1 as an extracellular antigen binding domain, a transmembrane domain and an intracellular signaling domain comprising a costimulatory signaling domain and a primary signaling domain. In one aspect, the CAR comprises a chimeric fusion protein comprising a DLL3-binding domain (e.g., DLL3-binding scFv domain) comprising CDR1, CDR2, and CDR3 of a heavy chain variable domain and CDR1, CDR2, and CDR3 of a light chain variable domain listed in Table 1 as an extracellular antigen binding domain, a transmembrane domain, and an intracellular signaling domain comprising two costimulatory signaling domains and a primary signaling domain. In one embodiment, the CAR comprises a chimeric fusion protein comprising a DLL3-binding domain comprising CDR1, CDR2, and CDR3 of a heavy chain variable domain and CDR1, CDR2, and CDR3 of a light chain variable domain listed in Table 1 as an extracellular antigen binding domain, a transmembrane domain, and an intracellular signaling domain comprising at least two costimulatory signaling domains and a primary signaling domain.

For example, in certain embodiments, the extracellular antigen binding domain comprises an antigen-binding site (e.g., an scFv) comprising a heavy chain variable domain comprising CDR1 having the amino acid sequence of SEQ ID NO:40, CDR2 having the amino acid sequence of SEQ ID NO:41, and CDR3 having the amino acid sequence of SEQ ID NO: 42, 46, 47, 48, 49, or 50, and a light chain variable domain comprising CDR1, CDR2, and CDR3 having the amino acid sequences of SEQ ID NOs: 43, 44, and 45, respectively. In certain embodiments, the heavy chain variable domain comprises an amino acid sequence at least 90% (e.g., 90%, 91%, 92%, 93%, 94%, 95%, 96%, 97%, 98%, 99%, or 100%) identical to SEQ ID NO:33, and the light chain variable domain comprises an amino acid sequence at least 90% (e.g., 90%, 91%, 92%, 93%, 94%, 95%, 96%, 97%, 98%, 99%, or 100%) identical to SEQ ID NO:34. In certain embodiments, the extracellular antigen binding domain comprises an scFv having the amino acid sequence of SEQ ID NO:51 or SEQ ID NO:52. In certain embodiments, the extracellular antigen binding domain comprises an scFv having the amino acid sequence of SEQ ID NO:55 or SEQ ID NO:56. In certain embodiments, the extracellular antigen binding domain comprises an scFv having the amino acid sequence of SEQ ID NO:59 or SEQ ID NO:60. In certain embodiments, the extracellular antigen binding domain comprises an scFv having the amino acid sequence of SEQ ID NO:63 or SEQ ID NO:64. In certain embodiments, the extracellular antigen binding domain comprises an scFv having the amino acid sequence of SEQ ID NO:67 or SEQ ID NO:68.

With respect to the transmembrane domain, in various embodiments, the CAR is designed to comprise a transmembrane domain that is fused to the extracellular domain of the CAR. In one embodiment, the transmembrane domain is one that naturally is associated with one of the domains in the CAR. In some instances, the transmembrane domain can be selected or modified by amino acid substitution to avoid binding of such domains to the transmembrane domains of the same or different surface membrane proteins to minimize interactions with other members of the receptor complex. In another embodiment, the transmembrane domain is capable of homodimerization with another CAR on the CAR T cell surface. In another embodiment, the amino acid sequence of the transmembrane domain may be modified or substituted so as to minimize interactions with the binding domains of the native binding partner present in the same CAR T cell.

The transmembrane domain may be derived from any naturally occurring membrane-bound or transmembrane protein. In one embodiment, the transmembrane region is capable of signaling to the intracellular domain(s) whenever the CAR has bound to a target. In some embodiments, the transmembrane domain comprises the transmembrane region(s) of one or more proteins selected from the group consisting of TCR α chain, TCR β chain, TCR ζ chain, CD28, CD38, CD45, CD4, CD5, CD8, CD9, CD16, CD22, DLL3, CD37, CD64, CD80, CD86, CD134, CD137, and CD154. In some embodiments, the transmembrane domain comprises the transmembrane region(s) of one or more proteins selected from the group consisting of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), SLAMF6 (NTB-A, Ly108), SLAM (SLAMFI, CD150, IPO-3), BLAME (SLAMF8), SELPLG (CD162), LTBR, PAG/Cbp, NKG2D, and NKG2C.

The extracellular DLL3-binding domain (e.g., DLL3-binding scFv domain) domain can be connected to the transmembrane domain by a hinge region. A variety of hinges can be employed, including but not limited to the human Ig (immunoglobulin) hinge (e.g., an IgG4 hinge, an IgD hinge), a Gly-Ser linker, a (G₄S)₄ linker, a KIR2DS2 hinge, and a CD8α hinge.

The intracellular signaling domain of the CAR of the invention is responsible for activation of at least one of the specialized functions of the immune cell (e.g., cytolytic activity or helper activity, including the secretion of cytokines, of a T cell) in which the CAR has been placed in. Thus, as used herein, the term “intracellular signaling domain” refers to the portion of a protein which transduces an effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain of the CAR comprises a primary signaling domain (i.e., a functional signaling domain derived from a stimulatory molecule) and one or more costimulatory signaling domains (i.e., functional signaling domains derived from at least one costimulatory molecule).

As used herein, the term “stimulatory molecule” refers to a molecule expressed by an immune cell, e.g., a T cell, an NK cell, or a B cell, that provide the cytoplasmic signaling sequence(s) that regulate activation of the immune cell in a stimulatory way for at least some aspect of the immune cell signaling pathway. In one embodiment, the signal is a primary signal that is initiated by, for instance, binding of a TCR/CD3 complex with an MHC molecule loaded with a peptide, and which leads to mediation of a T cell response, including, but not limited to, proliferation, activation, differentiation, and the like.

Primary signaling domains that act in a stimulatory manner may contain signaling motifs which are known as immunoreceptor tyrosine-based activation motifs or ITAMs. Examples of ITAM containing cytoplasmic signaling sequences that are of particular use in the invention include those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In one embodiment, the primary signaling domain in any one or more CARs of the invention comprises a cytoplasmic signaling sequence derived from CD3-zeta.

In some embodiments, the primary signaling domain is a functional signaling domain of TCR zeta, FcR gamma, FcR beta, CD3 gamma, CD3 delta, CD3 epsilon, CD5, CD22, CD79a, CD79b, CD66d, 4-1BB, and/or CD3-zeta. In an embodiment, the intracellular signaling domain comprises a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and/or DAP12. In a particular embodiment, the primary signaling domain is a functional signaling domain of the zeta chain associated with the T cell receptor complex.

As used herein, the term “costimulatory molecule” refers to a cognate binding partner on a T cell that specifically binds with a costimulatory ligand, thereby mediating a costimulatory response by the T cell, such as, but not limited to, proliferation. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1, CDIIa/CD18), CD2, CD7, CD258 (LIGHT), NKG2C, B7-H3, and a ligand that specifically binds with CD83, and the like. Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDIId, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, and a ligand that specifically binds with CD83. In some embodiments, the costimulatory signaling domain of the CAR is a functional signaling domain of a costimulatory molecule described herein, e.g., OX40, CD27, CD28, CD30, CD40, PD-1, CD2, CD7, CD258, NKG2C, B7-H3, a ligand that binds to CD83, ICAM-1, LFA-1 (CDIIa/CD18), ICOS and 4-1BB (CD137), or any combination thereof.

As used herein, the term “signaling domain” refers to the functional portion of a protein which acts by transmitting information within the cell to regulate cellular activity via defined signaling pathways by generating second messengers or functioning as effectors by responding to such messengers.

The cytoplasmic signaling sequences within the cytoplasmic signaling portion of the CAR of the invention may be linked to each other in a random or specified order. Optionally, a short oligo- or polypeptide linker, for example, between 2 and 10 amino acids in length may form the linkage.

Another aspect of the present invention provides a nucleic acid encoding a DLL3-targeting CAR disclosed herein. The nucleic acid is useful for expressing the CAR in an effector cell (e.g., T cell) by introducing the nucleic acid to the cell.

Modifications to the nucleic acid sequences may be made to create an equivalent or improved variant ofthe invention, for example, by changing one or more ofthe codons according to the codon degeneracy table. A DNA codon degeneracy table is provided in Table 10.

TABLE 10
Amino Acid Codons

| | One | Three | | | | | | |
|---|---|---|---|---|---|---|---|---|
| | letter | letter | | | | | | |
| Amino acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |
| Threonine | T | Thr | ACA | ACC | ACG | ACU | | |
| Valine | V | Val | GUA | GUC | GUG | GUU | | |
| Tryptophan | W | Trp | UGG | | | | | |
| Tyrosine | Y | Tyr | UAC | UAU | | | | |

In certain embodiments, the nucleic acid is a DNA molecule (e.g., a cDNA molecule). In certain embodiments, the nucleic acid further comprises an expression control sequence (e.g., promoter and/or enhancer) operably linked to the CAR coding sequence. In certain embodiments, the present invention provides a vector comprising the nucleic acid. The vector can be a viral vector (e.g., AAV vector, lentiviral vector, or adenoviral vector) or a non-viral vector (e.g., plasmid).

In certain embodiments, the nucleic acid is an RNA molecule (e.g., an mRNA molecule). A method for generating mRNA for use in transfection can involve in vitro transcription of a template with specially designed primers, followed by polyA addition, to produce an RNA construct containing 3′ and 5′ untranslated sequences, a 5′ cap and/or Internal Ribosome Entry Site (IRES), the nucleic acid to be expressed, and a polyA tail, typically 50-2000 bases in length. The RNA molecule can be further modified to increase translational efficiency and/or stability, e.g., as disclosed in U.S. Pat. Nos. 8,278,036; 8,883,506, and 8,716,465. RNA molecules so produced can efficiently transfect different kinds of cells.

In one embodiment, the nucleic acid encodes an amino acid sequence comprising a signal peptide at the amino-terminus of the CAR. Such signal peptide can facilitate the cell surface localization of the CAR when it is expressed in an effector cell, and is cleaved from the CAR during cellular processing. In one embodiment, the nucleic acid encodes an amino acid sequence comprising a signal peptide at the N-terminus of the extracellular DLL3-binding domain (e.g., DLL3-binding scFv domain).

RNA or DNA can be introduced into target cells using any of a number of different methods, for instance, commercially available methods which include, but are not limited to, electroporation, cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)).

Another aspect of the present invention provides an immune effector cell expressing the DLL3-targeting CAR. Also provided is an immune effector cell comprising the nucleic acid encoding the DLL3-targeting CAR. The immune effector cells include but are not limited to T cells and NK cells. In certain embodiments, the T cell is selected from a CD8⁺ T cell, a CD4⁺ T cell, and an NKT cell. The T cell or NK cell can be a primary cell or a cell line.

The immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors, by methods known in the art. The immune effector cells can also be differentiated in vitro from a pluripotent or multipotent cell (e.g., a hematopoietic stem cell). In some embodiments, the present invention provides a pluripotent or multipotent cell (e.g., a hematopoietic stem cell) expressing the DLL3-targeting CAR or comprising a nucleic acid disclosed herein.

In certain embodiments, the immune effector cells are isolated and/or purified. For example, regulatory T cells can be removed from a T cell population using a CD25-binding ligand. Effector cells expressing a checkpoint protein (e.g., PD-1, LAG-3, or TIM-3) can be removed by similar methods. In certain embodiments, the effector cells are isolated by a positive selection step. For example, a population of T cells can be isolated by incubation with anti-CD3/anti-CD28-conjugated beads. Other cell surface markers, such as IFN-7, TNF-α, IL-17A, IL-2, IL-3, IL-4, GM-CSF, IL-10, IL-13, granzyme B, and perforin, can also be used for positive selection.

Immune effector cells may be activated and expanded generally using methods known in the art, e.g., as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publications Nos. 2006/0121005 and 2016/0340406. For example, in certain embodiments, T cells can be expanded and/or activated by contact with an anti-CD3 antibody and an anti-CD28 antibody, under conditions appropriate for stimulating proliferation of the T cells. The cells can be expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. Multiple cycles of stimulation may be desirable for prolonged cell culture (e.g., culture for a period of 60 days or more). In certain embodiments, the cell culture comprises serum (e.g., fetal bovine or human serum), interleukin-2 (IL-2), insulin, IFN-γ, IL-4, IL-7, GM-CSF, IL-10, IL-12, IL-15, TGFβ, TNF-α, or a combination thereof. Other additives for the growth of cells known to the skilled person, e.g., surfactant, plasmanate, and reducing agents such as N-acetyl-cysteine and 2-mercaptoethanol, can also be included in the cell culture. In certain embodiments, the immune effector cell of the present invention is a cell obtained from in vitro expansion.

### DLL3/CD3-Directed Bispecific T-Cell Engagers

In certain embodiments, the present invention provides a DLL3/CD3-directed bispecific T-cell engager comprising an antigen-binding site that binds DLL3 disclosed herein. In certain embodiments, the DLL3/CD3-directed bispecific T-cell engager comprises an amino acid sequence at least 90% (e.g., 90%, 91%, 92%, 93%, 94%, 95%, 96%, 97%, 98%, 99%, or 100%) identical to SEQ ID NO:33. In certain embodiments, the DLL3/CD3-directed bispecific T-cell engager comprises an amino acid sequence at least 90% (e.g., 90%, 91%, 92%, 93%, 94%, 95%, 96%, 97%, 98%, 99%, or 100%) identical to SEQ ID NO:34. In certain embodiments, the cytokine is connected to the Fc domain directly or via a linker.

In certain embodiments, the DLL3/CD3-directed bispecific T-cell engager further comprises an antigen-binding site that binds CD3. Exemplary antigen-binding sites that bind CD3 are disclosed in International Patent Application Publication Nos. WO2014/051433 and WO2017/097723.

Another aspect of the present invention provides a nucleic acid encoding at least one polypeptide of the DLL3/CD3-directed bispecific T-cell engager, wherein the polypeptide comprises an antigen binding site that binds DLL3. In certain embodiments, the nucleic acid further comprises a nucleotide sequence encoding a signal peptide that, when expressed, is at the N-terminus of one or more of the polypeptides of the DLL3/CD3-directed bispecific T-cell engager. Also provided is a vector (e.g., a viral vector) comprising the nucleic acid, a producer cell comprising the nucleic acid or vector, and a producer cell expressing the DLL3/CD3-directed bispecific T-cell engager.

### Immunocytokines

In certain embodiments, the present invention provides an immunocytokine comprising an antigen-binding site that binds DLL3 disclosed herein and a cytokine. Any cytokine (e.g., pro-inflammatory cytokines) known in the art can be used, including but not limited to IL-2, IL-4, IL-10, IL-12, IL-15, TNF, IFNα, IFNγ, and GM-CSF. More exemplary cytokines are disclosed in U.S. Pat. No. 9,567,399. In certain embodiments, the antigen-binding site is connected to the cytokine by chemical conjugation (e.g., covalent or noncovalent chemical conjugation). In certain embodiments, the antigen-binding site is connected to the cytokine by fusion of polypeptide. The immunocytokine can further comprise an Fc domain connected to the antigen-binding site that binds DLL3. In certain embodiments, the immunocytokine comprises an amino acid sequence at least 90% (e.g., 90%, 91%, 92%, 93%, 94%, 95%, 96%, 97%, 98%, 99%, or 100%) identical to SEQ ID NO:33. In certain embodiments, the immunocytokine comprises an amino acid sequence at least 90% (e.g., 90%, 91%, 92%, 93%, 94%, 95%, 96%, 97%, 98%, 99%, or 100%) identical to SEQ ID NO:34. In certain embodiments, the cytokine is connected to the Fc domain directly or via a linker.

Another aspect of the present invention provides a nucleic acid encoding at least one polypeptide of the immunocytokine, wherein the polypeptide comprises an antigen binding site that binds DLL3. In certain embodiments, the nucleic acid further comprises a nucleotide sequence encoding a signal peptide that, when expressed, is at the N-terminus of one or more of the polypeptides of the immunocytokine. Also provided is a vector (e.g., a viral vector) comprising the nucleic acid, a producer cell comprising the nucleic acid or vector, and a producer cell expressing the immunocytokine.

### Antibody-Drug Conjugates

In certain embodiments, the present invention provides an antibody-drug conjugate comprising an antigen-binding site that binds DLL3 disclosed herein and a cytotoxic drug moiety. Exemplary cytotoxic drug moieties are disclosed in International Patent Application Publication Nos. WO2014/160160 and WO2015/143382. In certain embodiments, the cytotoxic drug moiety is selected from auristatin, N-acetyl-γ calicheamicin, maytansinoid, pyrrolobenzodiazepine, and SN-38. The antigen-binding site can be connected to the cytotoxic drug moiety by chemical conjugation (e.g., covalent or noncovalent chemical conjugation). In certain embodiments, the antibody-drug conjugate further comprises an Fc domain connected to the antigen-binding site that binds DLL3. In certain embodiments, the antibody-drug conjugate comprises an amino acid sequence at least 90% (e.g., 90%, 91%, 92%, 93%, 94%, 95%, 96%, 97%, 98%, 99%, or 100%) identical to SEQ ID NO:33. In certain embodiments, the antibody-drug conjugate comprises an amino acid sequence at least 90% (e.g., 90%, 91%, 92%, 93%, 94%, 95%, 96%, 97%, 98%, 99%, or 100%) identical to SEQ ID NO:34. In certain embodiments, the cytotoxic drug moiety is connected to the Fc domain directly or via a linker.

### Immunotoxins

In certain embodiments, the present invention provides an immunotoxin comprising an antigen-binding site that binds DLL3 disclosed herein and a cytotoxic peptide moiety. Any cytotoxic peptide moiety known in the art can be used, including but not limited to ricin, Diphtheria toxin, and Pseudomonas exotoxin A. More exemplary cytotoxic peptides are disclosed in International Patent Application Publication Nos. WO2012/154530 and WO2014/164680. In certain embodiments, the cytotoxic peptide moiety is connected to the protein by chemical conjugation (e.g., covalent or noncovalent chemical conjugation). In certain embodiments, the cytotoxic peptide moiety is connected to the protein by fusion of polypeptide. The immunotoxin can further comprise an Fc domain connected to the antigen-binding site that binds DLL3. In certain embodiments, the immunotoxin comprises an amino acid sequence at least 90% (e.g., 90%, 91%, 92%, 93%, 94%, 95%, 96%, 97%, 98%, 99%, or 100%) identical to SEQ ID NO:33. In certain embodiments, the immunotoxin comprises an amino acid sequence at least 90% (e.g., 90%, 91%, 92%, 93%, 94%, 95%, 96%, 97%, 98%, 99%, or 100%) identical to SEQ ID NO:34. In certain embodiments, the cytotoxic peptide moiety is connected to the Fc domain directly or via a linker.

Another aspect of the present invention provides a nucleic acid encoding at least one polypeptide of the immunotoxin, wherein the polypeptide comprises an antigen binding site that binds DLL3. In certain embodiments, the nucleic acid further comprises a nucleotide sequence encoding a signal peptide that, when expressed, is at the N-terminus of one or more of the polypeptides of the immunotoxin. Also provided is a vector (e.g., a viral vector) comprising the nucleic acid, a producer cell comprising the nucleic acid or vector, and a producer cell expressing the immunotoxin.

### III. Therapeutic Compositions and Their Use

The invention provides methods for treating cancer using a multi-specific binding protein described herein and/or a pharmaceutical composition described herein. The methods may be used to treat a variety of cancers which express DLL3 by administering to a patient in need thereof a therapeutically effective amount of a multi-specific binding protein described herein.

The therapeutic method can be characterized according to the cancer to be treated. for example, in certain embodiments, the cancer is small cell lung cancer, large cell neuroendocrine carcinoma, glioblastoma, ewing's sarcoma, or cancers with neuroendocrine phenotype.

For example, in certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cycstic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The cancer to be treated can be characterized according to the presence of a particular antigen expressed on the surface of the cancer cell. In certain embodiments, the cancer cell can express one or more of the following in addition to DLL3: CD2, CD19, CD20, CD30, CD38, CD40, CD52, CD70, EGFR/ERBB1, IGF1R, HER3/ERBB3, HER4/ERBB4, MUC1, TROP2, cMET, SLAMF7, PSCA, MICA, MICB, TRAILR1, TRAILR2, MAGE-A3, B7.1, B7.2, CTLA4, and PD1.

### IV. Combination Therapy

Another aspect of the invention provides for combination therapy. Multi-specific binding proteins described herein be used in combination with additional therapeutic agents to treat the cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDCl₇ Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the invention can also be used as an adjunct to surgical removal of the primary lesion.

The amount of multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

### V. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In one aspect, the present disclosure provides a formulation of a protein, which includes a DLL3-binding site described herein, and a pharmaceutically acceptable carrier.

In some embodiments, the pharmaceutical composition includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:33, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:34. In some embodiments, the pharmaceutical composition includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:35, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:34. In some embodiments, the pharmaceutical composition includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:36, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:34. In some embodiments, the pharmaceutical composition includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:37, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:34. In some embodiments, the pharmaceutical composition includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:38, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:34. In some embodiments, the pharmaceutical composition includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:1, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:2. In some embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:3, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:4. In some embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:5, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:6. In some embodiments, the formulation includes a protein that includes an antigen-binding site with a heavy chain variable domain having an amino acid sequence at least 90% (e.g., 91%, 92%, 93%, 94%, 95%, 96%, 97%, 98%, 99%, or 100%) identical to the amino acid sequence of SEQ ID NO:7, and a light chain variable domain having an amino acid sequence at least 90% (e.g., 91%, 92%, 93%, 94%, 95%, 96%, 97%, 98%, 99%, or 100%) identical to the amino acid sequence of SEQ ID NO:8.

The composition can be formulated for use in a variety of drug delivery systems. One or more physiologically acceptable excipients or carriers can be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

For example, this present disclosure could exist in an aqueous pharmaceutical formulation including a therapeutically effective amount of the protein in a buffered solution forming a formulation. Aqueous carriers can include sterile water for injection (SWFI), bacteriostatic water for injection (BWFI), a pH buffered solution (e.g. phosphate-buffered saline), sterile saline solution, Ringer's solution or dextrose solution. In certain embodiments, an aqueous formulation is prepared including the protein disclosed herein in a pH-buffered solution. The pH of the preparations typically will be between 3 and 11, more preferably between 5 and 9 or between 6 and 8, and most preferably between 7 and 8, such as 7 to 7.5. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g. 1.53 mg/ml), about 0.9 mg/ml of sodium dihydrogen phosphate dihydrate (e.g., 0.86), and about 6.2 mg/ml of sodium chloride (e.g., 6.165 mg/ml). In certain embodiments, the buffer system includes 1-1.5 mg/ml of citric acid, 0.25 to 0.5 mg/ml of sodium citrate, 1.25 to 1.75 mg/ml of disodium phosphate dihydrate, 0.7 to 1.1 mg/ml of sodium dihydrogen phosphate dihydrate, and 6.0 to 6.4 mg/ml of sodium chloride. The pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In some embodiments, the formulation include an aqueous carrier, which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/ml. In certain embodiments, the concentration of mannitol may be about 7.5 to 15 mg/ml. In certain embodiments, the concentration of mannitol may be about 10-14 mg/ml. In certain embodiments, the concentration of mannitol may be about 12 mg/ml. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative, which is added to the formulations herein to reduce bacterial action. The addition of a preservative may, for example, facilitate the production of a multi-use (multiple-dose) formulation.

In some embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

Deamidation is a common product variant of peptides and proteins that may occur during fermentation, harvest/cell clarification, purification, drug substance/drug product storage and during sample analysis. Deamidation is the loss of NH3 from a protein forming a succinimide intermediate that can undergo hydrolysis. The succinimide intermediate results in a 17 u mass decrease of the parent peptide. The subsequent hydrolysis results in an 18 u mass increase. Isolation of the succinimide intermediate is difficult due to instability under aqueous conditions. As such, deamidation is typically detectable as 1 u mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation. In certain embodiments, the liquid formulation of the present disclosure may be preserved under conditions of pH and humidity to prevent deamination of the protein product.

In some embodiment, the formulation is a lyophilized formulation. In certain embodiments, the formulation is freeze-dried (lyophilized) and contained in about 12-60 vials. In certain embodiments, the formulation is freeze-dried and 45 mg of the freeze-dried formulation may be contained in one vial. In certain embodiments, the about 40 mg-about 100 mg of freeze-dried formulation is contained in one vial. In certain embodiments, freeze dried formulation from 12, 27, or 45 vials are combined to obtained a therapeutic dose of the protein in the intravenous drug formulation. The formulation may be a liquid formulation. In some embodiments, a liquid formulation is stored as about 250 mg/vial to about 1000 mg/vial. In certain embodiments, the liquid formulation is stored as about 600 mg/vial. In certain embodiments, the liquid formulation is stored as about 250 mg/vial.

In some embodiments, the lyophilized formulation includes the proteins described herein and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative. The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

In certain embodiments, the lyophilized protein product is constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution. In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution. In certain embodiments, the lyophilized protein product of the instant disclosure is constituted to about 4.5 mL water for injection and diluted with 0.9% saline solution (sodium chloride solution).

The protein compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The resulting compositions in solid form may be packaged in multiple single dose units, each containing a fixed amount of the above-mentioned agent or agents. The composition in solid form can also be packaged in a container for a flexible quantity.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica. Chimica. Acta. 308: 43-53, 2001; Steimer et al., Clinica. Chimica. Acta. 308: 33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight. Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the invention. The patent application specifically contemplates all combinations and permutations of the aspects and embodiments.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following examples are merely illustrative and are not intended to limit the scope or content of the invention in any way.

### Example 1. Binding Kinetics of Anti-DLL3 Antibodies to Different Variants of DLL3

ECD of human DLL3 was purchased from Adipogen (AG-40B-0151 has a FLAG-tag) and further purified using size exclusion chromatography. Recombinant His-tagged proteins of different domains of human DLL3 (N-terminal, EGF2-6, EGF2-6, EGF4-6, EGF5-6) were expressed in a cell line and purified using size exclusion chromatography.

The binding kinetics of different anti-DLL3 antibodies to recombinant proteins of different domains of human DLL3 were studied by Surface Plasmon Resonance using a Biacore 8K instrument. These anti-DLL3 antibodies were produced from mouse hybridomas, and each included a heavy chain variable region and light chain variable region described herein. Briefly, antibodies recognizing human IgG Fc and antibodies recognizing mouse IgG Fc were immobilized on different channels of a Biacore 8K chip to allow simultaneous analysis of human and murine anti-DLL3 antibodies. Murine anti-DLL3 antibodies were captured on the anti-mouse Fc channel of the Biacore chip. The human anti-DLL3 antibody from Stemcentrix was used as a control and was captured onto anti-human Fc channel of the Biacore chip. Different concentrations of DLL3 ECD, DLL3 N-terminal domain, EGF2-6, EGF3-6, EGF4-6, or EGF5-6 domains of DLL3 were injected. Experiments were performed at 37° C. Biacore 8K evaluation software was used for all data analysis. To obtain kinetic rate constants double-referenced data were fit to a 1:1 interaction model using Biacore 8K Evaluation software (GE Healthcare). The equilibrium binding constant K_{D} was determined by the ratio of binding rate constants k_{d}/kₐ.

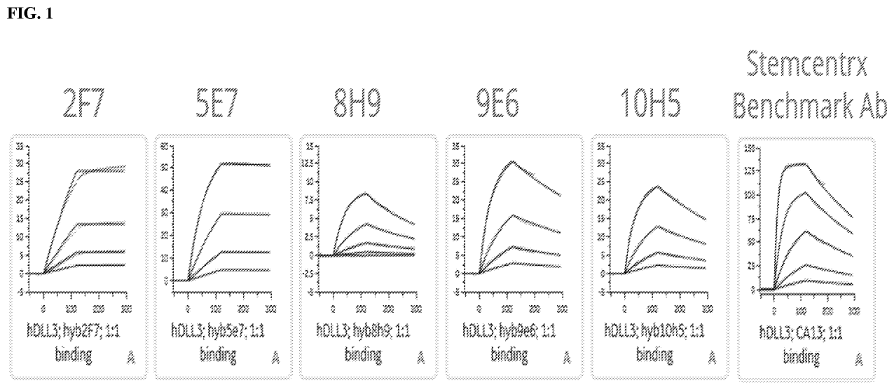

FIG. 1 shows binding profiles of murine anti-DLL3 antibodies to the ECD of human DLL3 (from Adipogen), obtained by SPR analysis at 37° C. Table 9 lists the calculated binding kinetics (K_{D}). The antibodies demonstrate a range of binding affinities to the DLL3 ECD from <0.011 to 8.44 nM. Stemcenrtx anti-DLL3 antibody was used as a control.

TABLE 9

| Antibody | Construct | kₐ (1/Ms) | k_{d} (1/s) | K_{D} (nM) |
|---|---|---|---|---|
| 2F7 | hDLL3 ECD | 7.21e+4 | ~1.15e−6 | <0.011* |
| 5E7 | hDLL3 ECD | 3.19e+5 | 6.47e−5 | 0.203 |
| 8H9 | hDLL3 ECD | 5.42e+5 | 4.58e−3 | 8.44 |
| 9E6 | hDLL3 ECD | 4.08e+5 | 2.08e−3 | 5.11 |
| 10H5 | hDLL3 ECD | 4.46e+5 | 2.74e−3 | 6.1 |
| Stemcentrx | hDLL3 ECD | 1.64e+6 | 3.14e−3 | 1.92 |
| Benchmark Ab | | | | |

*Kinetic data for 2F7 are approximate - off rate beyond the instrument sensitivity

### Mapping of Antibody Binding Epitope on DLL3

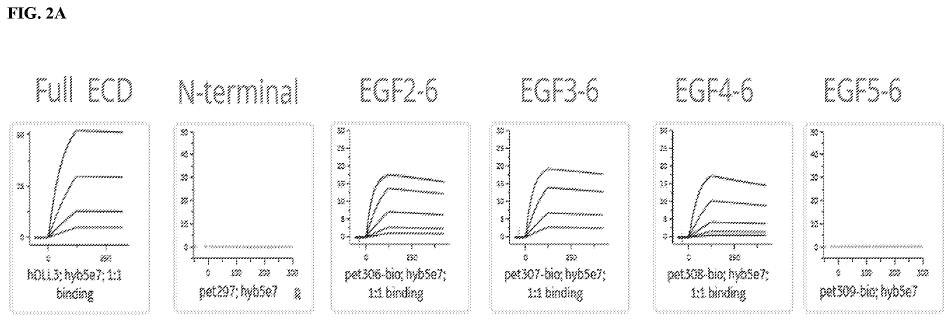

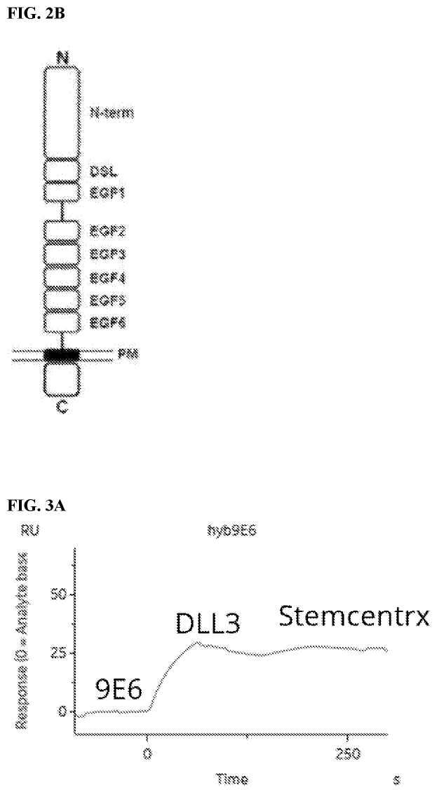

The binding epitope on DLL3 by the anti-DLL3 antibody which included a heavy chain variable region and light chain variable region of clone 5E7 (see Table 1) was obtained by SPR analysis at 37° C. FIG. 2A shows the binding kinetics of the anti-DLL3 antibody to different DLL3 domains (His-tagged N-terminus, EGF2-6, EGF3-6, EGF4-6, or EGF5-6) illustrated in FIG. 2B. The antibody binds to constructs incorporating EGF2-6, EGF3-6, and EGF4-6, but shows no binding to the N-terminus or to the EGF5-6 domain of DLL3, indicating that EGF4 is involved in binding to 5E7. Binding kinetics (K_{D}) of the antibody to different DLL3 domains are listed in Table 10.

TABLE 10

| Antibody | Construct | Kₐ (1/Ms) | K_{d} (1/s) | K_{D} (nM) |
|---|---|---|---|---|
| 5E7 | hDLL3 ECD | 3.19e+05 | 6.47e−05 | 0.203 |
| 5E7 | N-terminal | | No binding | |
| 5E7 | EGF2-6 | 5.7e+0.5 | 3.82e−04 | 0.669 |
| 5E7 | EGF3-6 | 1.45e+06 | 2.64e−04 | 0.184 |
| 5E7 | EGF4-6 | 5.46e+05 | 6.10e−04 | 1.12 |
| 5E7 | EGF5-6 | | No binding | |

### Example 2: Epitope Binning of Anti-DLL3 Antibodies

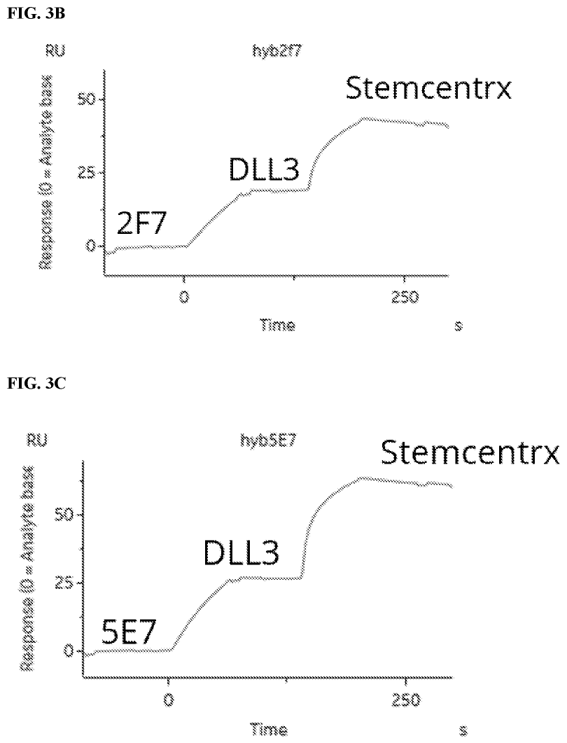

Binning of different anti-DLL3 antibodies against Stemcentrx antibody was performed by Surface Plasmon Resonance (SPR) using a Biacore 8K instrument. Briefly, murine anti-DLL3 antibodies were captured using an anti-mouse Fc antibody immobilized on a CM5 chip. This was followed by injections of human DLL3 ECD and the Stemcentrx anti-DLL3 antibody consecutively. Experiments were performed at 25° C. Biacore 8K evaluation software was used for all data analysis. FIGS. 3A-3C show the binning profiles of anti-DLL3 antibodies corresponding to 2F7 (FIG. 3B), 5E7 (FIG. 3C), and 9E6 (FIG. 3A) clones to the ECD of DLL3. The antibody including the 9E6 clone binds to an epitope on DLL3 overlapping the epitope to which the Stemcentrx anti-DLL3 antibody binds (FIG. 3A). The antibody including the 2F7 or 5E7 clone did not block the binding of the Stemcentrx antibody to the DLL3 ECD, suggesting that the antibodies corresponding to 2F7 (FIG. 3B) and 5E7 (FIG. 3C) bind to epitopes on DLL3 different from the one bound by the Stemcentrx antibody.

### Example 3: Determination of the Melting Temperatures of Anti-DLL3 Antibodies

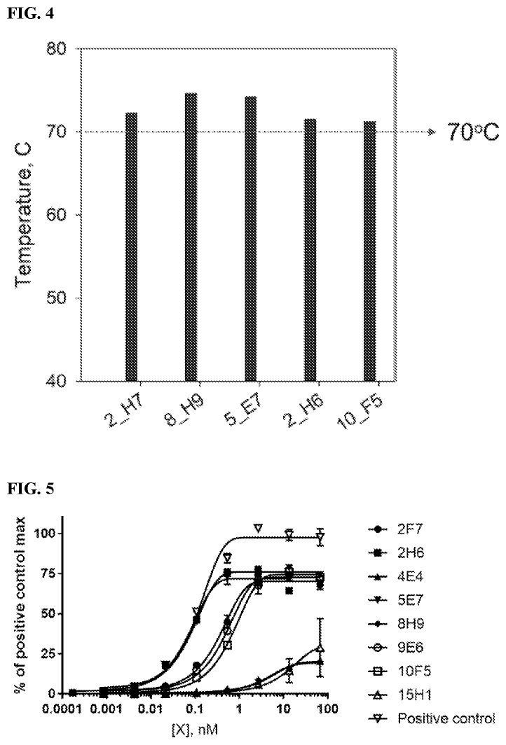

Determination of the melting temperature was done by a differential scanning fluorimetry analysis using New Applied Biosystems QuantStudio3 instrument from Thermo Fisher in the temperature range of 15-95° C. All samples were run in duplicate. Results were analyzed using Applied Biosystems Protein Thermal Shift™ Software Version 13. As shown in FIG. 4, anti-DLL3 antibodies corresponding to clones 2H7, 8H9, 5E7, 2H6, and 10F5 showed melting temperatures above 70° C.

### Example 4: Assessment of Binding to Recombinant Human DLL3 and Cross-Reactivity with DLL1/DLL4

To investigate the binding of anti-DLL3 antibodies to human DLL3, wells of high binding flat-bottom plates were coated with recombinant human DLL3 diluted to 0.5 μg/ml. To assess cross-reactivity of the antibodies to DLL1/DLL4, which are closely related family members to DLL3, plates were coated with recombinant human DLL1 diluted to 1 μg/ml, or human DLL4 diluted to 0.5 μg/ml. After blocking the plates with PBS containing 1% BSA, a test anti-DLL3 antibody and a positive control antibody for each of DLL3 (R&D Systems—MAB4315), DLL1 (BioLegend—MHD1-314) and DLL4 (Biolegend—MHD4-46) respectively were diluted serially starting from 10 μg/ml and added to the wells. Binding was detected using anti-mouse IgG-HRP and 3,3′,5,5′-Tetramethylbenzidine (TMB) substrate. Signals were normalized to the corresponding positive control antibody.

As shown in FIG. 5, robust binding of anti-DLL3 antibodies to DLL3 in a dose-dependent manner was observed. Table 11 lists the calculated EC₅₀ based on the binding data from FIG. 5. Asterisks indicate anti-DLL3 antibodies that did not reach saturation of binding to DLL3; the corresponding EC₅₀ values are therefore estimates.

TABLE 11

| DLL3 clone | ELISA EC₅₀ (nM) |
|---|---|
| 2F7 | 0.30 |
| 2H6 | 0.06 |
| 4E4 | 4.93* |
| 5E7 | 0.07 |
| 8H9 | 4.58* |
| 9E6 | 0.45 |
| 10F5 | 0.64 |
| 15H1 | 76.1* |
| Positive** | 0.10 |

*Does not reach saturation; value is an estimate.
**Commercial reagent mAb

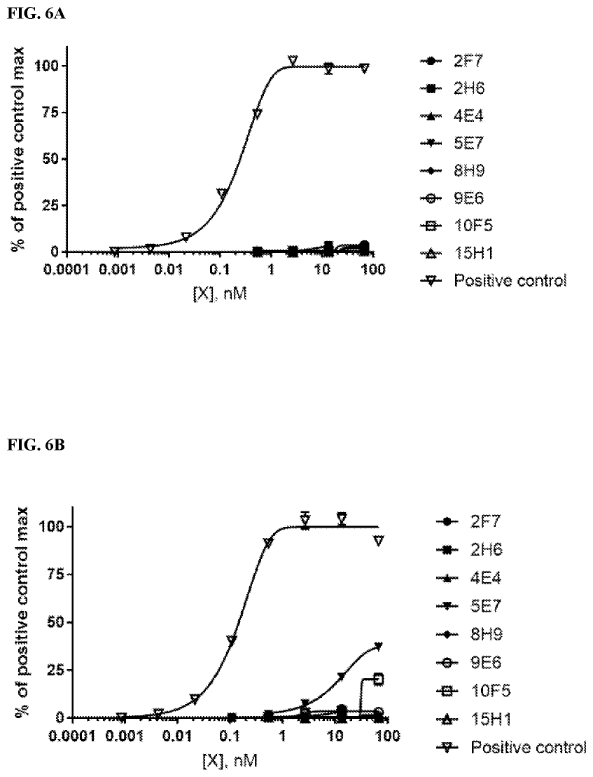

As shown in FIG. 6A, the anti-DLL3 antibodies displayed little cross-reactivity with human DLL1. As shown in FIG. 6B, the anti-DLL3 antibodies corresponding to clones 2F7, 2H6, 4E4, 8H9, 9E6, and 15H1 also displayed little cross-reactivity with human DLL4. Two antibodies corresponding to clones 5E7 and 10F5 showed weak cross-reactive binding to DLL4 at very high concentrations measured by ELISA. Binding signals were normalized to the corresponding positive control anti-DLL3 antibody and the positive control anti-DLL4 antibody.

### Example 5: Assessment of Antibody Binding to Cell-Expressed Human DLL3

Human small cell lung cancer line NCI-H82 that expresses DLL3 was used to assess the binding of the anti-DLL3 antibodies to DLL3. Antibodies were serially diluted starting from 2 μg/mL and then incubated with the cells. Binding was detected using a fluorophore-conjugated anti-mouse IgG secondary antibody. Cells were analyzed by flow cytometry, and binding was expressed as mean fluorescence intensity (MFI) relative to the signal from secondary antibody only control.

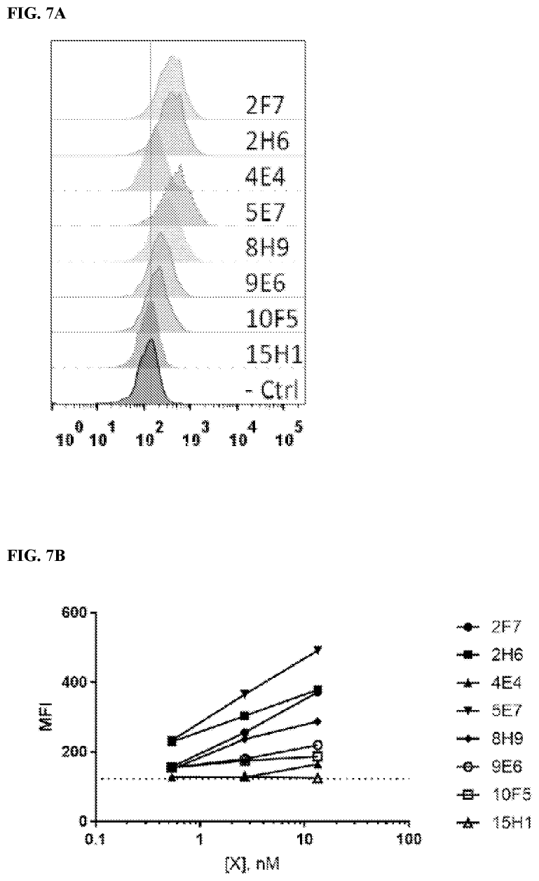

As shown in FIG. 7A, the anti-DLL3 antibodies corresponding to clones 2F7, 2H6, 4E4, 5E7, 8H9, 9E6, 10F5, and 15H1 (2 μg/mL) bound to DLL3 expressed on NCI-H82 cells. And as shown in FIG. 7B, the binding of the anti-DLL3 antibodies corresponding to clones 5E7, 2H6, 2F7, and 8H9 to DLL3 on NCI-H82 cells was dose-dependent, while the binding of the antibodies corresponding to clones 9E6 and 10F5 to DLL3 was dose-dependent above a 5 nM dose, and the binding of the antibody corresponding to clone 15H1 to DLL3 on NCI-H82 cells was independent of dose.

### Example 6: Determination of Extent of Antibody Internalization by DLL3⁺ SCLC Lines

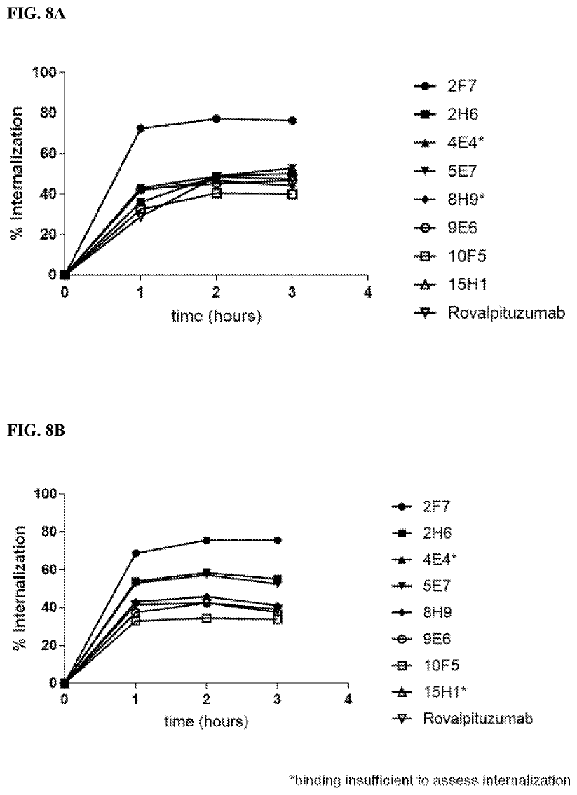

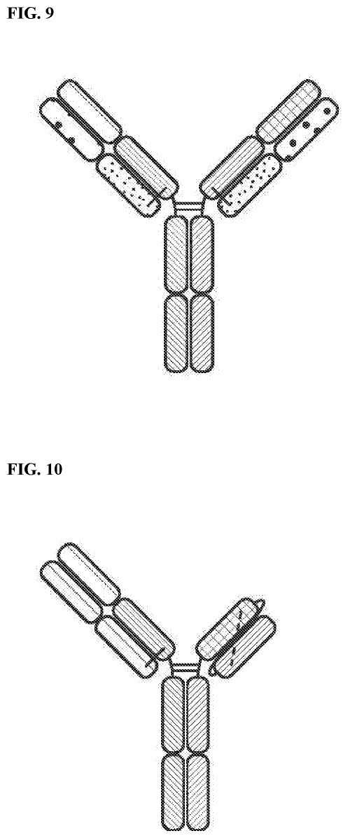

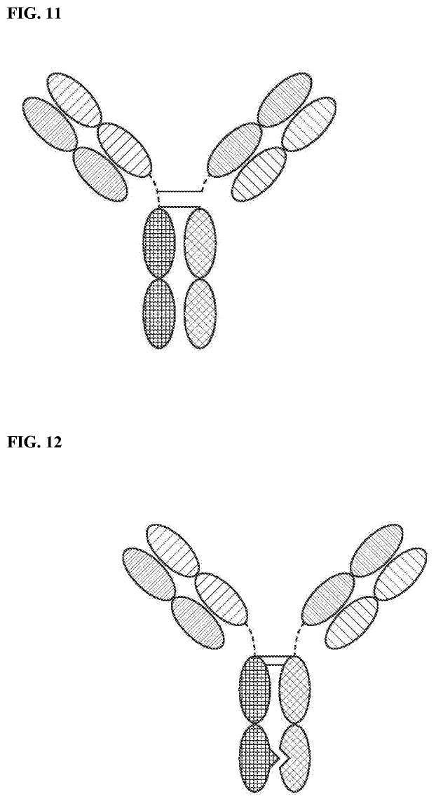

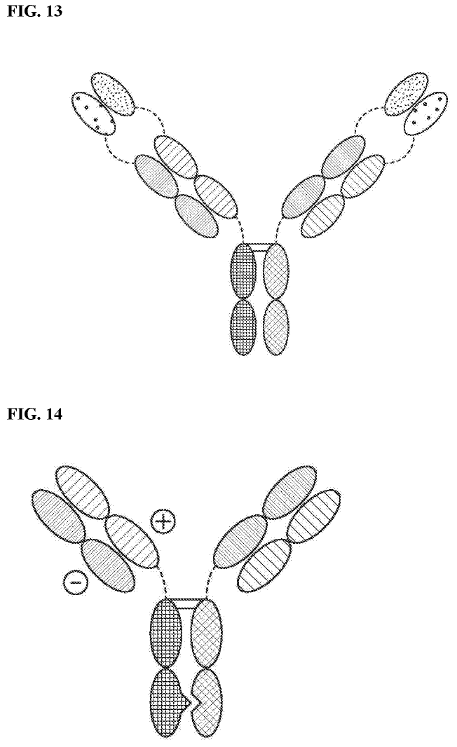

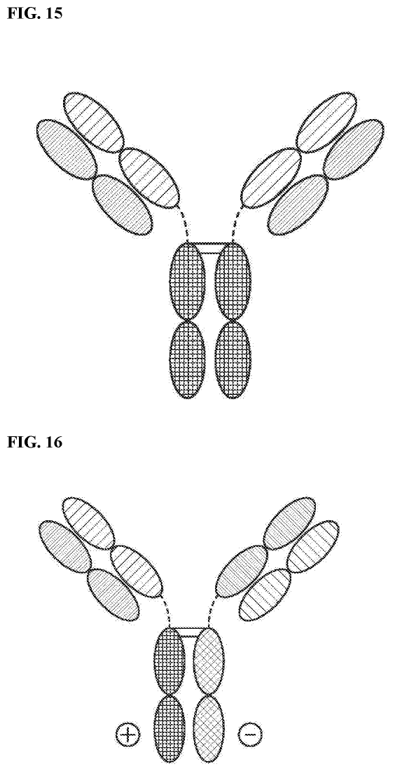

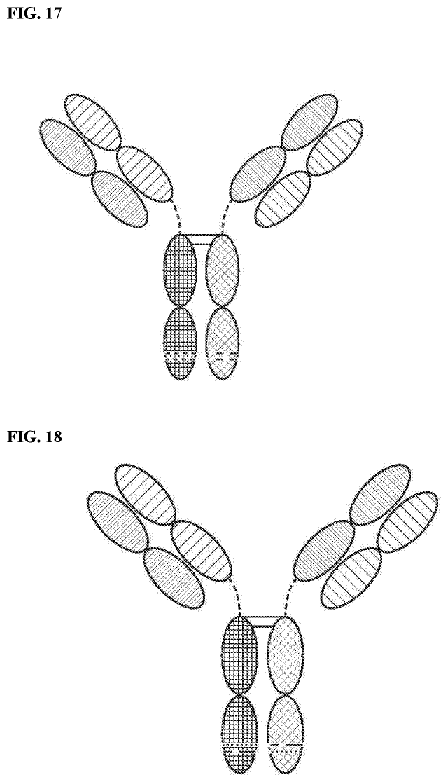

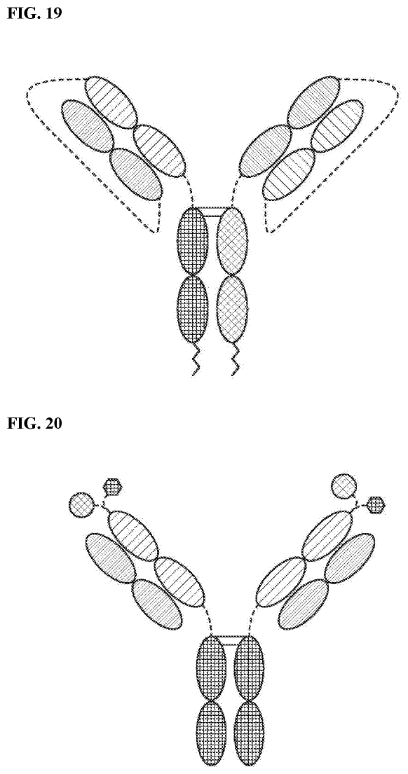

Human small cell lung cancer cell lines SHP-77 and DMS-79 expressing DLL3 were used to assess internalization of the anti-DLL3 antibodies upon binding to DLL3 on the surface of the cells. The antibodies were diluted to 10 μg/mL and incubated with the cells at 37° C. for 1, 2 or 3 hours or on ice for 20 minutes. The remaining surface-bound antibodies were then detected using a fluorophore-conjugated anti-mouse IgG secondary antibody. Cells were analyzed by flow cytometry, and the antibody internalization was calculated as a percentage loss of mean fluorescence intensity (MFI) in comparison with the corresponding control condition, when the cells were incubated with the antibody on ice. As shown in FIGS. 8A-8B, significant internalization of the anti-DLL3 antibodies on SHP-77 cells (FIG. 8A) and DMS-79 cells (FIG. 8B) was observed.

### Example 7: Humanization of Murine Antibody 5E7

Humanization of mouse 5E7 was accomplished by grafting mouse CDRs to appropriate human frameworks using molecular operating environment (MOE) protein modelling software. The CDR grafting was based on combination of best sequence match to human frameworks and by homology model. Human germline VH1-3 was selected as the most appropriate acceptor framework for variable heavy chain. For maintaining binding and structural integrity of the VH domain, three residues of the selected human framework were mutated back to the original mouse framework residues. Human germline VK1-39 was selected as the most appropriate acceptor framework for variable light chain. For maintaining binding and structural integrity of the VL domain, three residues of the selected human framework were mutated back to the original mouse frame work residues. From this effort the best variant (clone h5E7) was selected.

The three residues in the VH mutated back to the original mouse framework residues were at Kabat positions 44, 71, and 76, as bolded and underlined in the h5E7 VH sequence below. The heavy chain CDR sequences were also identified and are underlined below.

h5E7 VH sequence
[SEQ ID NO: 33]
QVQLVQSGAEVKKPGASVKVSCKASGFNIKDDYIHWVRQAPGQGLEWMG
WIDSENGDTEYASKFQGRVTITADTSANTAYMELSSLRSEDTAVYYCAT
SSYYSYDLFVYWGQGTLVTVSS

The three residues in the VL mutated back to the original mouse framework residues were at Kabat positions 2, 36, and 42, as bolded and underlined in the h5E7 VL sequence below. The light chain CDR sequences were also identified and are underlined below.

h5E7 VL sequence
[SEQ ID NO: 34]
DVQMTQSPSSLSASVGDRVTITCKSSQSLLHSNGKTYLNWLQQKPGQAP
KLLLYLVSKLESGVPSRFSGSGSGTDYTLTISSLQPEDFATYYCLQTTH
LYTFGQGTKLEIK

### Example 8: Affinity Maturation of h5E7 by CDRH3 Mutation

A CDRH3 focused library with single, double and triple mutants of h5E7 was displayed as single chain variable fragment (scFv) on the surface of Saccharomyces cerevisiae. The starting library diversity was estimated to be around 10⁶. Three rounds of selections were carried out. The first round of selection was performed by magnetic activated cell sorting (MACS) and enriched clones that bound to 100 nM human DLL3 ECD (ECD of DLL3 was purchased from Adipogen (AG-40B-0151) and further purified in house using size exclusion chromatography before use in this experiment). The second and third rounds of selection were carried out on a fluorescence activated cell sorter (FACS). During the second round of selection, biotinylated human DLL3 was titrated down to 1 nM and variants in the library that bound better than parent h5E7 were gated and collected. The third round of selection was focused on enriching binders that have slower off-rate (kd) than the parent h5E7 clone. This was achieved by competing off bound biotinylated human DLL3 from relatively faster kd variants with excess of unbiotinylated hDLL3 or with the murine 5E7 monoclonal antibody (mAb). The clones enriched from the second and third rounds included h5E7-YD-C6, h5E7-YD-F3, h5E7-YD-A6, and h5E7-YD-B5, the sequence of which are showed in Table 1. Consensus sequences of the humanized 5E7 variants are also provided in Table 1.

The murine 5E7 and all the humanized versions were cloned and expressed as IgG1 mAbs. All heavy chain variable regions (including mouse 5E7) were cloned into the N-terminus of human IgG1 CH1-CH2-CH3 constant region. All light chain variable regions (including mouse 5E7) were cloned into N-terminus of human constant Kappa region. All clones were expressed in the EXPI293 system and purified using protein A MabSelect SuRe resin. When necessary, an additional step of SEC purification was performed.

The kinetics of binding of different humanized variants and parental murine 5E7 antibody to human DLL3-His was studied by Surface Plasmon Resonance using a Biacore 8K instrument. Briefly, anti-hFc and anti-mFc IgG proteins were covalently immobilized onto different channels of CM5 chip to allow simultaneous analysis of human and murine antibodies. m5E7 was captured on the anti-mouse Fc channel and humanized variants were captured on the anti-human Fc channel at a desired capture level of ˜56 RU in HBS-EP+ buffer supplemented with 0.1% BSA. Three buffer blanks and human DLL3-His (analyte) at concentrations 0.411-300 nM (in three fold dilutions) were injected over the surface with captured murine or humanized 5E7 for 300 second association time and let dissociate for 600 second at a flow rate of 30 μL/min. The surfaces were subjected to regeneration with three 20-second pulses of 10 mM glycine pH 1.70 at a flow rate of 100 μL/min between every concentration of analyte. The experiment was conducted at 37° C. Data were double referenced and a 1:1 fit model was applied to the sensorgrams in the Biacore Insight Evaluation software.

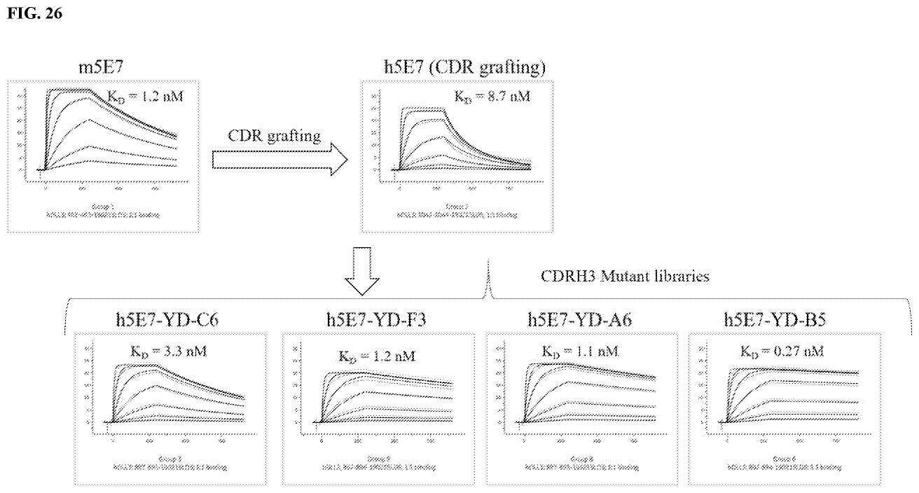

As shown in FIG. 26, the murine 5E7 and the humanized variants all bound human DLL3-His. The kinetic parameters of human DLL3-His binding to murine and humanized variants of 5E7 antibody variants were calculated and shown in Table 12.

TABLE 12

| Antibody | | | | KD | |
|---|---|---|---|---|---|
| clone | Antigen | kₐ (1/Ms) | k_{d} (1/s) | (nM) | CDRH3 |
| 5E7 | hDLL3 | 1.19 × 106 | 1.45 × 10⁻³ | 1.22 | SSYYSY |
| | | | | | DLFVY |
| h5E7 | hDLL3 | 7.99 × 105 | 6.97 × 10⁻³ | 8.72 | SSYYSY |
| | | | | | DLFVY |
| h5E7-YD-C6 | hDLL3 | 4.09 × 105 | 1.37 × 10⁻³ | 3.35 | SEYYSY |
| | | | | | DLFVY |
| h5E7-YD-F3 | hDLL3 | 3.12 × 105 | 4.05 × 10⁻⁴ | 1.29 | SSYWSY |
| | | | | | DLLVY |
| h5E7-YD-A6 | hDLL3 | 3.96 × 105 | 4.34 × 10⁻⁴ | 1.10 | SSYWSY |
| | | | | | DLFVY |
| h5E7-YD-B5 | hDLL3 | 4.74 × 105 | 1.30 × 10⁻⁴ | 0.27 | STYWSY |
| | | | | | DLFVY |

To assess the binding of the mAbs to DLL3 positive cells, human myeloma cell line RPMI-8226 was transduced to express the full-length extracellular portion of DLL3. Anti-DLL3 mAbs were diluted and incubated with DLL3⁺ RPMI-8226 cells. The cells were analyzed by flow cytometry and binding of a mAb was detected using a fluorophore conjugated anti-human IgG secondary antibody.

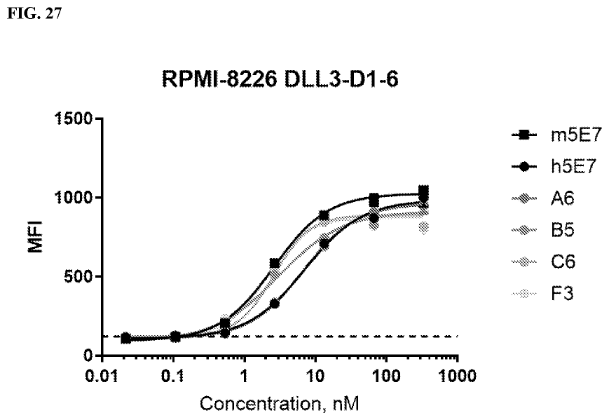

As shown in FIG. 27, conversion to a human backbone reduced the binding affinity of 5E7 to DLL3 presented on cells. After introducing mutations in heavy chain CDR3, the C6 and F3 variants of h5E7 showed similar binding affinities to cell surface DLL3 as the murine 5E7. The A6 variant exhibited similar binding affinity as h5E7, and the B5 variant bound with an intermediate affinity.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

1. An antibody heavy chain variable domain comprising an amino acid sequence at least 90% identical to the amino acid sequence of SEQ ID NO:33.

2. The antibody heavy chain variable domain according to claim 1, wherein the amino acid sequence comprises:
a. a complementarity-determining region 1 (CDR1) sequence represented by the amino acid sequence of SEQ ID NO:40; a complementarity-determining region 2 (CDR2) sequence represented by the amino acid sequence of SEQ ID NO:41; and a complementarity-determining region 3 (CDR3) sequence represented by the amino acid sequence of SEQ ID NO:50;
b. a CDR1 sequence represented by the amino acid sequence of SEQ ID NO:40; a CDR2 sequence represented by the amino acid sequence of SEQ ID NO:41; and a CDR3 sequence represented by the amino acid sequence of SEQ ID NO:47;
c. a CDR1 sequence represented by the amino acid sequence of SEQ ID NO:40; a CDR2 sequence represented by the amino acid sequence of SEQ ID NO:41; and a CDR3 sequence represented by the amino acid sequence of SEQ ID NO:46;
d. a CDR1 sequence represented by the amino acid sequence of SEQ ID NO:40; a CDR2 sequence represented by the amino acid sequence of SEQ ID NO:41; and a CDR3 sequence represented by the amino acid sequence of SEQ ID NO:49;
e. a CDR1 sequence represented by the amino acid sequence of SEQ ID NO:40; a CDR2 sequence represented by the amino acid sequence of SEQ ID NO:41; and a CDR3 sequence represented by the amino acid sequence of SEQ ID NO:48; or
f. a CDR1 sequence represented by the amino acid sequence of SEQ ID NO:40; a CDR2 sequence represented by the amino acid sequence of SEQ ID NO:41; and a CDR3 sequence represented by the amino acid sequence of SEQ ID NO:42.

3-7. (canceled)

8. An antibody heavy chain variable domain comprising an amino acid sequence at least 90% identical to an amino acid sequence selected from SEQ ID NO:1, SEQ ID NO:3, SEQ ID NO:5, and SEQ ID NO:7.

9. The antibody heavy chain variable domain according to claim 8, wherein the amino acid sequence comprises:
a. a CDR1 sequence represented by the amino acid sequence of SEQ ID NO:9; a CDR2 sequence represented by the amino acid sequence of SEQ ID NO:10; and a CDR3 sequence represented by the amino acid sequence of SEQ ID NO:11;
b. a CDR1 sequence represented by the amino acid sequence of SEQ ID NO:15; a CDR2 sequence represented by the amino acid sequence of SEQ ID NO:16; and a CDR3 sequence represented by the amino acid sequence of SEQ ID NO:17;
c. a CDR1 sequence represented by the amino acid sequence of SEQ ID NO:21; a CDR2 sequence represented by the amino acid sequence of SEQ ID NO:22; and a CDR3 sequence represented by the amino acid sequence of SEQ ID NO:23; or
d. a CDR1 sequence represented by the amino acid sequence of SEQ ID NO:27; a CDR2 sequence represented by the amino acid sequence of SEQ ID NO:28; and a CDR3 sequence represented by the amino acid sequence of SEQ ID NO:29.

10-21. (canceled)

22. An antigen-binding site comprising:
a. an antibody heavy chain variable domain comprising an amino acid sequence at least 90% identical to SEQ ID NO:33; wherein the amino acid sequence comprises:
i. a CDR1 sequence represented by the amino acid sequence of SEQ ID NO:40; a CDR2 sequence represented by the amino acid sequence of SEQ ID NO:41; and a CDR3 sequence represented by the amino acid sequence of SEO ID NO:50;
ii. a CDR1 sequence represented by the amino acid sequence of SEQ ID NO:40; a CDR2 sequence represented by the amino acid sequence of SEQ ID NO:41; and a CDR3 sequence represented by the amino acid sequence of SEQ ID NO:47;
iii. a CDR1 sequence represented by the amino acid sequence of SEQ ID NO:40; a CDR2 sequence represented by the amino acid sequence of SEQ ID NO:41; and a CDR3 sequence represented by the amino acid sequence of SEQ ID NO:46;
iv. a CDR1 sequence represented by the amino acid sequence of SEQ ID NO:40; a CDR2 sequence represented by the amino acid sequence of SEQ ID NO:41; and a CDR3 sequence represented by the amino acid sequence of SEQ ID NO:49;
v. a CDR1 sequence represented by the amino acid sequence of SEQ ID NO:40; a CDR2 sequence represented by the amino acid sequence of SEQ ID NO:41; and a CDR3 sequence represented by the amino acid sequence of SEQ ID NO:48; or
vi. a CDR1 sequence represented by the amino acid sequence of SEQ ID NO:40; a CDR2 sequence represented by the amino acid sequence of SEQ ID NO:41; and a CDR3 sequence represented by the amino acid sequence of SEQ ID NO:42; and
an antibody light chain variable domain comprising an amino acid sequence at least 90% identical to SEQ ID NO:34; and a CDR1 sequence represented by the amino acid sequence of SEQ ID NO:43, a CDR2 sequence represented by the amino acid sequence of SEQ ID NO:44, and a CDR3 sequence represented by the amino acid sequence of SEQ ID NO:45;
b. an antibody heavy chain variable domain comprising an amino acid sequence at least 90% identical to SEQ ID NO:1; wherein the amino acid sequence comprises a CDR1 sequence represented by the amino acid sequence of SEQ ID NO:9; a CDR2 sequence represented by the amino acid sequence of SEQ ID NO:10; and a CDR3 sequence represented by the amino acid sequence of SEQ ID NO:11; and an antibody light chain variable domain comprising an amino acid sequence at least 90% identical to SEQ ID NO:2; and a CDR1 sequence represented by the amino acid sequence of SEQ ID NO:12, a CDR2 sequence represented by the amino acid sequence of SEQ ID NO:13, and a CDR3 sequence represented by the amino acid sequence of SEQ ID NO:14;
c. an antibody heavy chain variable domain comprising an amino acid sequence at least 90% identical to SEQ ID NO:3; wherein the amino acid sequence comprises a CDR1 sequence represented by the amino acid sequence of SEQ ID NO:15; a CDR2 sequence represented by the amino acid sequence of SEQ ID NO:16; and a CDR3 sequence represented by the amino acid sequence of SEQ ID NO:17; and an antibody light chain variable domain comprising an amino acid sequence at least 90% identical to SEQ ID NO:4; and a CDR1 sequence represented by the amino acid sequence of SEQ ID NO:18, a CDR2 sequence represented by the amino acid sequence of SEQ ID NO:19, and a CDR3 sequence represented by the amino acid sequence of SEQ ID NO:20;
d. an antibody heavy chain variable domain comprising an amino acid sequence at least 90% identical to SEQ ID NO:5; wherein the amino acid sequence comprises a CDR1 sequence represented by the amino acid sequence of SEQ ID NO:21; a CDR2 sequence comprising an amino acid sequence identical to the amino acid sequence of sequence represented by the amino acid sequence of SEQ ID NO:22; and a CDR3 sequence represented by the amino acid sequence of SEQ ID NO:23; and an antibody light chain variable domain comprising an amino acid sequence at least 90% identical to SEQ ID NO:6; and a CDR1 sequence represented by the amino acid sequence of SEQ ID NO:24, a CDR2 sequence represented by the amino acid sequence of SEQ ID NO:25, and a CDR3 sequence represented by the amino acid sequence of SEQ ID NO:26; or
e. an antibody heavy chain variable domain comprising an amino acid sequence at least 90% identical to SEQ ID NO:7; wherein the amino acid sequence comprises a CDR1 sequence represented by the amino acid sequence of SEQ ID NO:27; a CDR2 sequence represented by the amino acid sequence of SEQ ID NO:28; and a CDR3 sequence represented by the amino acid sequence of SEQ ID NO:29; and an antibody light chain variable domain comprising an amino acid sequence at least 90% identical to SEQ ID NO:8; a CDR1 sequence represented by the amino acid sequence of SEQ ID NO:30, a CDR2 sequence represented by the amino acid sequence of SEQ ID NO:31, and a CDR3 sequence represented by the amino acid sequence of SEQ ID NO:32.

23-31. (canceled)

32. A protein comprising the antigen-binding site according to claim 22, wherein the antigen-binding site binds to human DLL3.

33. The protein of claim 32, wherein the protein further comprises a second antigen binding site that is the same or different from the antigen-binding site that binds to human DLL3.

34. The protein of claim 32, wherein the protein further comprises an antibody constant region that is at least 90% identical to human IgG1 constant region comprising two polypeptide chains, each of which comprises a hinge, CH2 and CH3 domain.

35-36. (canceled)

37. The protein of claim 34, wherein the amino acid sequence of one polypeptide chain of the antibody constant region differs from the amino acid sequence of an IgG1 constant region at one or more positions selected from Q347, Y349, L351, S354, E356, E357, K360, Q362, S364, T366, L368, K370, K392, T394, D399, S400, D401, F405, Y407, K409, T411, and K439; and the amino acid sequence of the other polypeptide chain of the antibody constant region differs from the amino acid sequence of an IgG1 constant region at one or more positions selected from Q347, Y349, L351, S354, E356, E357, S364, T366, L368, K370, N390, K392, T394, D399, D401, F405, Y407, K409, T411, and K439.

38-49. (canceled)

50. The protein of claim 34, wherein the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by Q347R, D399V, and F405T substitutions.

51-55. (canceled)

56. The protein of claim 50, wherein the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

57. (canceled)

58. A protein comprising an antigen-binding site that competes for binding to human DLL3 with:
a. an antibody comprising an antibody heavy chain having the amino acid sequence of SEQ ID NO:33 and an antibody light chain having the amino acid sequence of SEQ ID NO:34;
b. an antibody comprising an antibody heavy chain having the amino acid sequence of SEQ ID NO:1 and an antibody light chain having the amino acid sequence of SEQ ID NO:2;
c. an antibody comprising an antibody heavy chain having the amino acid sequence of SEQ ID NO:3 and an antibody light chain having the amino acid sequence of SEQ ID NO:4;
d. an antibody comprising an antibody heavy chain having the amino acid sequence of SEQ ID NO:5 and an antibody light chain having the amino acid sequence of SEQ ID NO:6; and/or
e. an antibody comprising an antibody heavy chain having the amino acid sequence of SEQ ID NO:7 and an antibody light chain having the amino acid sequence of SEQ ID NO:8.

59-62. (canceled)

63. A cell comprising one or more nucleic acids encoding the protein according to claim 32.

64. A formulation comprising the protein according to claim 32 and a pharmaceutically acceptable carrier.

65. A method of enhancing cancer cell death, the method comprising exposing a DLL3-expressing tumor to the protein according to claim 32.

66. A method of treating a DLL3-expressing cancer, the method comprising administering to a subject in need thereof the protein according to claim 32.

67. The method of claim 65, wherein the cancer is selected from the group consisting of small cell lung cancer, large cell neuroendocrine carcinoma, glioblastoma, Ewing's sarcoma, and cancers with neuroendocrine phenotype.

68. An isolated nucleic acid encoding a chimeric antigen receptor (CAR), wherein the nucleic acid comprises a nucleic acid sequence that encodes a DLL3-binding scFv of claim 105; a nucleic acid sequence encoding a transmembrane domain; and a nucleic acid sequence encoding an intracellular signaling domain.

69-75. (canceled)

76. An expression vector comprising the isolated nucleic acid of claim 68.

77. A chimeric antigen receptor (CAR), wherein the CAR comprises a DLL3-binding scFv of claim 105; a transmembrane domain; and an intracellular signaling domain.

78-84. (canceled)

85. An immune effector cell expressing the CAR of claim 77.

86-88. (canceled)

89. A DLL3/CD3-directed bispecific T-cell engager comprising a DLL3-binding scFv of claim 105.

90-92. (canceled)

93. An antibody-drug conjugate comprising a protein comprising a DLL3-binding scFv of claim 105.

94-96. (canceled)

97. An immunocytokine comprising a DLL3-binding scFv of claim 105, connected to a cytokine.

98-104. (canceled)

105. A DLL3-binding scFv comprising an amino acid sequence at least 90%, 95%, or 99% identical to an amino acid sequence selected from SEQ ID NO:51, SEQ ID NO:52, SEQ ID NO:55, SEQ ID NO:56, SEQ ID NO:59, SEQ ID NO:60, SEQ ID NO:63, SEQ ID NO:64, SEQ ID NO:67, and SEQ ID NO:68.


SEQUENCE LISTING (70 sequences)

SEQ ID NO: 1 - protein, 118 aa, Artificial Sequence
  Description of Artificial Sequence Synthetic polypeptide
QVQLQQSGAELMKPGASVKLSCKATGYTFTGYWIDWIKQRPGHGLEWVGEILPGSDNINYNEKFRGKATFTADTSSNTAYIQLSSLTTEDSAIYFCARCGTGPWFTYWGQGTLVTVSA

SEQ ID NO: 2 - protein, 113 aa, Artificial Sequence
  Description of Artificial Sequence Synthetic polypeptide
NIMMTQSPSSLAVSAGEKVTMSCKSSQSVLYSSNQKNYLAWYQQKPGQSPRLLIYWASTRASGVPDRFTGSGSGTDFTLTITNIQPEDLAVYYCHQFLSSTWTFGGGTKLEIK

SEQ ID NO: 3 - protein, 117 aa, Artificial Sequence
  Description of Artificial Sequence Synthetic polypeptide
QLQLVQSGPELMRPGETVKISCKASGYTFTTYGMNWVKQAPGKGLKWVGWINTYSGVPTYADDFKGRFAFSLESSASTAFLQINNLKDEDTATYFCARFGNYGFDCWGQGTTLTVSS

SEQ ID NO: 4 - protein, 113 aa, Artificial Sequence
  Description of Artificial Sequence Synthetic polypeptide
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHIYPYTFGGGTKLEIK

SEQ ID NO: 5 - protein, 120 aa, Artificial Sequence
  Description of Artificial Sequence Synthetic polypeptide
EVQLQQSGAELVRPGASVKLSCTASGFNIKDDYIHWVKQWPEQGLEWIGWIDSENGDTEYASKFQGKATMTADTSSNTAYLQLSGLTSEDTAVYYCTTSSYYSYDLFVYWGQGTLVTVSA

SEQ ID NO: 6 - protein, 111 aa, Artificial Sequence
  Description of Artificial Sequence Synthetic polypeptide
DVLMTQTPLTLSVPIGQPASISCKSSQSLLHSNGKTYLNWLLQRPGQSPKLLIYLVSKLESGVPDRFSGSGSGTDFTLKISRVEAEDLGVYYCLQTTHLYTFGGGTKLEIK

SEQ ID NO: 7 - protein, 117 aa, Artificial Sequence
  Description of Artificial Sequence Synthetic polypeptide
QIQLVQSGPELKKPGETVKISCKASGYTFTTYGVNWVKQAPGKGLKWMGWINTYSGVPTYADDFKGRFAFSLETIATTAYLQINNLKNEDTATYFCARFGNYGFDYWGQGTTLTVSS

SEQ ID NO: 8 - protein, 113 aa, Artificial Sequence
  Description of Artificial Sequence Synthetic polypeptide
DIVMTQSPSSLSVSAGEKVTMSCKSSQSLVNSGNQKNYLAWYQQKPGQPPKLLISGASTRESGVPDRFTGSGSGTDFTLTISSVQAEDLAVYYCQNDHNYPYTFGGGTKLEIK

SEQ ID NO: 9 - protein, 5 aa, Artificial Sequence
  Description of Artificial Sequence Synthetic peptide
GYWID

SEQ ID NO: 10 - protein, 17 aa, Artificial Sequence
  Description of Artificial Sequence Synthetic peptide
EILPGSDNINYNEKFRG

SEQ ID NO: 11 - protein, 9 aa, Artificial Sequence
  Description of Artificial Sequence Synthetic peptide
CGTGPWFTY

SEQ ID NO: 12 - protein, 17 aa, Artificial Sequence
  Description of Artificial Sequence Synthetic peptide
KSSQSVLYSSNQKNYLA

SEQ ID NO: 13 - protein, 7 aa, Artificial Sequence
  Description of Artificial Sequence Synthetic peptide
WASTRAS

SEQ ID NO: 14 - protein, 9 aa, Artificial Sequence
  Description of Artificial Sequence Synthetic peptide
HQFLSSTWT

SEQ ID NO: 15 - protein, 5 aa, Artificial Sequence
  Description of Artificial Sequence Synthetic peptide
TYGMN

SEQ ID NO: 16 - protein, 17 aa, Artificial Sequence
  Description of Artificial Sequence Synthetic peptide
WINTYSGVPTYADDFKG

SEQ ID NO: 17 - protein, 8 aa, Artificial Sequence
  Description of Artificial Sequence Synthetic peptide
FGNYGFDC

SEQ ID NO: 18 - protein, 17 aa, Artificial Sequence
  Description of Artificial Sequence Synthetic peptide
KSSQSLLNSGNQKNYLA

SEQ ID NO: 19 - protein, 7 aa, Artificial Sequence
  Description of Artificial Sequence Synthetic peptide
GASTRES

SEQ ID NO: 20 - protein, 9 aa, Artificial Sequence
  Description of Artificial Sequence Synthetic peptide
QNDHIYPYT

SEQ ID NO: 21 - protein, 5 aa, Artificial Sequence
  Description of Artificial Sequence Synthetic peptide
DDYIH

SEQ ID NO: 22 - protein, 17 aa, Artificial Sequence
  Description of Artificial Sequence Synthetic peptide
WIDSENGDTEYASKFQG

SEQ ID NO: 23 - protein, 11 aa, Artificial Sequence
  Description of Artificial Sequence Synthetic peptide
SSYYSYDLFVY

SEQ ID NO: 24 - protein, 16 aa, Artificial Sequence
  Description of Artificial Sequence Synthetic peptide
KSSQSLLHSNGKTYLN

SEQ ID NO: 25 - protein, 7 aa, Artificial Sequence
  Description of Artificial Sequence Synthetic peptide
LVSKLES

SEQ ID NO: 26 - protein, 8 aa, Artificial Sequence
  Description of Artificial Sequence Synthetic peptide
LQTTHLYT

SEQ ID NO: 27 - protein, 5 aa, Artificial Sequence
  Description of Artificial Sequence Synthetic peptide
TYGVN

SEQ ID NO: 28 - protein, 17 aa, Artificial Sequence
  Description of Artificial Sequence Synthetic peptide
WINTYSGVPTYADDFKG

SEQ ID NO: 29 - protein, 8 aa, Artificial Sequence
  Description of Artificial Sequence Synthetic peptide
FGNYGFDY

SEQ ID NO: 30 - protein, 17 aa, Artificial Sequence
  Description of Artificial Sequence Synthetic peptide
KSSQSLVNSGNQKNYLA

SEQ ID NO: 31 - protein, 7 aa, Artificial Sequence
  Description of Artificial Sequence Synthetic peptide
GASTRES

SEQ ID NO: 32 - protein, 9 aa, Artificial Sequence
  Description of Artificial Sequence Synthetic peptide
QNDHNYPYT

SEQ ID NO: 33 - protein, 120 aa, Artificial Sequence
  Description of Artificial Sequence Synthetic polypeptide
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSSYYSYDLFVYWGQGTLVTVSS

SEQ ID NO: 34 - protein, 111 aa, Artificial Sequence
  Description of Artificial Sequence Synthetic polypeptide
DVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGQGTKLEIK

SEQ ID NO: 35 - protein, 120 aa, Artificial Sequence
  Description of Artificial Sequence Synthetic polypeptide
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSEYYSYDLFVYWGQGTLVTVSS

SEQ ID NO: 36 - protein, 120 aa, Artificial Sequence
  Description of Artificial Sequence Synthetic polypeptide
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSSYWSYDLLVYWGQGTLVTVSS

SEQ ID NO: 37 - protein, 120 aa, Artificial Sequence
  Description of Artificial Sequence Synthetic polypeptide
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSSYWSYDLFVYWGQGTLVTVSS

SEQ ID NO: 38 - protein, 120 aa, Artificial Sequence
  Description of Artificial Sequence Synthetic polypeptide
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSTYWSYDLFVYWGQGTLVTVSS

SEQ ID NO: 39 - protein, 120 aa, Artificial Sequence
  Description of Artificial Sequence Synthetic polypeptide
  VARIANT (100)..(100): Ser, Thr or Glu
  VARIANT (102)..(102): Tyr or Trp
  VARIANT (107)..(107): Phe or Leu
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSXYXSYDLXVYWGQGTLVTVSS

SEQ ID NO: 40 - protein, 5 aa, Artificial Sequence
  Description of Artificial Sequence Synthetic peptide
DDYIH

SEQ ID NO: 41 - protein, 17 aa, Artificial Sequence
  Description of Artificial Sequence Synthetic peptide
WIDSENGDTEYASKFQG

SEQ ID NO: 42 - protein, 11 aa, Artificial Sequence
  Description of Artificial Sequence Synthetic peptide
SSYYSYDLFVY

SEQ ID NO: 43 - protein, 16 aa, Artificial Sequence
  Description of Artificial Sequence Synthetic peptide
KSSQSLLHSNGKTYLN

SEQ ID NO: 44 - protein, 7 aa, Artificial Sequence
  Description of Artificial Sequence Synthetic peptide
LVSKLES

SEQ ID NO: 45 - protein, 8 aa, Artificial Sequence
  Description of Artificial Sequence Synthetic peptide
LQTTHLYT

SEQ ID NO: 46 - protein, 11 aa, Artificial Sequence
  Description of Artificial Sequence Synthetic peptide
SEYYSYDLFVY

SEQ ID NO: 47 - protein, 11 aa, Artificial Sequence
  Description of Artificial Sequence Synthetic peptide
SSYWSYDLLVY

SEQ ID NO: 48 - protein, 11 aa, Artificial Sequence
  Description of Artificial Sequence Synthetic peptide
SSYWSYDLFVY

SEQ ID NO: 49 - protein, 11 aa, Artificial Sequence
  Description of Artificial Sequence Synthetic peptide
STYWSYDLFVY

SEQ ID NO: 50 - protein, 11 aa, Artificial Sequence
  Description of Artificial Sequence Synthetic peptide
  MOD_RES (2)..(2): Ser, Thr or Glu
  MOD_RES (4)..(4): Tyr or Trp
  MOD_RES (9)..(9): Phe or Leu
SXYXSYDLXVY

SEQ ID NO: 51 - protein, 251 aa, Artificial Sequence
  Description of Artificial Sequence Synthetic polypeptide
QVQLVQSGAEVKKPGASVKVSCKASGFNIKDDYIHWVRQAPGQCLEWMGWIDSENGDTEYASKFQGRVTITADTSANTAYMELSSLRSEDTAVYYCATSSYYSYDLFVYWGQGTLVTVSSGGGGSGGGGSGGGGSGGGGSDVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGCGTKLEIK

SEQ ID NO: 52 - protein, 251 aa, Artificial Sequence
  Description of Artificial Sequence Synthetic polypeptide
DVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGCGTKLEIKGGGGSGGGGSGGGGSGGGGSQVQLVQSGAEVKKPGASVKVSCKASGFNIKDDYIHWVRQAPGQCLEWMGWIDSENGDTEYASKFQGRVTITADTSANTAYMELSSLRSEDTAVYYCATSSYYSYDLFVYWGQGTLVTVSS

SEQ ID NO: 53 - DNA, 753 bp, Artificial Sequence
  Description of Artificial Sequence Nucleotide sequence encoding synthetic polypeptide
caggtgcagctggtgcagagcggcgccgaggtgaagaagcccggcgccagcgtgaaggtgagctgcaaggccagcggcttcaacatcaaggacgactacatccactgggtgaggcaggcccccggccagtgcctggagtggatgggctggatcgacagcgagaacggcgacaccgagtacgccagcaagttccagggcagggtgaccatcaccgccgacaccagcgccaacaccgcctacatggagctgagcagcctgaggagcgaggacaccgccgtgtactactgcgccaccagcagctactacagctacgacctgttcgtgtactggggccagggcaccctggtgaccgtgagcagcggcggcggcggcagcggcggcggcggcagcggcggcggcggcagcggcggcggcggcagcgacgtgcagatgacccagagccccagcagcctgagcgccagcgtgggcgacagggtgaccatcacctgcaagagcagccagagcctgctgcacagcaacggcaagacctacctgaactggctgcagcagaagcccggccaggcccccaagctgctgctgtacctggtgagcaagctggagagcggcgtgcccagcaggttcagcggcagcggcagcggcaccgactacaccctgaccatcagcagcctgcagcccgaggacttcgccacctactactgcctgcagaccacccacctgtacaccttcggctgcggcaccaagctggagatcaag

SEQ ID NO: 54 - DNA, 753 bp, Artificial Sequence
  Description of Artificial Sequence Nucleotide sequence encoding synthetic polypeptide
gacgtgcagatgacccagagccccagcagcctgagcgccagcgtgggcgacagggtgaccatcacctgcaagagcagccagagcctgctgcacagcaacggcaagacctacctgaactggctgcagcagaagcccggccaggcccccaagctgctgctgtacctggtgagcaagctggagagcggcgtgcccagcaggttcagcggcagcggcagcggcaccgactacaccctgaccatcagcagcctgcagcccgaggacttcgccacctactactgcctgcagaccacccacctgtacaccttcggctgcggcaccaagctggagatcaagggcggcggcggcagcggcggcggcggcagcggcggcggcggcagcggcggcggcggcagccaggtgcagctggtgcagagcggcgccgaggtgaagaagcccggcgccagcgtgaaggtgagctgcaaggccagcggcttcaacatcaaggacgactacatccactgggtgaggcaggcccccggccagtgcctggagtggatgggctggatcgacagcgagaacggcgacaccgagtacgccagcaagttccagggcagggtgaccatcaccgccgacaccagcgccaacaccgcctacatggagctgagcagcctgaggagcgaggacaccgccgtgtactactgcgccaccagcagctactacagctacgacctgttcgtgtactggggccagggcaccctggtgaccgtgagcagc

SEQ ID NO: 55 - protein, 251 aa, Artificial Sequence
  Description of Artificial Sequence Synthetic polypeptide
QVQLVQSGAEVKKPGASVKVSCKASGFNIKDDYIHWVRQAPGQCLEWMGWIDSENGDTEYASKFQGRVTITADTSANTAYMELSSLRSEDTAVYYCATSEYYSYDLFVYWGQGTLVTVSSGGGGSGGGGSGGGGSGGGGSDVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGCGTKLEIK

SEQ ID NO: 56 - protein, 251 aa, Artificial Sequence
  Description of Artificial Sequence Synthetic polypeptide
DVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGCGTKLEIKGGGGSGGGGSGGGGSGGGGSQVQLVQSGAEVKKPGASVKVSCKASGFNIKDDYIHWVRQAPGQCLEWMGWIDSENGDTEYASKFQGRVTITADTSANTAYMELSSLRSEDTAVYYCATSEYYSYDLFVYWGQGTLVTVSS

SEQ ID NO: 57 - DNA, 753 bp, Artificial Sequence
  Description of Artificial Sequence Nucleotide sequence encoding synthetic polypeptide
caggtgcagctggtgcagagcggcgccgaggtgaagaagcccggcgccagcgtgaaggtgagctgcaaggccagcggcttcaacatcaaggacgactacatccactgggtgaggcaggcccccggccagtgcctggagtggatgggctggatcgacagcgagaacggcgacaccgagtacgccagcaagttccagggcagggtgaccatcaccgccgacaccagcgccaacaccgcctacatggagctgagcagcctgaggagcgaggacaccgccgtgtactactgcgccaccagcgagtactacagctacgacctgttcgtgtactggggccagggcaccctggtgaccgtgagcagcggcggcggcggcagcggcggcggcggcagcggcggcggcggcagcggcggcggcggcagcgacgtgcagatgacccagagccccagcagcctgagcgccagcgtgggcgacagggtgaccatcacctgcaagagcagccagagcctgctgcacagcaacggcaagacctacctgaactggctgcagcagaagcccggccaggcccccaagctgctgctgtacctggtgagcaagctggagagcggcgtgcccagcaggttcagcggcagcggcagcggcaccgactacaccctgaccatcagcagcctgcagcccgaggacttcgccacctactactgcctgcagaccacccacctgtacaccttcggctgcggcaccaagctggagatcaag

SEQ ID NO: 58 - DNA, 753 bp, Artificial Sequence
  Description of Artificial Sequence Nucleotide sequence encoding synthetic polypeptide
gacgtgcagatgacccagagccccagcagcctgagcgccagcgtgggcgacagggtgaccatcacctgcaagagcagccagagcctgctgcacagcaacggcaagacctacctgaactggctgcagcagaagcccggccaggcccccaagctgctgctgtacctggtgagcaagctggagagcggcgtgcccagcaggttcagcggcagcggcagcggcaccgactacaccctgaccatcagcagcctgcagcccgaggacttcgccacctactactgcctgcagaccacccacctgtacaccttcggctgcggcaccaagctggagatcaagggcggcggcggcagcggcggcggcggcagcggcggcggcggcagcggcggcggcggcagccaggtgcagctggtgcagagcggcgccgaggtgaagaagcccggcgccagcgtgaaggtgagctgcaaggccagcggcttcaacatcaaggacgactacatccactgggtgaggcaggcccccggccagtgcctggagtggatgggctggatcgacagcgagaacggcgacaccgagtacgccagcaagttccagggcagggtgaccatcaccgccgacaccagcgccaacaccgcctacatggagctgagcagcctgaggagcgaggacaccgccgtgtactactgcgccaccagcgagtactacagctacgacctgttcgtgtactggggccagggcaccctggtgaccgtgagcagc

SEQ ID NO: 59 - protein, 251 aa, Artificial Sequence
  Description of Artificial Sequence Synthetic polypeptide
QVQLVQSGAEVKKPGASVKVSCKASGFNIKDDYIHWVRQAPGQCLEWMGWIDSENGDTEYASKFQGRVTITADTSANTAYMELSSLRSEDTAVYYCATSSYWSYDLLVYWGQGTLVTVSSGGGGSGGGGSGGGGSGGGGSDVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGCGTKLEIK

SEQ ID NO: 60 - protein, 251 aa, Artificial Sequence
  Description of Artificial Sequence Synthetic polypeptide
DVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGCGTKLEIKGGGGSGGGGSGGGGSGGGGSQVQLVQSGAEVKKPGASVKVSCKASGFNIKDDYIHWVRQAPGQCLEWMGWIDSENGDTEYASKFQGRVTITADTSANTAYMELSSLRSEDTAVYYCATSSYWSYDLLVYWGQGTLVTVSS

SEQ ID NO: 61 - DNA, 753 bp, Artificial Sequence
  Description of Artificial Sequence Nucleotide sequence encoding synthetic polypeptide
caggtgcagctggtgcagagcggcgccgaggtgaagaagcccggcgccagcgtgaaggtgagctgcaaggccagcggcttcaacatcaaggacgactacatccactgggtgaggcaggcccccggccagtgcctggagtggatgggctggatcgacagcgagaacggcgacaccgagtacgccagcaagttccagggcagggtgaccatcaccgccgacaccagcgccaacaccgcctacatggagctgagcagcctgaggagcgaggacaccgccgtgtactactgcgccaccagcagctactggagctacgacctgctggtgtactggggccagggcaccctggtgaccgtgagcagcggcggcggcggcagcggcggcggcggcagcggcggcggcggcagcggcggcggcggcagcgacgtgcagatgacccagagccccagcagcctgagcgccagcgtgggcgacagggtgaccatcacctgcaagagcagccagagcctgctgcacagcaacggcaagacctacctgaactggctgcagcagaagcccggccaggcccccaagctgctgctgtacctggtgagcaagctggagagcggcgtgcccagcaggttcagcggcagcggcagcggcaccgactacaccctgaccatcagcagcctgcagcccgaggacttcgccacctactactgcctgcagaccacccacctgtacaccttcggctgcggcaccaagctggagatcaag

SEQ ID NO: 62 - DNA, 753 bp, Artificial Sequence
  Description of Artificial Sequence Nucleotide sequence encoding synthetic polypeptide
gacgtgcagatgacccagagccccagcagcctgagcgccagcgtgggcgacagggtgaccatcacctgcaagagcagccagagcctgctgcacagcaacggcaagacctacctgaactggctgcagcagaagcccggccaggcccccaagctgctgctgtacctggtgagcaagctggagagcggcgtgcccagcaggttcagcggcagcggcagcggcaccgactacaccctgaccatcagcagcctgcagcccgaggacttcgccacctactactgcctgcagaccacccacctgtacaccttcggctgcggcaccaagctggagatcaagggcggcggcggcagcggcggcggcggcagcggcggcggcggcagcggcggcggcggcagccaggtgcagctggtgcagagcggcgccgaggtgaagaagcccggcgccagcgtgaaggtgagctgcaaggccagcggcttcaacatcaaggacgactacatccactgggtgaggcaggcccccggccagtgcctggagtggatgggctggatcgacagcgagaacggcgacaccgagtacgccagcaagttccagggcagggtgaccatcaccgccgacaccagcgccaacaccgcctacatggagctgagcagcctgaggagcgaggacaccgccgtgtactactgcgccaccagcagctactggagctacgacctgctggtgtactggggccagggcaccctggtgaccgtgagcagc

SEQ ID NO: 63 - protein, 251 aa, Artificial Sequence
  Description of Artificial Sequence Synthetic polypeptide
QVQLVQSGAEVKKPGASVKVSCKASGFNIKDDYIHWVRQAPGQCLEWMGWIDSENGDTEYASKFQGRVTITADTSANTAYMELSSLRSEDTAVYYCATSSYWSYDLFVYWGQGTLVTVSSGGGGSGGGGSGGGGSGGGGSDVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGCGTKLEIK

SEQ ID NO: 64 - protein, 251 aa, Artificial Sequence
  Description of Artificial Sequence Synthetic polypeptide
DVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGCGTKLEIKGGGGSGGGGSGGGGSGGGGSQVQLVQSGAEVKKPGASVKVSCKASGFNIKDDYIHWVRQAPGQCLEWMGWIDSENGDTEYASKFQGRVTITADTSANTAYMELSSLRSEDTAVYYCATSSYWSYDLFVYWGQGTLVTVSS

SEQ ID NO: 65 - DNA, 753 bp, Artificial Sequence
  Description of Artificial Sequence Nucleotide sequence encoding synthetic polypeptide
caggtgcagctggtgcagagcggcgccgaggtgaagaagcccggcgccagcgtgaaggtgagctgcaaggccagcggcttcaacatcaaggacgactacatccactgggtgaggcaggcccccggccagtgcctggagtggatgggctggatcgacagcgagaacggcgacaccgagtacgccagcaagttccagggcagggtgaccatcaccgccgacaccagcgccaacaccgcctacatggagctgagcagcctgaggagcgaggacaccgccgtgtactactgcgccaccagcagctactggagctacgacctgttcgtgtactggggccagggcaccctggtgaccgtgagcagcggcggcggcggcagcggcggcggcggcagcggcggcggcggcagcggcggcggcggcagcgacgtgcagatgacccagagccccagcagcctgagcgccagcgtgggcgacagggtgaccatcacctgcaagagcagccagagcctgctgcacagcaacggcaagacctacctgaactggctgcagcagaagcccggccaggcccccaagctgctgctgtacctggtgagcaagctggagagcggcgtgcccagcaggttcagcggcagcggcagcggcaccgactacaccctgaccatcagcagcctgcagcccgaggacttcgccacctactactgcctgcagaccacccacctgtacaccttcggctgcggcaccaagctggagatcaag

SEQ ID NO: 66 - DNA, 753 bp, Artificial Sequence
  Description of Artificial Sequence Nucleotide sequence encoding synthetic polypeptide
gacgtgcagatgacccagagccccagcagcctgagcgccagcgtgggcgacagggtgaccatcacctgcaagagcagccagagcctgctgcacagcaacggcaagacctacctgaactggctgcagcagaagcccggccaggcccccaagctgctgctgtacctggtgagcaagctggagagcggcgtgcccagcaggttcagcggcagcggcagcggcaccgactacaccctgaccatcagcagcctgcagcccgaggacttcgccacctactactgcctgcagaccacccacctgtacaccttcggctgcggcaccaagctggagatcaagggcggcggcggcagcggcggcggcggcagcggcggcggcggcagcggcggcggcggcagccaggtgcagctggtgcagagcggcgccgaggtgaagaagcccggcgccagcgtgaaggtgagctgcaaggccagcggcttcaacatcaaggacgactacatccactgggtgaggcaggcccccggccagtgcctggagtggatgggctggatcgacagcgagaacggcgacaccgagtacgccagcaagttccagggcagggtgaccatcaccgccgacaccagcgccaacaccgcctacatggagctgagcagcctgaggagcgaggacaccgccgtgtactactgcgccaccagcagctactggagctacgacctgttcgtgtactggggccagggcaccctggtgaccgtgagcagc

SEQ ID NO: 67 - protein, 251 aa, Artificial Sequence
  Description of Artificial Sequence Synthetic polypeptide
QVQLVQSGAEVKKPGASVKVSCKASGFNIKDDYIHWVRQAPGQCLEWMGWIDSENGDTEYASKFQGRVTITADTSANTAYMELSSLRSEDTAVYYCATSTYWSYDLFVYWGQGTLVTVSSGGGGSGGGGSGGGGSGGGGSDVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGCGTKLEIK

SEQ ID NO: 68 - protein, 251 aa, Artificial Sequence
  Description of Artificial Sequence Synthetic polypeptide
DVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGCGTKLEIKGGGGSGGGGSGGGGSGGGGSQVQLVQSGAEVKKPGASVKVSCKASGFNIKDDYIHWVRQAPGQCLEWMGWIDSENGDTEYASKFQGRVTITADTSANTAYMELSSLRSEDTAVYYCATSTYWSYDLFVYWGQGTLVTVSS

SEQ ID NO: 69 - DNA, 753 bp, Artificial Sequence
  Description of Artificial Sequence Nucleotide sequence encoding synthetic polypeptide
caggtgcagctggtgcagagcggcgccgaggtgaagaagcccggcgccagcgtgaaggtgagctgcaaggccagcggcttcaacatcaaggacgactacatccactgggtgaggcaggcccccggccagtgcctggagtggatgggctggatcgacagcgagaacggcgacaccgagtacgccagcaagttccagggcagggtgaccatcaccgccgacaccagcgccaacaccgcctacatggagctgagcagcctgaggagcgaggacaccgccgtgtactactgcgccaccagcacctactggagctacgacctgttcgtgtactggggccagggcaccctggtgaccgtgagcagcggcggcggcggcagcggcggcggcggcagcggcggcggcggcagcggcggcggcggcagcgacgtgcagatgacccagagccccagcagcctgagcgccagcgtgggcgacagggtgaccatcacctgcaagagcagccagagcctgctgcacagcaacggcaagacctacctgaactggctgcagcagaagcccggccaggcccccaagctgctgctgtacctggtgagcaagctggagagcggcgtgcccagcaggttcagcggcagcggcagcggcaccgactacaccctgaccatcagcagcctgcagcccgaggacttcgccacctactactgcctgcagaccacccacctgtacaccttcggctgcggcaccaagctggagatcaag

SEQ ID NO: 70 - DNA, 753 bp, Artificial Sequence
  Description of Artificial Sequence Nucleotide sequence encoding synthetic polypeptide
gacgtgcagatgacccagagccccagcagcctgagcgccagcgtgggcgacagggtgaccatcacctgcaagagcagccagagcctgctgcacagcaacggcaagacctacctgaactggctgcagcagaagcccggccaggcccccaagctgctgctgtacctggtgagcaagctggagagcggcgtgcccagcaggttcagcggcagcggcagcggcaccgactacaccctgaccatcagcagcctgcagcccgaggacttcgccacctactactgcctgcagaccacccacctgtacaccttcggctgcggcaccaagctggagatcaagggcggcggcggcagcggcggcggcggcagcggcggcggcggcagcggcggcggcggcagccaggtgcagctggtgcagagcggcgccgaggtgaagaagcccggcgccagcgtgaaggtgagctgcaaggccagcggcttcaacatcaaggacgactacatccactgggtgaggcaggcccccggccagtgcctggagtggatgggctggatcgacagcgagaacggcgacaccgagtacgccagcaagttccagggcagggtgaccatcaccgccgacaccagcgccaacaccgcctacatggagctgagcagcctgaggagcgaggacaccgccgtgtactactgcgccaccagcacctactggagctacgacctgttcgtgtactggggccagggcaccctggtgaccgtgagcagc